(19) United States
(12) Patent Application Publication
(10) Pub. No.: US 2013/0039928 A1
(43) Pub. Date: Feb. 14, 2013

(54) COMPOSITIONS AND METHODS FOR REVERSING CORTICOSTEROID RESISTANCE OR TREATING RESPIRATORY INFECTIONS

(71) Applicants: Shyam Biswal, Ellicott City, MD (US); Rajesh K. Thimmulappa, Baltimore, MD (US)

(72) Inventors: Shyam Biswal, Ellicott City, MD (US); Rajesh K. Thimmulappa, Baltimore, MD (US)

(73) Assignee: THE JOHNS HOPKINS UNIVERSITY, Baltimore, MD (US)

(21) Appl. No.: 13/575,880

(22) PCT Filed: Jan. 28, 2011
(86) PCT No.: PCT/US11/23003

§ 371 Date: Oct. 22, 2012

Related U.S. Application Data
(60) Provisional application No. 61/298,968, filed on Jan. 28, 2010.

(51) Int. Cl. A61K 31/26; A61K 31/573; A61K 31/58; C12N 5/0786; A61K 39/395; A61P 11/06; A61P 11/00; A61P 37/06; A61P 27/16; A61P 1/00; A61P 29/00; A61P 19/02; A61P 31/00; A61P 31/04; A61P 31/16; A61P 31/20; A61P 31/14; A61K 31/56

(52) U.S. Cl. 424/172.1; 514/514; 514/171; 435/375; 514/515

(57) ABSTRACT

The present invention features methods for reversing corticosteroid resistance, as well as for the treatment of respiratory infections, particularly those associated with chronic obstructive pulmonary disease. In one embodiment, the method increases Nrf2 biological activity or expression.

### CROSS-REFERENCE TO RELATED APPLICATION

This application claims the benefit of the following U.S. Provisional Application No. 61/298,968, filed Jan. 28, 2010, the entire disclosure of which are hereby incorporated in its entirety.

### STATEMENT OF RIGHTS TO INVENTIONS MADE UNDER FEDERALLY SPONSORED RESEARCH

This work was supported by the following grants from the National Institutes of Health, Grant Nos: This work was supported by the following grants from the National Institutes of Health, Grant Nos: HL081205, P50HL084945, P50ES015903 and ES03819. The government may have certain rights in the invention.

### BACKGROUND OF THE INVENTION

Chronic obstructive pulmonary disease (COPD) is characterized by a progressive decrease in lung function and encompasses both chronic bronchitis and emphysema. COPD is the fifth leading cause of death worldwide and tobacco exposure is the major risk factor for COPD development in industrialized countries. Patients with COPD have frequent symptomatic exacerbations, which are primarily due to exposure to bacterial or viral infections or environmental pollutants. These exacerbations are a major cause of morbidity, mortality, and healthcare costs. Among the bacterial causes of exacerbations, nontypeable Haemophilus influenzae (NTHI) is the most prevalent, and Pseudomonas aeruginosa (PA) becomes important in severe COPD. Patients with advanced COPD experience, on average, two to three periods of exacerbation annually. Clinical and animal studies have shown that cigarette smoking causes defective bacterial phagocytosis by alveolar macrophages resulting in bacterial colonization and enhanced inflammation in lungs. Currently, there are no proven therapies that can inhibit bacterial colonization and prevent infectious COPD exacerbations.

Current treatments for COPD are of limited benefit. Corticosteroids are highly effective anti-inflammatory drugs for asthma, but they have little therapeutic benefit in COPD because of diminished corticosteroid sensitivity. High doses of inhaled corticosteroids are widely used widely to manage COPD; but they reduce exacerbations by only about 20% to 25% and do not alter disease progression or survival. High doses of systemic corticosteroids are used to treat acute severe COPD exacerbations, but they reduce length of hospitalization by only 9%. Therefore, treatments aimed at improving cortico steroid resistance in COPD may be of substantial benefit.

### SUMMARY OF THE INVENTION

As described below, the present invention features compositions and methods for reversing corticosteroid resistance and for treating or preventing bacterial infections, particularly those infections associated with chronic obstructive pulmonary disease.

In one aspect, the invention provides a method of restoring corticosteroid responsiveness in a subject in need thereof, the method involving contacting a cell of the subject with an agent (e.g., sulforaphane or another agent listed in Table 1) that increases Nrf2 biological activity in the cell, thereby restoring corticosteroid responsiveness in the subject. In one embodiment, the subject has chronic obstructive pulmonary disease (COPD), asthma, severe asthma, acute graft-versus host disease, autoimmune inner ear disease, inflammatory bowel disease, or rheumatoid arthritis.

In another aspect, the invention provides a method of restoring corticosteroid responsiveness in a subject with chronic obstructive pulmonary disease (COPD) containing administering an effective amount of an agent that stimulates Nrf2 activity to the subject, thereby restoring corticosteroid responsiveness in the subject.

In another aspect, the invention provides a method of treating or preventing a respiratory infection in a subject, the method involves contacting a cell (e.g., alveolar macrophage) of the subject with an agent that increases Nrf2 biological activity in the cell, thereby treating or preventing the respiratory infection. In one embodiment, the subject has an acute respiratory infection, chronic bronchitis, cystic fibrosis or an immunodeficiency syndrome that reduces or otherwise compromises the efficacy of the subject's immune system. In another embodiment, the subject is a smoker, has emphysema, or has COPD. In yet another embodiment, the infection is associated with Pseudomonas aeruginosa, nontypeable Haemophilus influenzae, Moraxella catarrhalis, streptococcus pneumonia, staphylococcus aureus, Rhinovirus, coronovirus, influenza A and B, parainfluenza, Adenovirus, or Respiratory syncytial virus.

In another aspect, the invention provides a method for increasing macrophage bactericidal activity, the method involves contacting a macrophage with an agent that increases Nrf2 activity, thereby increasing macrophage bactericidal activity.

In another aspect, the invention provides a method of increasing bacterial phagocytosis by a macrophage involving contacting the macrophage with an agent that increases Nrf2 activity, thereby increasing bacterial phagocytosis by the macrophage. In one embodiment, the macrophage is an alveolar macrophage.

In another aspect, the invention provides a method of treating or preventing bacterial colonization in a tissue or organ of a subject, the method involving contacting the tissue or organ of the subject with an agent that increases Nrf2 biological activity, thereby treating or preventing bacterial colonization and inflammation.

In another aspect, the invention provides a method for treating an infection in a subject having or at risk of developing chronic obstructive pulmonary disease (COPD), the method involving administering an effective amount of a Keap1 inhibitor to a subject in need thereof, thereby treating chronic obstructive pulmonary disease.

In another aspect, the invention provides a method for reversing corticosteroid resistance in a subject having COPD, the method involving administering an effective amount of an agent that increases Nrf2 biological activity and a corticosteroid to a subject in need thereof, thereby treating or preventing corticosteroid resistance in the subject. In one embodiment, the subject has a respiratory infection.

In another aspect, the invention provides a method for treating or preventing a pulmonary infection in a subject having or at risk of developing COPD, the method involving administering an effective amount of an agent that increases Nrf2 biological activity and a corticosteroid to a subject in need thereof, thereby treating or preventing a pulmonary infection in the subject. In one embodiment, the infection is associated with Pseudomonas aeruginosa, nontypeable Haemophilus influenzae, Moraxella catarrhalis, streptococcus pneumonia, staphylococcus aureus and Rhinovirus, coronovirus, influenza A and B, parainfluenza, Adenovirus, and Respiratory syncytial virus.

In another aspect, the invention provides a pharmaceutical composition for the treatment or prevention of a pulmonary inflammatory condition containing a therapeutically effective amount of an agent that increases a Nrf2 biological activity or Nrf2 expression and an effective amount of a corticosteroid in a pharmaceutically acceptable excipient.

In another aspect, the invention provides a pharmaceutical composition for the treatment or prevention of corticosteroid resistance involving a therapeutically effective amount of an agent that increases a Nrf2 biological activity or Nrf2 expression and an effective amount of a corticosteroid in a pharmaceutically acceptable excipient. In one embodiment, the compound is sulforaphane or another agent listed in Table 1. In another embodiment, the corticosteroid is selected from the group consisting of dexamethasone, flunisolide, fluticasone propionate, triamcinolone acetonide, beclomethasone dipropionate, budesonide, prednisone, prednisolone, and methylprednisolone. In another embodiment, the agent reduces Keap1 inhibition of Nrf2. In yet another embodiment, the agent is an inhibitory nucleic acid molecule (e.g., an siRNA, an antisense RNA, a ribozyme, or a shRNA) that decreases the expression of a Keap1 polypeptide or nucleic acid molecule. In another embodiment, the agent disrupts Keap1 binding to Nrf2. In another embodiment, the agent is an antibody or peptide. In yet another embodiment, the pharmaceutical composition is formulated for inhalation or oral administration.

In another aspect, the invention provides a pharmaceutical composition for treating or preventing a respiratory infection, the composition containing an effective amount of an agent that increases a Nrf2 biological activity or Nrf2 expression and an effective amount of an antibiotic.

In another aspect, the invention provides a device for dispersing an effective amount of an agent that increases a Nrf2 biological activity or Nrf2 expression and an effective amount of a corticosteroid into particles and delivering a dose of the particles to lung tissue of a subject. In one embodiment, the device is a nebulizer, metered dose inhaler or dry powder inhaler.

In another aspect, the invention provides a kit for reversing corticosteroid resistance, the kit containing an effective amount of an agent that increases a Nrf2 biological activity or Nrf2 expression and an effective amount of a corticosteroid and instructions for the use of the kit in a method of the invention.

In another aspect, the invention provides a kit for treating a respiratory infection, the kit containing an effective amount of an agent that increases a Nrf2 biological activity or Nrf2 expression and an effective amount of a corticosteroid in a pharmaceutically acceptable excipient, and instructions for the use of the kit in a method of the invention.

In various embodiments of the above aspects or of any other aspect of the invention delineated herein, the method reverses corticosteroid resistance or is useful for the treatment of a respiratory infection or other pulmonary condition. In particular embodiments, the agent is sulforaphane or another agent listed in Table 1. Such agents are administered alone or in combination with a corticosteroid (e.g., dexamethasone, flunisolide, fluticasone propionate, triamcinolone acetonide, beclomethasone dipropionate, budesonide, prednisone, prednisolone, and methylprednisolone). In other embodiments, sulforaphane or another agent listed in Table 1 is administered in combination with an antibiotic. In various embodiments, the method increases Nrf2 transcription or translation. In other embodiments, a composition of the invention is administered to an alveolar macrophage, a respiratory tissue (e.g., a mucous membrane) and/or a pulmonary organ (e.g., lung). In still other embodiments, the agent increases a Nrf2 biological activity selected from the group consisting of binding to an antioxidant-response element (ARE), nuclear accumulation, or the transcriptional induction of target genes. In one embodiment, the target gene is Marco. In other embodiments, the agent increases secretion of secretory leukocyte protease inhibitor. In other embodiments, the agent increases macrophage bacterial recognition, phagocytosis and/or clearance. In still other embodiments, the agent reduces Keap1 inhibition of Nrf2. In other embodiments, the agent is an inhibitory nucleic acid molecule that decreases the expression of a Keap1 polypeptide or nucleic acid molecule. In other embodiments, the inhibitory nucleic acid molecule is an siRNA, an antisense RNA, a ribozyme, or a shRNA. In other embodiments, the agent disrupts Keap1 binding to Nrf2. In another embodiment, the agent is an antibody or peptide. In another embodiment, the method restores corticosteroid responsiveness. In another embodiment, the method increases phagocytosis in alveolar macrophages. In another embodiment, the method increases scavenger receptor MARCO expression, increases the activity of histone deacetylase 2 (HDAC2), and or reduces Keap1 inhibition of Nrf2.

Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound.

By “ameliorate” is meant decrease, suppress, attenuate, diminish, arrest, or stabilize the development or progression of a disease.

By “corticosteroid resistance” is meant having diminished corticosteroid sensitivity. Conditions associated with corticosteroid resistance include, but are not limited to, corticosteroid resistance in COPD, asthma, including severe asthma, acute graft-versus host disease, autoimmune inner ear disease, inflammatory bowel diseases, rheumatoid arthritis, as well as bacterial infections, including those associated with COPD and related conditions (e.g. smoking, chronic bronchitis).

By “Nrf2 expression or biological activity” is meant binding to an antioxidant-response element (ARE), nuclear accumulation, the transcriptional induction of target genes, or binding to a Keap1 polypeptide.

By “Keap1 polypeptide” is meant a polypeptide comprising an amino acid sequence having at least 85% identity to GenBank Accession No. AAH21957. An exemplary Keap1 polypeptide sequence is provided below:

```
    1 nnspdgntds saldcynpmt nqwspcapms vprnrigvgv idghiyavgg shgcihhnsv
  61 eryeperdew hlvapmltrr igvgvavlnr llyavggfdg tnrlnsaecy ypernewrmi
121 tamntirsga gvcvlhnciy aaggydgqdq lnsverydve tetwtfvapm khrrsalgit
181 vhqgriyvlg gydghtflds vecydpdtdt wsevtrmtsg rsgvgvavtm epcrkqidqq
241 nctc
```

By “Keap1 nucleic acid molecule” is meant a nucleic acid molecule that encodes a Keap1 polypeptide or fragment thereof. An exemplary Keap1 nucleic acid sequence is provided below:

```
    1 gaacaactcg cccgacggca acaccgactc cagcgccctg gactgttaca accccatgac
  61 caatcagtgg tcgccctgcg cccccatgag cgtgccccgt aaccgcatcg gggtgggggt
121 catcgatggc cacatctatg ccgtcggcgg ctcccacggc tgcatccacc acaacagtgt
181 ggagaggtat gagccagagc gggatgagtg gcacttggtg gccccaatgc tgacacgaag
241 gatcggggtg ggcgtggctg tcctcaatcg tctcctttat gccgtggggg gctttgacgg
301 gacaaaccgc cttaattcag ctgagtgtta ctacccagag aggaacgagt ggcgaatgat
361 cacagcaatg aacaccatcc gaagcggggc aggcgtctgc gtcctgcaca actgtatcta
421 tgctgctggg ggctatgatg gtcaggacca gctgaacagc gtggagcgct acgatgtgga
481 aacagagacg tggactttcg tagcccccat gaagcaccgg cgaagtgccc tggggatcac
541 tgtccaccag gggagaatct acgtccttgg aggctatgat ggtcacacgt tcctggacag
601 tgtggagtgt tacgacccag atacagacac ctggagcgag gtgacccgaa tgacatcggg
661 ccggagtggg gtgggcgtgg ctgtcaccat ggagccctgc cggaagcaga ttgaccagca
721 gaactgtacc tgttga
```

By “Nrf2 polypeptide” is meant a protein or protein variant, or fragment thereof, that comprises an amino acid sequence substantially identical to at least a portion of GenBank Accession No. NP_{—}006164 (human nuclear factor (erythroid-derived 2)-like 2) or AAB32188.1 and that has a Nrf2 biological activity (e.g., activation of target genes through binding to antioxidant response element (ARE), regulation of expression of antioxidants and xenobiotic metabolism genes). An exemplary Nrf2 amino acid sequence follows:

```
    1 mdlidilwrq didlgvsrev fdfsqrrkey elekqkklek erqeqlqkeq ekafftqlql
  61 deetgeflpi qpaqhtqset sgsanysqva hipksdalyf ddcmqllaqt fpfvddnevs
121 satfqslvpd ipghiespvf iatnqaqspe tsvaqvapvd ldgmqqdieq vweellsipe
181 lqclniendk lvettmvpsp eakltevdny hfyssipsme kevgncsphf lnafedsfss
241 ilstedpnql tvnslnsdat vntdfgdefy safiaepsis nsmpspatls hslsellngp
301 idvsdlslck afnqnhpest aefndsdsgi slntspsvas pehsvesssy gdtllglsds
361 eveeldsapg svkqngpktp vhssgdmvqp lspsqgqsth vhdaqcentp ekelpvspgh
421 rktpftkdkh ssrleahltr delrakalhi pfpvekiinl pvvdfnemms keqfneaqla
481 lirdirrrgk nkvaaqncrk rklenivele qdldhlkdek ekllkekgen dkslhllkkq
541 lstlylevfs mlrdedgkpy spseyslqqt rdgnvflvpk skkpdvkkn
```

By “Nrf2 nucleic acid molecule” is meant a polynucleotide encoding an Nrf2 polypeptide or variant, or fragment thereof.

By “pulmonary inflammatory condition” is meant any pathological condition that increases mononuclear cells (monocytes/macrophages, lymphocytes), neutrophils, and fibroblasts in the lungs. Exemplary pulmonary inflammatory conditions include, but are not limited to, bacterial, viral, or fungal pulmonary infections, environmental pollutants (e.g., particulate matter, automobile exhaust, allergens), chronic obstructive pulmonary disease, asthma, acute lung injury/acute respiratory distress syndrome or inflammation. The phrase “in combination with” is intended to refer to all forms of administration that provide the inhibitory nucleic acid molecule and the chemotherapeutic agent together, and can include sequential administration, in any order.

The term “subject” is intended to include vertebrates, preferably a mammal. Mammals include, but are not limited to, humans.

By “marker” is meant any protein or polynucleotide having an alteration in expression level or activity that is associated with a disease or disorder.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “inhibitory nucleic acid” is meant a single or double-stranded RNA, siRNA (short interfering RNA), shRNA (short hairpin RNA), or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises or corresponds to at least a portion of a target nucleic acid molecule, or an ortholog thereof, or comprises at least a portion of the complementary strand of a target nucleic acid molecule.

By “antisense nucleic acid”, it is meant a non-enzymatic nucleic acid molecule that binds to target RNA by means of RNA-RNA or RNA-DNA interactions and alters the activity of the target RNA (for a review, see Stein et al. 1993; Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk N A et al., 1999; Delihas N et al., 1997; Aboul-Fadi T, 2005.)

The term “siRNA” refers to small interfering RNA; a siRNA is a double stranded RNA that “corresponds” to or matches a reference or target gene sequence. This matching need not be perfect so long as each strand of the siRNA is capable of binding to at least a portion of the target sequence. SiRNA can be used to inhibit gene expression, see for example Bass, 2001, Nature, 411, 428 429; Elbashir et al., 2001, Nature, 411, 494 498; and Zamore et al., Cell 101:25-33 (2000).

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “obtaining” as in “obtaining the inhibitory nucleic acid molecule” is meant synthesizing, purchasing, or otherwise acquiring the inhibitory nucleic acid molecule.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant protein of the invention, or an RNA molecule).

By “reference” is meant a standard or control condition.

By “restoring corticosteroid responsiveness” is meant increasing the anti-inflammatory action of corticosteroids in subjects having reduced sensitivity to corticosteroid treatment. The restoration need not be complete, but can be an increase in sensitivity of at least about 10%, 25%, 30%, 50%, 75% or more.

By “reversing corticosteroid insensitivity” is meant re-establishing the repressive effect of corticosteroids on cytokine production in subjects having reduced sensitivity to corticosteroid treatment, thereby reducing the levels required for efficacy to those closer to levels typically used in subjects that are not corticosteroid insensitive.

By “respiratory infection” is meant any infection effecting the respiratory system (e.g., lungs and associated tissues). Exemplary respiratory infections include infections with a Gram negative or positive bacteria (e.g., Pseudomonas aeruginosa, nontypeable Haemophilus influenzae, Moraxella catarrhalis, Streptococcus pneumonia, Staphylococcus aureus, or a virus (e.g., Rhinovirus, coronovirus, influenza A and B, parainfluenza, Adenovirus, and Respiratory syncytial virus).

By “promoter” is meant a polynucleotide sufficient to direct transcription.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

The term “pharmaceutically-acceptable excipient” as used herein means one or more compatible solid or liquid filler, diluents or encapsulating substances that are suitable for administration into a human.

By “specifically binds” is meant a molecule (e.g., peptide, polynucleotide) that recognizes and binds a protein or nucleic acid molecule of the invention, but which does not substantially recognize and bind other molecules in a sample, for example, a biological sample, which naturally includes a protein of the invention.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and still more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

“Therapeutic compound” means a substance that has the potential of affecting the function of an organism. Such a compound may be, for example, a naturally occurring, semi-synthetic, or synthetic agent. For example, the test compound may be a drug that targets a specific function of an organism. A test compound may also be an antibiotic or a nutrient. A therapeutic compound may decrease, suppress, attenuate, diminish, arrest, or stabilize the development or progression of disease, disorder, or infection in a eukaryotic host organism.

By “transformed cell” is meant a cell into which (or into an ancestor of which) has been introduced, by means of recombinant DNA techniques, a polynucleotide molecule encoding (as used herein) a protein of the invention.

### BRIEF DESCRIPTION OF THE DRAWINGS

FIGS. 1A-1E are graphs showing that sulforaphane treatment enhances secretion of protease inhibitor, secretory leukocyte protease inhibitor (SLPI) a potent neutrophil elastase inhibitor in alveolar macrophages of patients with COPD. FIG. 1A is a graph of the expression levels of mRNA encoding SLPI. FIG. 1B is a graph of SLPI protein levels in lung lysates as determined by ELISA. FIG. 1C is a graph that compares elastase activity in lung lysates of subjects with advanced COPD and those without COPD. FIG. 1D is a graph of SLPI levels in the culture media of alveolar macrophages with or without sulforaphane treatment. FIG. 1E is a graph of elastase activity in cell free culture media of alveolar macrophages treated with or without sulforaphane.

FIGS. 2A and 2B are graphs showing that sulforaphane treatment by a nebulizer significantly reduced bacterial burden and inflammation in lungs of mice exposed to cigarette smoke (CS) for one week. FIG. 2A is a graph of bacterial burden (PA) in mice treated with sulforaphane after being treated with CS for one week. FIG. 2B is a graph of inflammation in the lungs as assessed by bronchoalveolar lavage in mice immediately after one week of CS exposure. Mice were treated with sulforaphane by nebulizer (0.5 mg/mouse/day) for three consecutive days. Mice were infected with Pseudomonas aeruginosa twenty-four hours after the last dose of sulforaphane.

FIGS. 3A-3D are graphs showing that inoculation of viral ligand Polyl:C causes greater emphysema and inflammation in lungs of mice with deletion of Nrf2 in macrophages and neutrophils (lyzm-Nrf2^{-/-}) when compared to Nrf2 f/f. FIG. 3A is a graph of mean linear intercept which is an indication of emphysema. FIG. 3B is a photomicrograph of lung inflammation as analyzed by histopathological analysis. FIG. 3C is a graph of lung inflammation as analyzed by BAL fluid assay. FIG. 3D is a graph of the type of inflammatory cells in the lungs two weeks after Polyl:C treatment.

FIG. 4 is a Table showing the characteristics of the patients used in the study. Values are presented as mean+/−SEM. Abbreviations: FEV₁ is forced expiratory volume in one second. FVC is forced vital capacity.

FIGS. 5A-5D are graphs showing that sulforaphane, through activation of Nrf2, improves phagocytosis and clearance of Pseudomonas aeruginosa and nontypeable Haemophilus influenzae (NTHI) by COPD alveolar macrophages. FIG. 5A is a plot of Sulforaphane- or vehicle-treated (20 h) COPD alveolar macrophages were incubated with NTHI or PA. Bacterial load in culture medium was quantified after 4 h by serial dilution plating. Data are represented as % of inoculated CFU for individual patient after vehicle or sulforaphane treatment. In FIG. 5B, COPD alveolar macrophages were incubated with attenuated FITC-labeled NTHI or PA after sulforaphane or vehicle treatment and phagocytosed bacteria was quantified by FACS after 1 h. Data are representative histograms from n=5 patients. Representative flow cytometry histogram of an individual patient is presented. In FIG. 5C, COPD alveolar macrophages transfected with Nrf2 siRNA or ssRNA were treated with sulforaphane or vehicle for 20 h. Subsequently, macrophages were incubated with FITC-labeled PA and phagocytosed bacteria was quantified by FACS after 1 h. Data are represented as mean±SEM of MFI; n=3. In FIG. 5D, GSH ester, NAC, sulforaphane- or vehicle-treated alveolar macrophages isolated from patients with COPD were incubated with PA and bacterial load in culture medium was quantified after 4 h. Data are represented as CFUs mean±SEM; n=5 patients.

FIGS. 6A-6G show that Nrf2 regulates scavenger receptor MARCO expression. In FIG. 6A, alveolar macrophages isolated from Keap1^{f/f}, and Lysm-Keap1^{-/-} mice were incubated with PA and bacterial load in culture medium was quantified after 4 hours. Data are represented as CFUs mean±SEM. In FIG. 6B, Basal MARCO mRNA expression levels in BM-DM isolated from Keap1^{f/f} and Lysm-Keap1^{-/-} as measured by microarray analysis. In FIG. 6C, Phagocytosis of FITC-PA in THP-1 macrophages stably transfected with Luciferase shRNA or MARCO shRNA and treated with either vehicle or sulforaphane. FIG. 6D is a plot of Intracellular PA (CFUs) in THP-1 macrophage lysates. Lysates were prepared at varying time periods after sulforaphane or vehicle treatment. Data are from an experiment in triplicate.

FIG. 6E is a schematic of the ChIP assay to determine Nrf2 binding to the promoter region of MARCO gene in macrophages derived from Lysm-Keap1^{-/-} and Keap1^{f/f} mice. FIG. 6F is a set of gels showing the result of a ChIP assay used to determine recruitment of RNA Polll binding to MARCO promoter in macrophages derived from Lysm-Keap1^{-/-} and Keap1^{f/f} mice. * p<0.05.

FIG. 7A-7D are graphs showing that sulforaphane improves bacterial phagocytic function in COPD alveolar macrophages by Nrf2-dependent upregulation of MARCO expression. FIG. 7A is a plot of surface expression of MARCO in COPD alveolar macrophages after sulforaphane or vehicle treatment by FACS analysis. Data are represented as MFI for individual patient with vehicle or sulforaphane treatment. FIG. 7B is a graph of MARCO mRNA expression in COPD alveolar macrophages transfected with mock siRNA, Nrf2 siRNA or no treatment (NT, untransfected) prior to sulforaphane treatment. Data are represented as mean relative fold change (RFC) compared to vehicle treated untransfected cells ±SEM (n=3). * p<0.05. FIG. 7C is a graph of Bacterial (PA) colonization after blocking MARCO receptor by anti-MARCO antibody in sulforaphane-treated alveolar macrophages. Data are represented as mean±SEM of CFUs in culture medium from each treatment (n=3 subjects). FIG. 7D is a graph of FACS analysis of FITC-PA phagocytosis after blocking MARCO receptor by anti-MARCO antibody in sulforaphane-treated alveolar macrophages. Data are represented as mean±SEM of MFI (n=3 subjects). *p<0.05, unless otherwise stated.

FIGS. 8A-8C are graphs showing that cigarette smoke exposure impairs bacterial clearance and enhances inflammation in lungs of mice. In FIG. 8A, alveolar macrophages isolated from wild-type mice exposed to filtered-air or cigarette smoke for 1 week, or 6 months were incubated with FITC-PA and uptake was assessed by FACS. Data are represented as mean±SEM of MFI; n=5/group. * Significant compared to air (p<0.05). FIG. 8B is a graph of CFU's in the culture medium of alveolar macrophages isolated from mice exposed to filtered-air or cigarette smoke (1 week, or 6 months) 4 hours after incubation with PA. Data are represented as mean±SEM of CFU's; n=5/group. * Significant compared to air (p<0.05). FIG. 8C is a graph of bacterial burden in the lungs of mice exposed to filtered-air or cigarette smoke (1 week, or 6 months) 4 h after Pseudomonas aeruginosa infection. Data are represented as mean±SE of CFU's; n=5/group. * Significant compared to air (p<0.05).

FIG. 9 includes four graphs showing that dietary administration of sulforaphane-rich broccoli sprout extract enhances MARCO expression in PBMCs. mRNA expression of Nrf2 target genes in PBMCs isolated from 3 healthy subjects at baseline (pre) and immediately after 2 weeks (post) of daily consumption of broccoli sprout extract (BSE) containing 100 μM of sulforaphane.

FIGS. 10A-10G (S1A-S1G) show that sulforaphane increases Nrf2 protein and activity. FIG. 10A is a graph of Nrf2 protein levels in vehicle or sulforaphane treated COPD alveolar macrophages as measured by flow cytometry. FIG. 10B is a graph of mRNA expression of Nrf2 and Nrf2-regulated antioxidant genes NQO1 and GPX2 in COPD macrophages after sulforaphane treatment. FIG. 10C is a panel of representative cytograms of adherent purified alveolar macrophages obtained from two human subject broncho-alveolar lavage fluid stained for macrophage marker CD14. FIG. 10D is a graph of Pseudomonas aeruginosa colonization in the culture medium of vehicle- or sulforaphane-treated alveolar macrophages from non-COPD patients. FIG. 10E is a graph of Pseudomonas aeruginosa colonization in cell-free medium with or without sulforaphane. FIG. 10F is a plot of mRNA expression of Nrf2 and Nrf2-regulated antioxidant genes NQO1,HO-1 and GPX2 in COPD macrophages transfected with mock siRNA, Nrf2 siRNA or no treatment (NT, untransfected) prior to sulforaphane treatment. FIG. 10G is a plot of intracellular glutathione levels in COPD macrophages after treatment with vehicle, NAC, GSH ester, or sulforaphane.

FIGS. 11A and 11B show that sulforaphane mediated phagocytosis is mediated in part by an increase in MARCO. FIG. 11A is a graph of Pseudomonas aeruginosa colonization (CFU's) in culture medium of THP-1 macrophages treated sulforaphane or vehicle after incubation with poly(I) (10 μg/ml), an scavenger receptor inhibitor. Data represented as CFU's. FIG. 11B is a graph of MARCO expression by FACS in vehicle- or sulforaphane-treated THP-1 cells following stable transfection of luciferase or MARCO shRNA.

FIG. 12 is a western blot of MARCO using PLK-1 antibody in untransfected (NT), luciferase shRNA transfected (Luc shRNA), or MARCO shRNA transfected THP-1 macrophages.

FIG. 13A-13E (S4A-S4B) are graphs showing that sulforaphane increase MARCO in alveolar macrophages in mice exposed to cigarette smoke. FIG. 13A is a graph of MARCO expression (mRNA) in alveolar macrophages isolated from mice exposed to cigarette smoke or air after treatment with bacteria (PA) or sulforaphane. FIG. 13B is a graph of ex vivo Pseudomonas aeruginosa clearance by vehicle or sulforaphane treated alveolar macrophages isolated from mice exposed to room air, 1 week, or 6 months of CS. FIG. 13C is a graph of ex vivo uptake of FITC-PA by vehicle or sulforaphane treated alveolar macrophages isolated from mice exposed to filtered-air, 1 week, or 6 months of CS. FIG. 13D is a plot of Bacterial colonization and (FIG. 13E) Inflammatory cells in broncho-alveolar fluid at 4 h and 24 h post-infection in control (IgG) mice or mice depleted of neutrophils by intraperitoneal administration of anti-Ly6G antibody.

FIGS. 14A and 14B show that sulforaphane reduces bacterial burden in mice treated with cigarette smoke. FIG. 14A is a graph of bacterial burden in lungs of CS (6 months) or air-exposed wild-type mice treated with sulforaphane or vehicle 4 h after Pseudomonas aeruginosa infection. Data are represented as mean±SEM of CFUs; n=5/group. * Significant compared to air or CS alone (p<0.05). FIG. 14B is a graph of bacterial burden in the lungs of CS-exposed mice treated with control (IgG) or anti-Ly6G antibody following vehicle or sulforaphane administration. * Significant compared to CS alone (p<0.05). * Significant compared to vehicle (p<0.05).

FIGS. 15A and 15B show the generation and characterization of Lysm-Keap1^{-/-} conditional knockout mice. FIG. 15A shows specific recombination of the conditional Keap1 allele in the LysM-Keap1^{-/-} mice lungs, liver, kidney, spleen, bone marrow macrophages, and neutrophils. The 288 bp band represents exons 2 and 3 deleted Keap1 allele and 2954 bp band represents the floxed or the wild-type allele. No deletion was detected in the macrophages and neutrophils from Keap1^{f/f} mice. FIG. 15B is a graph of mRNA expression by qRT-PCR of Keap1, Gclm, and Nqo1 genes in bone marrow macrophages and peritoneal neutrophils from Keap1^{-/-} and Keap1^{f/f} mice. *p<0.05.

FIGS. 16A-16G show that sulforaphane increases Nrf2-dependent antioxidant defenses and improves corticosteroid responsiveness in alveolar macrophages. FIG. 16A is an immunoblot analysis of Nrf2 protein in nuclear extracts of alveolar macrophagestreated with sulforaphane or vehicle (VEH) (n=3 patients) and FIG. 16B shows densitometry analysis of immunoblot normalized to lamin B. Data represented as mean±S.D., arbitrary units (AU) from n=9 patients. FIG. 16C is a graph of mRNA analysis of Nrf2-regulated antioxidant genes in alveolar macrophages (n=9 patients). FIG. 16D is a graph of GSH levels in alveolar macrophages after vehicle or sulforaphane treatment (n=9 patients). FIG. 16E is a graph of basal and LPS-induced secretory levels of IL-8 by sulforaphane- or vehicle-treated alveolar macrophages after incubation with or without dexamethasone (DEX) (N=25 patients). FIGS. 16F and 16G show that basal and LPS-induced histone acetylation in the promoter of the IL-8 gene in sulforaphane or vehicle exposed alveolar macrophages after incubation with or without DEX according to ChIP analysis shown with a representative CHIP-PCR blot (n=1) (FIG. 16F) densitometry quantification using n=9 patient samples (FIG. 16G). *Significant compared with vehicle samples by ANOVA analysis followed by Bonferroni post-test; †significant between SUL- and DEX-treated samples with or without LPS treatment; and ‡significant between the two compared samples shown with the arrows, using student's t-test. Data represent mean±s.d; P<0.01.

FIG. 17A-17F shows that sulforaphane improves corticosteroid sensitivity by increasing histone deacetylase 2 (HDAC2) activity in an Nrf2-dependent manner. FIG. 17A is a graph of Total HDAC enzymatic activity in alveolar macrophages treated with or without sulforaphane. FIG. 17B is a graph of HDAC2 enzymatic activity in alveolar macrophages treated with or without sulforaphane. FIG. 17C shows nuclear levels of HDAC2 protein in alveolar macrophages with or without sulforaphane in a representative immunoblot (n=3) and FIG. 17D is a plot of densitometry quantification with patient samples (n=9). FIG. 17E is a graph of basal and LPS-induced histone acetylation in the promoter of the IL-8 gene in AMs, which was coexposed to sulforaphane and TSA, in the presence and absence of DEX. FIG. 17F is gel showing a ChIP analysis of HDAC2 binding and histone acetylation in the IL-8 promoter of AMs, after cotreatment with sulforaphane and TSA or buthionine sulfoximine (BSO), in the presence and absence of DEX. *Significant compared with vehicle samples by ANOVA analysis followed by Bonferroni post-test; †significant between genotypes; and ‡significant between the two compared samples shown with the arrows, using student's t-test. Data represent mean±s.d.; P<0.01. N=25 patient samples, unless otherwise stated for human AMs; n=6 mice/group.

FIG. 18A-18I shows a decrease in posttranslational modification of HDAC2 with concomitant increase in HDAC2 protein in alveolar macrophages after sulforaphane treatment. FIG. 18A is gel showing levels of tyrosine nitration (NO-Tyr HDAC2), serine-phosphorylation (P-Ser HDAC2), and ubiquitination (Ub-HDAC2) in HDAC2 in alveolar macrophages exposed to sulforaphane (SUL) or vehicle according to immunoblot analysis. FIG. 18B is a graph of the ratio of HDAC2 posttranslational modification and total HDAC2 quantified by means of densitometric analysis of the immunoblot from three independent immunoblots (total n=9 patients). FIGS. 18C-18F shows the results of analysis of S-nitrosylation (S-NO) of HDAC2 in alveolar macrophages by anti-SNO antibody (FIG. 18C); densitometric analysis of immunoblot (FIG. 18D), DAN assay (FIG. 18E), and biotin-switch assay (FIG. 18F). FIGS. 18G and 18H show the results of analysis of nitrosative HDAC2 modification and total HDAC2 in peripheral lung tissues of patients with COPD and non-COPD normals (FIG. 18G) and densitometric assessment of total HDAC2 (FIG. 18H). FIG. 181 is a graph of HDAC2 enzymatic activity in peripheral lung tissues (normal [n=9] and COPD [n=9]). *Significant compared with vehicle control or compared with control tissue samples in peripheral lung tissues. Data represent mean±s.d.; P<0.01.

FIGS. 19A-19C shows that L-NAME failed but coexposure to L-NAME and sulforaphane showed an additive effect in restoring corticosteroid sensitivity in AMs. FIG. 19A is a gel showing the levels of NO-Tyr-HDAC2, SNO-HDAC2, and total HDAC2 in alveolar macrophages after treatment with vehicle, sulforaphane, and/or L-NAME. Immunoblot data shown for 2 representative patients but, a total of 6 patients were analyzed. FIG. 19B is a graph of HDAC2 enzymatic activity in alveolar macrophages after treatment with vehicle, sulforaphane, and/or L-NAME. FIG. 19C is a graph of basal and LPS-induced secretory levels of IL-8 by vehicle-, sulforaphane-, and/or L-NAME-treated alveolar macrophages after incubation with or without dexamethasone (n=6/group). *Significant compared with vehicle samples by ANOVA analysis followed by Bonferroni post-test; † or ‡significant between the two compared samples shown with the arrows, using student's t-test. Data represent mean±s.d.; P<0.01.

FIG. 20A-20K show that sulforaphane increases HDAC2 activity by denitrosylation of HDAC2 via GSH. FIGS. 20A and 20B are graphs of the levels of nitrosylation according to a DAN assay (FIG. 20A) and HDAC2 enzymatic activity (FIG. 20B) in cell lysates incubated with GSH in a cell-free reaction (n=6/group). (c-d) FIGS. 20C and 20D are graphs of the analysis of nitrosylation by means of a DAN assay (FIG. 20C) and enzymatic activity (FIG. 20D) in vehicle- or to S-nitrosoglutathione (GSNO)-exposed IP-HDAC2 after incubation with GSH in a cell-free reaction (n=6/group). FIG. 20E is a graph of enzymatic activity of rHDAC2 in the presence or absence of GSH after GSNO treatment (n=6/group). FIG. 20F is a graph of enzymatic activity of HDAC2 in GSNO-exposed alveolar macrophages after GSH-e treatment (n=6/group). FIG. 20G is an immunoblot analysis of NO-Tyr HDAC2 and SNO-HDAC2 in GSNO-exposed AMs. FIG. 20H shows an analysis of SNO-HDAC2 by means of biotin-switch assay in GSNO-exposed alveolar macrophages after GSH-e treatment. FIG. 201 is a graph of enzymatic activity of HDAC2 in GSNO-exposed alveolar macrophages after sulforaphane (SUL) treatments (n=6/group). FIG. 20J is an immunoblot analysis of NO-Tyr HDAC2 and SNO-HDAC2 in GSNO-exposed AMs. FIG. 20K is an analysis of SNO-HDAC2 by means of biotin-switch assay in GSNO-exposed alveolar macrophages after sulforaphane treatment in the presence of BSO. *Significant compared with vehicle samples by ANOVA analysis followed by Bonferroni post-test; ‡significant between the two compared samples shown with the arrows, using student's t-test. Data represent mean±s.d.; P<0.01. All experiments were independently repeated three times.

FIG. 21A-21F and 21H show the Nrf2-dependent restoration of HDAC2 activity with reversal of S-nitrosylation modification on HDAC2 protein in CS-exposed mouse lungs by sulforaphane. Total HDAC (FIG. 21A) and HDAC2 (FIG. 21B) enzymatic activities (n=6 mice/sample). Total nuclear HDAC2 protein representative blot (FIG. 21C) and densitometry assessment (FIG. 21D) for n=6 total mice lung samples/group. FIG. 21E is a graph of total glutathione levels in lung lysates (n=6 mice/sample). Analysis of S-nitrosylation (S-NO) levels of HDAC2 protein by biotin-switch (FIG. 21F) or DAN assays in lung lysates from CS-exposed mice (n=6 mice/sample). FIG. 21H shows the S-nitrosylation of HDAC2 in CSC-exposed peritoneal macrophages after sulforaphane treatment according to biotin-switch assay. Immunoblot data shown for a representative sample but, a total of 6 mice samples were analyzed. *Significant compared with vehicle samples by ANOVA analysis followed by Bonferroni post-test; †significant between genotypes; and ‡significant between the two compared samples shown with the arrows, using student's t-test. Data represent mean±s.d.; P<0.01. Total number of samples, n=6 samples if not otherwise mentioned.

FIG. 22 is a Table showing the characteristics of patients with COPD in the alveolar macrophage analysis.

FIG. 23 is a Table showing the characteristics of patients with COPD in the peritoneal lung tissue assays.

FIGS. 24A-24D show that sulforaphane fails to restore corticosteroid sensitivity in alveolar macrophages from patients with COPD in presence of BSO. FIGS. 24A-24C are graphs of LPS-induced levels of IL-8 mRNA (FIG. 24A); histone acetylation in the promoter of the IL-8 gene (FIG. 24B); and IL-8 protein (FIG. 24C) in alveolar macrophages co-treated with sulforaphane and/or BSO. FIG. 24D is a graph of GSH levels in COPD alveolar macrophages exposed to sulforaphane, and/or BSO after LPS challenge. *Significant compared with vehicle samples by ANOVA analysis followed by Bonferroni post-test; †significant between SUL- and DEX-treated samples with or without LPS treatment; and ‡significant between the two compared samples shown with the arrows, using student's t-test. Data represent mean±s.d; P<0.01. Total number of samples, n=12/group.

FIG. 25 shows that sulforaphane treatment has no effect on HDAC2 mRNA expression. mRNA levels of HDAC2 in alveolar macrophages from patients with COPD (n=12/group) after sulforaphane treatment. Data represent mean±s.d.

FIGS. 26A-26D shows that proteasomal inhibition increase HDAC2 protein but not enzymatic activity in alveolar macrophages from patients with COPD. FIGS. 26A and 26B show total HDAC2 protein levels in alveolar macrophages from patients with COPD as shown with a representative blot (FIG. 26A) for n=4 samples/group, and densitometry quantification (FIG. 26B) on total n=12 samples/group. FIG. 26C is a graph of HDAC2 activity in alveolar macrophages (n=12/group). FIG. 26D is a graph of IL-8 promoter acetylation (n=12/group). *Significant compared with vehicle samples by ANOVA analysis followed by Bonferroni post-test. Data represent mean±s.d.; P<0.01.

FIGS. 27A and 27B show that sulforaphane inhibits NO generation. Levels of NO (FIG. 27A) and iNOS (FIG. 27B) expression in alveolar macrophages from patients with COPD after sulforaphane treatment (n=12 samples/group). *Significant compared with vehicle samples by ANOVA analysis followed by Bonferroni post-test. *Significant compared with vehicle samples by ANOVA analysis followed by Bonferroni post-test; †significant between genotypes in mice samples; ‡significant between sulforaphane- and vehicle-treated mice samples under CSC exposure using student's t-test. Data represent mean±s.d.; P<0.01.

FIG. 28 shows that co-incubation of sulforaphane and L-NAME restores dexamethasone repressive effect on LPS induced IL-8 expression on COPD AMs. Basal and LPS-induced IL-8 mRNA levels in alveolar macrophages treated with vehicle, sulforaphane, and/or NO-inhibitor, L-NAME. *Significant compared with vehicle samples; 1 significant amongst sulforaphane-treated samples by ANOVA analysis followed by Bonferroni post-test. Data represent mean±s.d.; P<0.01.

FIG. 29 shows that GSH-e restores repressive effect of corticosteroids on LPS-induced inflammatory response in to S-nitrosoglutathione (GSNO) exposed COPD AMs. LPS induced secretory levels of IL-8 in GSNO exposed COPD alveolar macrophages after co-treatment with GSH-e or DEX. *Significant compared with vehicle samples by ANOVA analysis followed by Bonferroni post-test; † or ‡significant between the two compared samples shown with the arrows, using student's t-test. Data represent mean±s.d.; P<0.01.

FIGS. 30A and 30B show the deacetylation of glucocorticoid receptor (GR). FIG. 30A shows the levels of deacetylated glucocorticoid receptor (GR) (immunopurified from alveolar macrophages treated with or without dexamethasone) after incubation with immunopurified HDAC2. HDAC2 was immunopurified from GSNO exposed COPD alveolar macrophages after GSH-e treatment. FIG. 30B shows acetylated and total GR protein levels in vehicle- or sulforaphane-treated alveolar macrophages from patients with COPD.

FIGS. 31A-31C show the global S-nitrosylation in AMs. FIGS. 31A and 31B show levels of S-nitrosylated protein in alveolar macrophages after sulforaphane treatment as analyzed by immunoblot analysis with anti-SNO antibody (FIG. 31A) and DAN assay (FIG. 31B). FIG. 31C shows the levels of S-nitrosylated MMP9 (pro and active form) in alveolar macrophages after sulforaphane treatment as analyzed by biotin-switch assay. *Significant compared with vehicle control, data represent mean±s.d.; P<0.01.

### DETAILED DESCRIPTION OF THE INVENTION

The invention generally features therapeutic compositions and methods useful for the treatment of corticosteroid resistance and respiratory infections associated with chronic obstructive pulmonary disease (COPD) and other pulmonary inflammatory conditions.

Corticosteroid resistance due to inactivation of histone deacetylase (HDAC) 2 is a barrier to effective treatment of chronic obstructive pulmonary disease (COPD). As reported in more detail below, the invention is based, at least in part, on the discovery that S-nitrosylation is a key posttranslational modification responsible for inactivation of HDAC2 in COPD alveolar macrophages that can be reversed by targeting transcription factor nuclear factor erythroid 2-related factor 2 (Nrf2). Sulforaphane, a small-molecule activator of Nrf2, restores the function of HDAC2 by denitrosylation in a glutathione-dependent manner, thereby augmenting deacetylation of histones in the interleukin-8 promoter and glucocorticoid receptor in alveolar macrophages from patients with COPD. In contrast to nitric oxide synthase inhibition alone, sulforaphane treatment reestablishes the repressive effect of corticosteroid on cytokine production in alveolar macrophages from patients with COPD. Sulforaphane restores HDAC2 function and corticosteroid sensitivity in alveolar macrophages from cigarette smoke-exposed mice. Thus, Nrf2 is a novel drug target to reverse corticosteroid resistance in COPD and other corticosteroid-resistant inflammatory diseases (e.g., severe asthma, acute graft-versus host disease, autoimmune inner ear disease, inflammatory bowel diseases, and rheumatoid arthritis).

Patients with chronic obstructive pulmonary disease (COPD) have pulmonary innate immune dysfunction largely due to defective macrophage phagocytic ability by unknown mechanisms. This results in periodic bacterial infection and colonization that cause acute exacerbation of COPD, a major source for morbidity and mortality. The invention is further based, at least in part, on the discovery that activation of transcription factor Nrf2 by sulforaphane treatment restores bacterial recognition, phagocytic ability and clearance of clinical isolates nontypeable Haemophilus influenza (NTHI) and Pseudomonas aeruginosa (PA) by alveolar macrophages from patients with COPD. Molecular studies reveal Nrf2 improves macrophage phagocytic ability by direct transcriptional upregulation of class A scavenger receptor MARCO and was independent of it's antioxidant function. Sulforaphane treatment restored phagocytic ability of alveolar macrophages by increasing MARCO and inhibited bacterial colonization (NTHI or PA) and inflammation in the lungs of wild-type mice after 6 months of chronic exposure to cigarette smoke. These findings identify increasing MARCO by targeting Nrf2 as a therapeutic approach to improve anti-bacterial defenses and suggest that this pathway can be targeted for preventing bacterial exacerbations in COPD using agents such as sulforaphane.

Accordingly, agents that increase the expression or biological activity of Nfr2 (e.g., sulforaphane and derivatives or analogs thereof) are useful for reversing corticosteroid resistance, as well as for the treatment of respiratory infections, particularly those associated with chronic obstructive pulmonary disease, emphysema, and related conditions. Therefore, the invention provides compositions for reversing corticosteroid resistance that comprise an agent that increases Nrf2 activity, alone or in combination with a corticosteroid (e.g., dexamethasone, flunisolide, fluticasone propionate, triamcinolone acetonide, beclomethasone dipropionate, budesonide, prednisone, prednisolone, and methylprednisolone). In other embodiments, the invention provides compositions for the treatment of a bacterial infection, particularly for bacterial infections that occur in a subject having or at risk of developing COPD, in subjects having chronic bronchitis, in smokers, and in subjects having cystic fibrosis or having an immunodeficiency syndrome that reduces or otherwise compromises the efficacy of the subject's immune system.

Nuclear Factor E2p45-Related Factor (Nrf2)

Nuclear factor erythroid-2 related factor 2 (NRF2), a cap-and-collar basic leucine zipper transcription factor, regulates a transcriptional program that maintains cellular redox homeostasis and protects cells from oxidative insult (Rangasamy T, et al., J Clin Invest 114, 1248 (2004); Thimmulappa R K, et al. Cancer Res 62, 5196 (2002); So H S, et al. Cell Death Differ (2006)). NRF2 activates transcription of its target genes through binding specifically to the antioxidant-response element (ARE) found in those gene promoters. The NRF2-regulated transcriptional program includes a broad spectrum of genes, including antioxidants, such as γ-glutamyl cysteine synthetase modifier subunit (GCLm), γ-glutamyl cysteine synthetase catalytic subunit (GCLc), heme oxygenase-1, superoxide dismutase, glutathione reductase (GSR), glutathione peroxidase, thioredoxin, thioredoxin reductase, peroxiredoxins (PRDX), cysteine/glutamate transporter (SLC7A11), phase II detoxification enzymes [NADP(H) quinone oxidoreductase 1 (NQO1), GST, UDP-glucuronosyltransferase (Rangasamy T, et al. J Clin Invest 114: 1248 (2004); Thimmulappa R K, et al. Cancer Res 62: 5196 (2002)), and several ATP-dependent drug efflux pumps, including MRP1, MRP2 (Hayashi A, et al. Biochem Biophy Res Commun 310: 824 (2003)); Vollrath V, et al. Biochem J (2006)); Nguyen T, et al. Annu Rev Pharmacol Toxicol 43: 233 (2003)).

### KEAP1

KEAP1 is a cytoplasmic anchor of NRF2 that also functions as a substrate adaptor protein for a Cul3-dependent E3 ubiquitin ligase complex to maintain steady-state levels of NRF2 and NRF2-dependent transcription (Kobayashi et al., Mol Cell Biol 24: 7130 (2004); Zhang D D et al. Mol Cell Biol 24: 10491 (2004)). The Keap1 gene is located at human chromosomal locus 19p13.2. The KEAP1 polypeptide has three major domains: (1) an N-terminal Broad complex, Tramtrack, and Bric-a-brac (BTB) domain; (2) a central intervening region (IVR); and (3) a series of six C-terminal Kelch repeats (Adams J, et al. Trends Cell Biol 10:17 (2000)). The Kelch repeats of KEAP1 bind the Neh2 domain of NRF2, whereas the IVR and BTB domains are required for the redox-sensitive regulation of NRF2 through a series of reactive cysteines present throughout this region (Wakabayashi N, et al. Proc Natl Acad Sci USA 101: 2040 (2004)). KEAP1 constitutively suppresses NRF2 activity in the absence of stress. Oxidants, xenobiotics and electrophiles hamper KEAP1-mediated proteasomal degradation of NRF2, which results in increased nuclear accumulation and, in turn, the transcriptional induction of target genes that ensure cell survival (Wakabayashi N, et al. Nat Genet 35: 238 (2003)). Germline deletion of the KEAP1 gene in mice results in constitutive activation of NRF2 (Wakabayashi N, et al Nat Genet 35: 238 (2003)). Recently, a somatic mutation (G430C) in KEAP1 in one lung cancer patient and a small-cell lung cancer cell line (G364C) have been described (Padmanabhan B, et al. Mol Cell 21: 689 (2006)). Prothymosin α, a novel binding partner of KEAP1, has been shown to be an intranuclear dissociator of NRF2-KEAP1 complex and can upregulate the expression of Nrf2 target genes (Karapetian R N, et al. Mol Cell Biol 25: 1089 (2005)).

### Nrf2 Activating Agents

Given that increased Nrf2 expression or activity is useful for the treatment or prevention of corticosteroid resistance, as well as for the treatment of respiratory infections, particularly those associated with chronic obstructive pulmonary disease, emphysema, and related pulmonary inflammatory conditions, agents that activate Nrf2 are useful in the methods of the invention. Such agents are known in the art and are described herein. Exemplary Nrf2 activating compounds include sulforaphane, and analogs or derivatives thereof, as well as those agents described in U.S. Patent Publication No. 2004/002463. Other exemplary Nrf2 activating agents are listed in Table 1.

TABLE 1

| Nrf2 activator | Year | Reference |
|---|---|---|
| 1,2,3,4,6-Penta-O-Galloyl- | 2006 | Mol Pharmacol. 2006 May; 69(5): 1554-63. Epub 2006 |
| Beta-D-Glucose | | Jan 31. |
| 1,2-Diphenol (Catechol) | 2000 | J Biol Chem, Vol. 275, Issue 15, 11291-11299, Apr. 14, |
| | | 2000 |
| 1,2-Dithiole-3-Thione | 2002 | J Biol Chem. 2003 Jan 10; 278(2): 703-11. Epub 2002 Oct |
| | | 4. |
| 1,4-Diphenols (P- | 2000 | J Biol Chem, Vol. 275, Issue 15, 11291-11299, Apr. 14, |
| Hydroquinone) | | 2000 |
| 15-Deoxy-12,14-Pgj2 | 2000 | J Biol Chem, Vol. 275, Issue 15, 11291-11299, Apr. 14, |
| | | 2000 |
| 2-Indol-3-Yl- | 2003 | Biochem Biophys Res Commun. 2003 Aug |
| Methylenequinuclidin-3- | | 8; 307(4): 973-9. |
| Ols | | |
| 3-Hydroxyanthranilic Acid | 2006 | Drug Metab Dispos. 2006 Jan; 34(1): 152-65. Epub 2005 |
| | | Oct 21. |
| 3-Methylcholanthrene | 2006 | Febs J. 2006 Jun; 273(11): 2345-56. |
| 4-Hydroxyestradiol | 2003 | Mol Cell Biol. 2003 Oct; 23(20): 7198-209. |
| 4-Hydroxynonenal | 2005 | J Immunol. 2005 Oct 1; 175(7): 4408-15. |
| 6-Methylsulfinylhexyl | 2002 | J. Biol. Chem., Vol. 277, Issue 5, 3456-3463, Feb. 1, |
| Isothiocyanate | | 2002 |
| 7-Oh Cmrn | 2001 | Cancer Research 61, 3299-3307, Apr. 15, 2001 |
| 9-Cis-Retinoic Acid | 2004 | Proc Natl Acad Sci USA. 2004 Mar 9; 101(10): 3381-6. |
| | | Epub 2004 Feb 25. |
| Acetylcarnitine | 2004 | J Nutr. 2004 Dec; 134(12 Suppl): 3499s-3506s |
| Allyl Isothiocyanate | 2005 | J Invest Dermatol. 2005 Apr; 124(4): 825-32. |
| Alpha-Lipoic Acid | 2005 | Chem Res Toxicol. 2005 Aug; 18(8): 1296-305. |
| Apomorphine | 2006 | Ann N Y Acad Sci. 2006 May; 1067: 420-4. |
| AUR ((2,3,4,6-Tetra-O)- | 2001 | J. Biol. Chem., Vol. 276, Issue 36, 34074-34081, |
| Acetyl-1-Thio-D- | | Sep. 7, 2001 |
| Glucopyranosato- | | |
| S)(Triethylphosphine) | | |
| Gold(I) | | |
| Autg ((1-Thio-D- | 2001 | J. Biol. Chem., Vol. 276, Issue 36, 34074-34081, |
| Glucopyranosato) Gold(I) | | Sep. 7, 2001 |
| Autm (Sodium | 2001 | J. Biol. Chem., Vol. 276, Issue 36, 34074-34081, |
| Aurothiomalate | | Sept. 7, 2001 |
| Avicins | 2004 | J Biol Chem. 2004 Mar 5; 279(10): 8919-29. Epub 2003 |
| | | Dec 19. |
| Bis(2- | 2006 | Cell Death Differ. 2006 Feb 17 |
| Hydroxybenzylidene)Acetone | | |
| B-Naphthoflavone | 1998 | Oncogene (1998) 17, 3145 ± 3156 |
| Broccoli Seeds | 2004 | Free Radic Biol Med. 2004 Nov 15; 37(10): 1578-90. |
| Bucillamine | 2006 | Biomaterials. 2006 Jun 24; |
| Butylated Hydroxyanisole | 1997 | Biochemical And Biophysical Research Communications |
| | | 236, 313-322 (1997) |
| Butylated Hydroxytoulene | 1999 | PNAS U Oct. 26, 1999 U Vol. 96 U No. 22 U |
| | | 12731-12736 |
| Cafestol | 2001 | Cancer Research 61, 3299-3307, Apr. 15, 2001 |
| Carnosol | 2004 | J Clin Invest. 2004 Nov; 114(9): 1248-59. |
| Catechol | 2000 | J Biol Chem. 2000 May 26; 275(21): 16023-9. |
| Chlorogenic Acid | 2005 | Cancer Res. 2005 Jun 1; 65(11): 4789-98. |
| Coumarin | 2001 | Cancer Research 61, 3299-3307, Apr. 15, 2001 |
| Curcumin | 2003 | J Biol Chem. 2003 Feb 14; 278(7): 4536-41. Epub 2002 |
| | | Nov 22. |
| Deprenyl (Selegiline) | 2006 | Carcinogenesis. 2006 May; 27(5): 1008-17. Epub 2005 |
| | | Nov 23. |
| Dexamethasone 21- | 2002 | Toxicol Lett. 2002 Jun 7; 132(1): 27-36. |
| Mesylate | | |
| Diallyl Disulfide | 2004 | Free Radic Biol Med. 2004 Nov 15; 37(10): 1578-90. |
| Diallyl Sulfide | 2004 | Febs Lett. 2004 Aug 13; 572(1-3): 245-50. |
| Diallyl Trisulfide (DATS) | 2004 | Free Radic Biol Med. 2004 Nov 15; 37(10): 1578-90. |
| Epicatechin-3-Gallate | 2001 | Drug Metabolism Reviews Volume 33, Number 3-4/ |
| | | 2001 |
| Epigallocatechin-3-Gallate | 2005 | Arterioscler Thromb Vasc Biol. 2005 Oct; 25(10): 2100-5. |
| | | Epub 2005 Aug 25. |
| Eriodictyo | 2006 | Invest Ophthalmol Vis Sci. 2006 Jul; 47(7): 3164-77. |
| Ethoxyquin. | 2000 | Biochem Soc Trans. 2000 Feb; 28(2): 33-41. |
| Ferulic Acid (Trans-4- | 2006 | Carcinogenesis. 2006 May; 27(5): 1008-17. Epub 2005 |
| Hydroxy-3- | | Nov 23. |
| Methoxycinnamic Acid, | | |
| 99% Purity) | | |
| Fisetin | 2006 | Invest Ophthalmol Vis Sci. 2006 Jul; 47(7): 3164-77. |
| Flunarizine | 2006 | World J Gastroenterol. 2006 Jan 14; 12(2): 214-21. |
| Gallic Acid (3,4,5- | 2006 | Carcinogenesis. 2006 May; 27(5): 1008-17. Epub 2005 |
| Trihydroxybenzoic Acid, | | Nov 23. |
| Gentisic Acid | 2006 | Carcinogenesis. 2006 May; 27(5): 1008-17. Epub 2005 |
| | | Nov 23. |
| Glucose Oxidase | 2000 | J Biol Chem. 2000 May 26; 275(21): 16023-9. |
| Glycosides From Digitalis | 2006 | Food Chem Toxicol. 2006 Aug; 44(8): 1299-307. Epub |
| Purpurea | | 2006 Mar 6. |
| Heme | 1999 | J. Biol. Chem., Vol. 274, Issue 37, 26071-26078, |
| | | Sep. 10, 1999 |
| Hemin | 2001 | J Biol Chem. 2001 May 25; 276(21): 18399-406. Epub |
| | | 2001 Mar 1. |
| Hyerpoxia | 2005 | Free Radic Biol Med. 2005 Feb 1; 38(3): 325-43. |
| Indole-3-Carbinol | 2001 | Cancer Research 61, 3299-3307, Apr. 15, 2001 |
| Indomethacin | 2002 | Drug Metabolism Reviews Volume 33, Number 3-4/ |
| | | 2002 |
| Insulin | 2006 | Pharmazie. 2006 Apr; 61(4): 356-8. |
| Iodoacetic Acid | 2000 | J Biol Chem. 2000 May 26; 275(21): 16023-9. |
| Kahweol Palmitate | 2001 | Cancer Research 61, 3299-3307, Apr. 15, 2001 |
| Limettin (LMTN) | 2001 | Cancer Research 61, 3299-3307, Apr. 15, 2001 |
| Lipoic Acid. | 2004 | J Clin Invest. 2004 Jan; 113(1): 65-73. |
| Luteolin | 2006 | J Neurosci Res. 2006 Jun 26 |
| Lycopene | 2005 | J Neurosci Res. 2005 Feb 15; 79(4): 509-21. |
| Nitric Oxide-Donating | 2005 | Atherosclerosis. 2005 Oct 20; |
| Aspirin | | |
| Oltipraz | 2001 | Proc Natl Acad Sci USA. 2001 Mar 13; 98(6): 3410-5 |
| Parthenolide | 2005 | J Biochem Mol Biol. 2005 Mar 31; 38(2): 167-76. |
| P-Coumaric Acid (Trans-4- | 2006 | Carcinogenesis. 2006 May; 27(5): 1008-17. Epub 2005 |
| Hydroxycinnamic Acid), | | Nov 23. |
| Pgj2 | 2000 | J Biol Chem, Vol. 275, Issue 15, 11291-11299, Apr. 14, |
| | | 2000 |
| Phenethyl Isothiocyanate | 2003 | Biochim Biophys Acta. 2003 Oct 1; 1629(1-3): 92-101. |
| Phorbol 12-Myristate 13- | 2000 | Proc Natl Acad Sci USA. 2000 Nov 7; 97(23): 12475-80. |
| Acetate (PMA) | | |
| P-Hydroxybenzoic Acid | 2006 | Carcinogenesis. 2006 Apr; 27(4): 803-10. Epub 2005 Nov |
| | | 2. |
| Proteasome Inhibitor MG- | 2006 | Biochem Pharmacol. 2006 Jun 24; |
| 132 | | |
| Proteasome Inhibitors | 2003 | J Biol Chem. 2003 Jan 24; 278(4): 2361-9. Epub 2002 |
| (Lactacystin Or MG-132) | | Nov 14. |
| Pyrrolidine | 2003 | Circ Res. 2003 Mar 7; 92(4): 386-93. Epub 2003 Feb 6. |
| Dithiocarbamate | | |
| Quercetin | 2006 | Invest Ophthalmol Vis Sci. 2006 Jul; 47(7): 3164-77. |
| Quercetin 3-O-Beta-L- | 2006 | Biochem Biophys Res Commun. 2006 May |
| Arabinopyranoside | | 12; 343(3): 965-72. Epub 2006 Mar 29. |
| Spermidine | 2003 | Toxicol Sci. 2003 May; 73(1): 124-34. Epub 2003 Mar 25. |
| Spermine | 2003 | Biochem Biophys Res Commun. 2003 Jun 6; 305(3): 662-70. |
| Spermine Nonoate | 2003 | Biochem Biophys Res Commun. 2003 Jun 6; 305(3): 662-70. |
| Sulforaphane | 2002 | Cancer Res. 2002 Sep 15; 62(18): 5196-203. |
| Sulforaphane | 2002 | J. Biol. Chem., Vol. 277, Issue 5, 3456-3463, Feb. 1, |
| | | 2003 |
| Trans-Stilbene Oxide | 2006 | Biochem Biophys Res Commun. 2006 Jan |
| | | 20; 339(3): 915-22. Epub 2005 Nov 28. |
| Wasabi Extract | 2001 | Toxicol Appl Pharmacol. 2001 Jun 15; 173(3): 154-60. |
| Xanthohumol (XH) | 2005 | Biochem J. 2005 Oct 15; 391(Pt 2): 399-408. |
| Zerumbone | 2004 | Faseb J. 2004 Aug; 18(11): 1258-60. Epub 2004 Jun 18. |

### Keap1 RNA Interference

Keap1 is a known inhibitor of Nrf2. Agents that reduce Keap1 expression are useful for the treatment of diseases and disorders associated with corticosteroid resistance, as well as for the treatment of respiratory infections, particularly those associated with chronic obstructive pulmonary disease, emphysema, and related pulmonary inflammatory conditions. Keap1 inhibitors may be used alone, or in combination with Nrf2 activating agents, and/or corticosteroids.

RNA interference (RNAi) is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of shRNAs using a plasmid-based expression system is currently being used to create loss-of-function phenotypes in mammalian cells. siRNAs that target Keap1 decrease Keap1 expression thereby activating Nrf2.

### Keap1 Inhibitory Nucleic Acid Molecules

Keap1 inhibitory nucleic acid molecules are essentially nucleobase oligomers that may be employed as single-stranded or double-stranded nucleic acid molecule to decrease Keap1 expression. In one approach, the Keap1 inhibitory nucleic acid molecule is a double-stranded RNA used for RNA interference (RNAi)-mediated knock-down of Keap1 gene expression. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to an Keap1 gene comprises at least a fragment of the double-stranded gene, such that each strand of the double-stranded inhibitory nucleic acid molecule is capable of binding to the complementary strand of the target Keap1 gene. The inhibitory nucleic acid molecule need not have perfect correspondence to the reference Keap1 sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

The inhibitory nucleic acid molecules provided by the invention are not limited to siRNAs, but include any nucleic acid molecule sufficient to decrease the expression of an Keap1 nucleic acid molecule or polypeptide. Each of the DNA sequences provided herein may be used, for example, in the discovery and development of therapeutic antisense nucleic acid molecule to decrease the expression of Keap1. The invention further provides catalytic RNA molecules or ribozymes. Such catalytic RNA molecules can be used to inhibit expression of an Keap1 nucleic acid molecule in vivo. The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

In one embodiment, the inhibitory nucleic acid molecules of the invention are administered systemically in dosages between about 1 and 100 mg/kg (e.g., 1, 5, 10, 20, 25, 50, 75, and 100 mg/kg). In other embodiments, the dosage ranges from between about 25 and 500 mg/m²/day. Desirably, a human patient receives a dosage between about 50 and 300 mg/m²/day (e.g., 50, 75, 100, 125, 150, 175, 200, 250, 275, and 300).

### Modified Inhibitory Nucleic Acid Molecules

A desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. The nucleobase units are maintained for hybridization with an Keap1 nucleic acid molecule. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

### Nrf2 and Keap1 Polynucleotides

In general, the invention includes any nucleic acid sequence encoding an Nrf2 polypeptide or a Keap1 inhibitory nucleic acid molecule. Also included in the methods of the invention are any nucleic acid molecule containing at least one strand that hybridizes with such a Keap1 nucleic acid sequence (e.g., an inhibitory nucleic acid molecule, such as a dsRNA, siRNA, shRNA, or antisense molecule). The Keap1 inhibitory nucleic acid molecules of the invention can be 19-21 nucleotides in length. In some embodiments, the inhibitory nucleic acid molecules of the invention comprises 20, 19, 18, 17, 16, 15, 14, 13, 12, 11, 10, 9, 8, or 7 identical nucleotide residues. In yet other embodiments, the single or double stranded antisense molecules are 60%, 65%, 70%, 75%, 80%, 85%, 90%, 95%, 96%, 97%, 98%, 99% complementary to the Keap1 target sequence. An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

Further embodiments can include any of the above inhibitory polynucleotides, directed to Keap1, Phase II genes, including glutathione-S-transferases (GSTs), antioxidants (GSH), and Phase II drug efflux proteins, including the multidrug resistance proteins (MRPs), or portions thereof.

### Delivery of Nucleobase Oligomers

Naked oligonucleotides are capable of entering tumor cells and inhibiting the expression of Keap1. Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Nrf2 Polynucleotide Therapy

Methods for expressing Nrf2 in a cell of a subject are useful for increasing the expression of downstream target genes. Polynucleotide therapy featuring a polynucleotide encoding a Nrf2 nucleic acid molecule or analog thereof is one therapeutic approach for treating or preventing a disease or disorder associated with corticosteroid resistance, as well as for the treatment of respiratory infections, particularly those associated with chronic obstructive pulmonary disease, emphysema, and related pulmonary inflammatory conditions in a subject. Expression vectors encoding nucleic acid molecules can be delivered to cells of a subject having a disease or disorder associated with corticosteroid resistance, bacterial respiratory infections, particularly those associated with chronic obstructive pulmonary disease. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a Nrf2 nucleic acid molecule, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of an Nrf2 nucleic acid molecule therapeutic to a cell of a patient diagnosed as having a disease or disorder associated with corticosteroid resistance, as well as for the treatment of respiratory infections, particularly those associated with chronic obstructive pulmonary disease. For example, a Nrf2 nucleic acid molecule can be introduced into a cell (e.g., a lung cell, alveolar macrophage) by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the Nrf2 nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell.

Nrf2 nucleic acid molecule expression for use in polynucleotide therapy methods can be directed from any suitable promoter (e.g., the human cytomegalovirus (CMV), simian virus 40 (SV40), or metallothionein promoters), ubiquitin promoter and regulated by any appropriate mammalian regulatory element. In one embodiment, a promoter that directs expression in a pulmonary tissue is used. If desired, enhancers known to preferentially direct gene expression in specific cell types can also be used to direct the expression of a nucleic acid. The enhancers used can include, without limitation, those that are characterized as tissue- or cell-specific enhancers.

For any particular subject, the specific dosage regimes should be adjusted over time according to the individual need and the professional judgment of the person administering or supervising the administration of the compositions.

### Pharmaceutical Compositions

As reported herein, increased Nrf2 expression or biological activity is useful for reversing corticosteroid resistance, as well as for the treatment of respiratory infections (e.g., P. aeruginosa, H. influenza, Rhinovirus, coronovirus, influenza A and B, parainfluenza, Adenovirus, and Respiratory syncytial virus), particularly those associated with chronic obstructive pulmonary disease, emphysema, and related pulmonary inflammatory conditions. Accordingly, the invention provides therapeutic compositions that increase Nrf2 expression in a tissue (e.g., in an alveolar macrophage or in lung tissue). As detailed above, the invention is particularly useful for the treatment of corticosteroid resistance. Thus, in one embodiment an agent that activates Nrf2 expression (e.g., sulforaphane) is administered alone or in combination with a corticosteroid (e.g., dexamethasone, flunisolide, fluticasone propionate, triamcinolone acetonide, beclomethasone dipropionate, budesonide, prednisone, prednisolone, and methylprednisolone). In other embodiments, an agent that increases Nrf2 activity is administered for the treatment of a respiratory infection. The methods and compositions of the invention are particularly useful for the treatment of respiratory infections that occur in subjects that smoke cigarettes, in subjects with chronic bronchitis, and in subject that have COPD.

In another embodiment, an agent that activates Nrf2 expression (e.g., sulforaphane) is administered alone or in combination with an antibiotic. Antibiotics useful in the methods and compositions of the invention include the penicillins (e.g., penicillin G, ampicillin, methicillin, oxacillin, and amoxicillin), the cephalosporins (e.g., cefazolin, cefuroxime, cefotaxime, and ceftriaxone, ceftazidime), the carbapenems (e.g., imipenem, ertapenem, and meropenem), the tetracyclines and glycylclines (e.g., doxycycline, minocycline, tetracycline, and tigecycline), the aminoglycosides (e.g., amikacin, gentamycin, kanamycin, neomycin, streptomycin, and tobramycin), the macrolides (e.g., azithromycin, clarithromycin, and erythromycin), the quinolones and fluoroquinolones (e.g., gatifloxacin, moxifloxacin, sitafloxacin, ciprofloxacin, lomefloxacin, levofloxacin, and norfloxacin), the glycopeptides (e.g., vancomycin, teicoplanin, dalbavancin, and oritavancin), dihydrofolate reductase inhibitors (e.g., cotrimoxazole, trimethoprim, and fusidic acid), the streptogramins (e.g., synercid), the oxazolidinones (e.g., linezolid) and the lipopeptides (e.g., daptomycin).

In one embodiment, the present invention provides a pharmaceutical composition comprising a Keap1 inhibitory nucleic acid molecule (e.g., an antisense, siRNA, or shRNA polynucleotide) that decreases the expression of a Keap1 nucleic acid molecule or polypeptide. If desired, the Keap1 inhibitory nucleic acid molecule is administered in combination with an agent that activates Nrf2 (e.g., sulforaphane) and/or in combination with a corticosteroid. In various embodiments, the Keap1 inhibitory nucleic acid molecule is administered prior to, concurrently with, or following administration of the agent that activates Nrf2 or with the corticosteroid. Without wishing to be bound by theory, administration of a Keap1 inhibitory nucleic acid molecule enhances the biological activity of Nrf2. Polynucleotides of the invention may be administered as part of a pharmaceutical composition. The compositions should be sterile and contain a therapeutically effective amount of the polypeptides or nucleic acid molecules in a unit of weight or volume suitable for administration to a subject.

A nucleic acid molecule encoding Nrf2, an inhibitory nucleic acid molecule of the invention, together with an corticosteroid, may be administered within a pharmaceutically-acceptable diluents, carrier, or excipient, in unit dosage form. Conventional pharmaceutical practice may be employed to provide suitable formulations or compositions to administer the compounds to patients suffering from corticosteroid resistance, as well as for the treatment of respiratory infections, particularly those associated with chronic obstructive pulmonary disease. Administration may begin before the patient is symptomatic. Any appropriate route of administration may be employed, for example, administration may be by inhalation, or parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for an agent that increases Nrf2 activity, nucleic acid molecules encoding Nrf2 or Keap1 inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If particular embodiments, agents of the invention (e.g. sulforaphane and agents that increase Nrf2 expression or activity, alone or in combination with corticosteroids) can be delivered using standard pulmonary drug administration formulations. Administration by this route offers several advantages, for example, rapid onset of action by administering the drug to the desired site of action at higher local concentrations. Pulmonary drug formulations are generally categorized as nebulized and aerosolized formulations, which are each described further, as follows.

Nebulizers employ agents of the invention in droplet form, in solution or suspension, with a pharmaceutically acceptable liquid carrier. Examples of this approach, such as jet nebulization, are described, e.g., in Flament et al., Drug Development and Industrial Pharmacy 21(20):2263-2285, 1995. Briefly, in such methods, air is passed rapidly through a narrow orifice of a tube by the use of a pump, the pressure of the air falls, creating a vacuum, which results in suction of liquid contained in a reservoir connected with the tube. The suctioned liquid is thus reduced to a fine spray or mist that can be inhaled.

When an agent of the invention is applied to the device, the resulting aerosol deliver the agent into the lungs by normal breathing. The small droplets are dispersed throughout the entire surface of the lung, providing a large and broad delivery of the agent. Droplets of this size can be produced using any method known in the art. For example, U.S. Pat. No. 4,533,082, discloses a fluid droplet production apparatus with a membrane and a piezo-electric actuator that contracts and expands in order to drive the membrane.

Nebulizers, in particular, inhalation nebulizers that form aerosols are of particular use in the methods of the invention as described herein. A variety of inhalation nebulizers are known. EP 0170715A1, incorporated by reference in its entirety herein, uses a compressed gas flow to form an aerosol. A nozzle is arranged as an aerosol generator in an atomizer chamber of the inhalation nebulizer and has two suction ducts arranged adjacent a compressed-gas channel. When compressed air flows through the compressed-gas channel, the liquid to be nebulized is drawn in through the suction ducts from a liquid storage container. This nebulizer is an example of continuously operating inhalation nebulizers, in which the aerosol generator produces an aerosol not only during inhalation, but also while the subject exhales. The aerosol produced by the aerosol generator is actually inhaled by the patient only in the inhalation phase, while any aerosol produced at other times is lost. In order to avoid aerosol losses, attempts have been made to restrict aerosol production to part or all of the inhalation phase. Either a patient can interrupt aerosol production manually, or the patient's respiration can be detected by sensors that automatically control aerosol production. Neither situation is flawless, as manual control of aerosol production is an additional strain for patients and often leads to insufficient results. Automatic control of aerosol production represents an enormous technical expenditure which as a rule bears little relation to the obtained benefit.

In the methods of the invention, inhalation nebulizers can be used to deliver therapeutically effective amounts of pharmaceuticals by forming an aerosol which includes particles of a size that can easily be inhaled. The aerosol can be used, for example, by a patient within the bounds of an inhalation therapy, whereby the therapeutically effective agent reaches the patient's respiratory tract upon inhalation.

One such inhalation nebulizer is the PARI device, which is commercially available. PARI's eFlow, an electronic, portable nebulizer, enables aerosolization of liquid medications via a vibrating, perforated membrane. The PARI device is described in U.S. Pat. No. 5,152,456, U.S. Pat. No. 5,261,601, U.S. Pat. No. 5,518,179, U.S. Pat. No. 6,962,151, incorporated by reference in their entireties herein. Information on the PARI device can be found publicly on the world wide web at http://www.paripharma.com/technologies1.htm.

U.S. Pat. No. 5,152,456, incorporated by reference in its entirety herein, describes a dispensing apparatus that comprises a housing defining a chamber receiving liquid to be dispensed and comprising a perforate membrane which defines a front wall of the chamber. A vibrating device is connected to the housing and is operable to vibrate the perforate membrane to dispense droplets of liquid through holes in the perforate membrane. The housing comprises an annular member having a relatively thin inner annular portion connected to the perforate membrane and a relatively thick outer annular portion connected to the vibrating device. The U.S. Pat. No. 5,152,456 patent describes an apparatus that is suitable for dispensing pharmaceutical products as an atomized mist, and provides a hand-held inhaler for oral inhalation.

U.S. Pat. No. 5,261,601, incorporated by reference in its entirety herein, describes a dispensing apparatus for use in dispensing liquid as an atomized spray. The U.S. Pat. No. 5,261,601 describes a dispensing apparatus that comprises a housing defining a chamber receiving liquid to be dispensed and comprising a perforate membrane which defines a front wall of the chamber. A vibrating means is connected to the housing and is operable to vibrate the perforate membrane to dispense droplets of liquid through holes in the perforate membrane. The membrane defines an array of holes each of which is flared such that the cross-section narrows in a direction from the rear surface of the membrane in contact with the liquid towards the front surface of the membrane. The apparatus is suitable for dispensing pharmaceutical products as an atomized mist and provides a hand-held inhaler for oral inhalation.

U.S. Pat. No. 5,518,179, incorporated by reference in its entirety herein, describes a fluid droplet production apparatus, for example, for use an atomizer spraying device, that has a membrane which is vibrated by an actuator, which has a composite thin-walled structure and is arranged to operate in a bending mode. According to a certain embodiments of the invention the fluid droplet apparatus comprises a membrane, an actuator, for vibrating the membrane, where the actuator comprises a composite thin-walled structure arranged to operate in a bending mode and to vibrate the membrane substantially in the direction of actuator bending, and a means for supplying fluid directly to a surface of the membrane, as fluid is sprayed therefrom on vibration of the membrane. The membrane is structured so as to influence the menisci of fluid introduced to the membrane.

In certain preferred embodiments, the actuator is substantially planar, but it is possible that thin-walled curved structures may be appropriate in some circumstances. Another thin-walled structure which is not planar, would be a structure having bonded layers in which the stiffness of each layer varied across the common face area over which they are bonded in substantially the same way. In all cases, the actuator is thin-walled over its whole area. Fluid is brought from a fluid source directly into contact with the membrane (which may be tapered in thickness and/or have a textured surface) and is dispensed from the membrane by the operation of the vibration means, (advantageously without the use of a housing defining a chamber of which the membrane is a part). The membrane may be a perforate membrane, in which case the front face may have annular locally raised regions disposed substantially concentrically with the holes.

Preferably, the holes defined by a perforate membrane each have a relatively smaller cross-sectional area at the front face and a relatively larger cross-sectional area at the rear face, and can be referred to as tapered holes. In certain examples, the reduction in cross-sectional area of the tapered holes from rear face to front face is smooth and monotonic. Such tapered holes are believed to enhance the dispensation of droplets. In response to the displacement of the relatively large cross-sectional area of each hole at the rear face of the perforate membrane a relatively large fluid volume is swept in this region of fluid.

The size of the smaller cross-sectional area of the perforations on the front face of the membrane may be chosen in accordance with the diameter of the droplets desired to be emergent from the membrane. Dependent upon fluid properties and the excitation operating conditions of the membrane, for circular cross-sectional perforation the diameter of the emergent droplet is typically in the range of 1 to 3 times the diameter of the perforation on the droplet-emergent face of the membrane.

Other factors, such as the exact geometrical form of the perforations, being fixed, the degree of taper influences the amplitude of vibration of the membrane needed for satisfactory droplet production from that perforation. Substantial reductions in the required membrane vibrational amplitude are found when the mean semi-angle of the taper is in the range 30 degrees to 70 degrees, although improvements can be obtained outside this range.

For perforate membranes with tapered perforations as described above, it is found that fluid may be fed from the fluid source by capillary feed to a part of the front face of the membrane and in this embodiment fluid is drawn through at least some of the holes in the membrane to reach the rear face of the membrane prior to emission as droplets by the action of the vibration of the membrane by the vibration means. This embodiment has the advantage that, in dispensing fluids that are a multi-phase mixture of liquid(s) and solid particulate components, examples being suspensions and colloids, only those particulates whose size is small enough in comparison to the size of the holes for their subsequent ejection within fluid droplets pass through from the front to the rear face of the perforate membrane. In this way the probability of perforate membrane clogging by particulates is greatly reduced.

The faces of the membrane need not be planar. In particular, for perforate membranes, the front face may advantageously have locally raised regions immediately surrounding each hole. Such locally-raised regions are believed to enhance the dispensation of droplets by more effectively “pinning” the menisci of the fluid adjacent to the front face of the holes than is achieved by the intersection of the holes with a planar front face of the membrane, and thereby to alleviate problems with droplet dispensation caused by “wetting” of the front face of the membrane by the fluid.

Other conditions being fixed, such tapered perforations reduce the amplitude of vibration of the perforated membrane needed to produce droplets of a given size. One reason for such reduction of amplitude being achieved is the reduction of viscous drag upon the liquid as it passes through the perforations. Consequently a lower excitation of the electromechanical actuator may be used. This gives the benefit of improved power efficiency in droplet creation.

One advantage of the arrangement of the invention is that a relatively simple and low cost apparatus may be used for production of a fluid droplet spray.

U.S. Pat. No. 6,962,151, incorporated by reference in its entirety herein, describes an inhalation nebulizer having both an aerosol generator and a mixing chamber. The aerosol generator includes a liquid storage container for a liquid medicament. In this, a liquid medicament can be a drug that is itself a liquid, or the liquid medicament can be a solution, suspension or emulsion that contains the medicament of interest. In a preferred embodiment, the liquid medicament is an active agent that is in a solution, a suspension or an emulsion.

The aerosol generator also includes a diaphragm that is connected on one side to the liquid storage container, such that a liquid contained in the liquid storage container will come into contact with one side of the diaphragm. The diaphragm is connected to a vibration generator that can vibrate the diaphragm so that a liquid in the liquid storage container can be dispensed or dosed for atomization through openings present in the diaphragm and enter the mixing chamber.

The mixing chamber has an inhalation valve that allows ambient air to flow into the mixing chamber during an inhalation phase while preventing aerosol from escaping during an exhalation phase. The mixing chamber also has an exhalation valve that allows discharge of the patient's respiratory air during the exhalation phase while preventing an inflow of ambient air during the inhalation phase.

The aerosol generator may include a cylindrical liquid storage container that is defined on one side by a diaphragm that preferably is a circular disk. A liquid filled in the liquid storage container contacts one side of the diaphragm. A vibration generator, for instance a piezoelectric crystal, surrounds the diaphragm circumferentially such that the diaphragm can be vibrated by the vibration generator. This requires a electric drive circuit for the vibration generator, the structure and function of which are well known to the person skilled in this art. Through openings present in the diaphragm, the liquid adjoining one side of the diaphragm is atomized through to the other side of the diaphragm and thus is atomized into the mixing chamber.

The liquid storage container preferably provides an entry point for the medicament to be dispensed. In one embodiment, the liquid storage container is a liquid reservoir that is directly fitted into the inhalation nebulizer. In another embodiment, the medicament is provided to the liquid storage container as a metered volume from either a single dose or multi dose container. If a multi dose container is used, it is preferably equipped with a standard metering pump system as used in commercial nasal spray products. If the liquid storage container is cylindrical, it is preferred that the diaphragm has a circular design and the vibration generator has an annular design. The inhalation nebulizer includes an aerosol generator and a mixing chamber having an inhalation valve and an exhalation valve.

Preferably, the aerosol generator is arranged in a section of the mixing chamber that is also of a cylindrical design. Thereby an annular gap is obtained around the aerosol generator through which the ambient air can flow into the mixing chamber during the inhalation phase.

A mouthpiece is preferably integrally formed with the mixing chamber, but it also can be attached removably to the mixing chamber. A subject inhales the aerosol through the mouthpiece. The aerosol is generated by the aerosol generator and is stored in the mixing chamber. The size and the form of the mouthpiece can be chosen such that it enlarges the mixing chamber and simultaneously provides for the arrangement of the exhalation valve. The exhalation valve is preferably located adjacent the opening of the mouthpiece facing the subject.

When a subject exhales into the opening of the mouthpiece, the exhalation valve is opened so that the respiratory air of the subject is discharged into the surroundings. To this end, a valve element of the exhalation valve is lifted and frees the opening of the exhalation valve. The inhalation valve is closed when the subject exhales into the inhalation nebulizer, as the valve element of the inhalation valve closes the opening of said valve. When a subject inhales through the opening of the mouthpiece, the inhalation valve is opened and frees the opening as the valve element is lifted. Thereby ambient air flows through the inhalation valve and the annular gap into the mixing chamber and is inhaled by the subject together with the aerosol. As aerosol has accumulated in the mixing chamber during an exhalation phase, there is available to the subject an increased amount of aerosol, a so-called aerosol bolus, especially at the beginning of an inhalation phase.

Aerosols of the invention may be dispersed by jet, ultrasonic nebulizer, or electronic nebulizer. Alternatively, the formulation may be administered as a dry powder using a metered dose inhaler or a dry powder inhaler, for example. Aerosolized formulations deliver high concentrations of an agent that increases Nrf2 activity directly to airways with low systemic absorption, and include for example, inhalation solutions, inhalation suspensions, and inhalation sprays. Inhalation solutions and suspensions are aqueous-based formulations containing the agent and, if necessary, additional excipients. Such formulations are intended for delivery to the respiratory airways by inspiration.

One factor to be considered in pulmonary delivery is reaching the deep lung. To achieve high concentrations of an agent of the invention in both the upper and lower respiratory airways, the agent is preferably nebulized in jet nebulizers, an ultrasonic nebulizer, or an electronic nebulizer particularly those modified with the addition of one-way flow valves, such as for example, the Pari LC Plus™ nebulizer, commercially available from Pari Respiratory Equipment, Inc., Richmond, Va., which delivers up to 20% more drug than other unmodified nebulizers.

The pH of the formulation is also important for aerosol delivery. When the aerosol is acidic or basic, it can cause bronchospasm and cough. A comfortable range of pH depends on a patient's tolerance. An aerosol solution having a pH between 5.5 and 7.0 is usually considered tolerable. To avoid bronchospasm, the pH of the formulation is preferably maintained between 5.5 and 7.0, most preferably between 5.5 and 6.5 to permit generation of an agent aerosol well tolerated by patients without any secondary undesirable side effects, such as bronchospasm and cough. Propellants, such as HFA 134a, HFA 227, or combinations thereof, may also be used in the formulation. If desired, excipients that promote drug dispersion or enhance valve lubrication may also be formulated with the agent.

### Dry Powder Formulation

As an alternative therapy to aerosol delivery, agents of the invention are administered in a dry powder formulation for efficacious delivery into the endobronchial space. Such formulations have several advantages, including product and formulation stability, high drug volume delivery per puff, and low susceptibility to microbial growth. Therefore, dry powder inhalation and metered dose inhalation are most practical when high amounts of an agent need to be delivered. Depending on the efficiency of the dry powder delivery device, effective dry powder dosage levels typically fall in the range of about 20 to about 60 mg. The invention therefore provides a sufficiently potent formulation of an agent that increases Nrf2 activity in dry powder or metered dose form of drug particles. Such a formulation is convenient because it does not require any further handling such as diluting the dry powder. Furthermore, it utilizes devices that are sufficiently small, fully portable and tend to have a long shelf life.

Aerosol formulation techniques, which can be applied for use in the present invention, are described, e.g., by Sciarra, “Aerosols,” Chapter 92 in Remington's Pharmaceutical Sciences, 16th edition (ed. A. Osol), pp. 1614-1628. Use of pMDIs has some drawbacks, such as employing chlorofluorocarbon propellants, which are damaging to the environment. Thus, alternatives, such as any powder inhalers, spacer devices, and holding chambers, can be used (see, e.g., Malcolmson et al., PSTT 1(9):394-398, 1998, and Newman et al., “Development of New Inhalers for Aerosol Therapy,” in Proceedings of the Second International Conference on the Pharmaceutical Aerosol, pp. 1-20).

For dry powder formulations of the invention, an agent of the invention is milled to a powder having mass median aerodynamic diameters ranging from 1-15 microns by media milling, jet milling, spray drying, super-critical fluid energy, or particle precipitation techniques.

Particle size determinations may be made using a multi-stage Anderson cascade impactor or other suitable method. Alternatively, the dry powder formulation may be prepared by spray drying or solution precipitation techniques. Spray drying has the advantage of being the least prone to degrading the agent. Solution precipitation is performed by adding a co-solvent that decreases the solubility of a drug to a uniform drug solution. When sufficient co-solvent is added the solubility of the drug falls to the point where solid drug particles are formed which can be collected by filtration or centrifugation. Precipitation has the advantage of being highly reproducible and can be performed under low temperature conditions, which reduce degradation. Super-critical fluid technology can produce particles of pharmaceutical compounds with the controlled size, density and crystallinity ideal for powder formulations.

The dry powder formulations of the present invention may be used directly in metered dose or dry powder inhalers. Currently, metered dose inhaler technology is optimized to deliver masses of 1 microgram to 5 mg of a therapeutic. Spacer technology, such as the aerochamber, may also be utilized to enhance pulmonary exposure and to assist patient coordination.

An alternate route of dry powder delivery is by dry powder inhalers. There are two major designs of dry powder inhalers, device-metering designs in which a reservoir of drug is stored within the device and the patient “loads” a dose of the device into the inhalation chamber, and the inspiratory flow of the patient accelerates the powder out of the device and into the oral cavity. Alternatively, dry powder inhalers may also employ an air source, a gas source, or electrostatics, in order to deliver the agent. Current technology for dry powder inhalers is such that payload limits are around 10 mg of powder. The dry powder formulations are temperature stable and have a physiologically acceptable pH of 4.0-7.5, preferably 6.5 to 7.0.

In other embodiments, an agent that increases Nrf2 activity is administered as a liquid aerosol. In solution, the concentration of the agent is about 0.5, 1, 5, 10, 20, 40, 60, 80, 100 mg/mL, or more and is formulated in a physiologically acceptable solution, preferably in one quarter strength of normal saline. Ideally, the subject is administered with at least 10, 50, 100, 200, 500, 700, 1000, or more than 1000 micrograms of an agent of the invention administered as an aerosol. The use of dry powder inhalation preferably results in the delivery of at least about 1, 5, 10, 20, 30, 40, 50, 60, or more than 60 mg of the agent to the respiratory airways of the patient receiving treatment. Patient inspiration techniques, such as breath holding for example, may also optimize deposition of the agent.

It will be understood, however, that the specific dose level for any particular patient will depend on a variety of factors, including the activity of the specific compound employed; the age, body weight, general health, and sex of the individual being treated; the time and route of administration; the rate of excretion; other drugs that have previously been administered; and the severity of the particular disease undergoing therapy.

In other embodiments, the invention features a device comprising an agent that increases Nrf2 activity, one or more propellants, and if desired, a surfactant. The liquefied propellant serves as an energy source to expel the formulation from the valve in the form of an aerosol, and as a dispersion medium for the drug and surfactant. The surfactant lubricates the metering valve mechanism, and helps disperse drug particles. Drug dissolution usually necessitates the addition of less volatile ethanol. In one embodiment, the device is a metered-dose inhaler (MDI). Typically, an MDI is provided a molded plastic actuator which positions the subject's lips very close to the spray orifice. Optionally, it may also be provided with a spacer, which is a hollow tube that provides for enhanced delivery of the agent.

The formulations can be administered to human patients in therapeutically effective amounts (e.g., amounts which prevent, eliminate, or reduce a pathological condition) to provide therapy for a disease associated with corticosteroid resistance (e.g., COPD, asthma, including severe asthma, acute graft-versus host disease, autoimmune inner ear disease, inflammatory bowel diseases, rheumatoid arthritis), as well as for the treatment of respiratory infections (for example, Rhinovirus, coronovirus, influenza A and B, parainfluenza, Adenovirus, and Respiratory syncytial virus), particularly those associated with chronic obstructive pulmonary disease. The preferred dosage of a nucleobase composition of the invention is likely to depend on such variables as the type and extent of the disorder, the overall health status of the particular patient, the formulation of the compound excipients, and its route of administration.

Generally, doses of compositions that increase Nrf2 biological activity or expression would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Kits

The invention provides kits for preventing, treating, or monitoring a disease associated with corticosteroid resistance in COPD and other corticosteroid-resistant inflammatory diseases, such as asthma, including severe asthma, acute graft-versus host disease, autoimmune inner ear disease, inflammatory bowel diseases, rheumatoid arthritis, as well as bacterial infections, including those associated with COPD and related conditions (e.g. smoking, chronic bronchitis). In another embodiment, the kit comprises an agent (e.g., sulforaphane) that increases Nrf2 expression or biological activity, and directions for the use of the agent in the treatment of a bacterial infection associated with COPD. If desired, the kit further comprises a corticosteroid and/or a KEAP1 inhibitor. In particular embodiments, a kit of the invention provides an agent that increases Nrf2 activity in combination with a corticosteroid (e.g., dexamethasone, flunisolide, fluticasone propionate, triamcinolone acetonide, beclomethasone dipropionate, budesonide, prednisone, prednisolone, and methylprednisolone). In other embodiments, the kit comprises a sterile container that contains the primer, probe, antibody, or other detection regents; such containers can be boxes, ampoules, bottles, vials, tubes, bags, pouches, blister-packs, or other suitable container form known in the art. Such containers can be made of plastic, glass, laminated paper, metal foil, or other materials. The instructions will generally include information about dosages; methods for using the enclosed materials for the treatment or prevention of chronic obstructive pulmonary disease; precautions; warnings; indications; clinical or research studies; and/or references. The instructions may be printed directly on the container (when present), or as a label applied to the container, or as a separate sheet, pamphlet, card, or folder supplied in or with the container.

### Combination Therapies

Compositions and methods of the invention may be used in combination with any conventional therapy known in the art. In one embodiment, an agent that activates Nrf2 is used in combination with a corticosteroid known in the art. Exemplary corticosteroids include, for example, dexamethasone, flunisolide, fluticasone propionate, triamcinolone acetonide, beclomethasone dipropionate, budesonide, prednisone, prednisolone, and methylprednisolone. In one embodiment, the corticosteroid is administered prior to, concurrently with, or following administration of the agent that activates Nrf2 or with the corticosteroid. Such administration may be, for example, within about 1-5 hours, 6-12 hours, 12-24 hours, or within days (e.g., 1-7 days) or even within 3-5 weeks or more.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Sulforaphane Treatment Enhanced Secretion of Secretory Leukocyte Protease Inhibitor (SLPI)

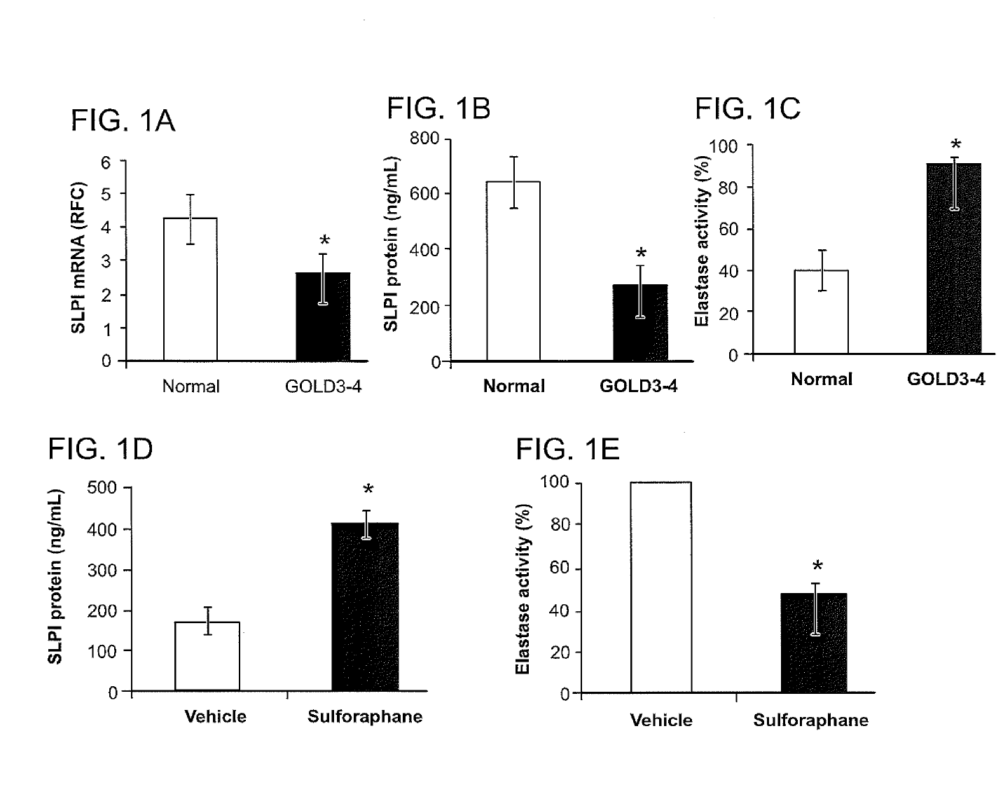

Sulforaphane is a potent pharmacological activator of Nrf2 that increases endogenous antioxidants (A. T. Dinkova-Kostova et al., Proc Natl Acad Sci USA 99, 11908 (Sep. 3, 2002); T. W. Kensler et al., Cancer Epidemiol Biomarkers Prey 14, 2605 (November, 2005); T. A. Shapiro et al., Nutr Cancer 55, 53 (2006); R. K. Thimmulappa et al., Cancer Res 62, 5196 (Sep. 15, 2002)). Neutrophils mediate pathogenesis of COPD by secreting the protease elastase. Neutropil elastase causes proteolysis of extracellular matrix that results in parenchymal destruction and emphysema. A significant decline in SLPI was noted along with greater elastase activity in the lungs of COPD patients. More importantly, sulforaphane treatment was found to enhance SLPI—a potent elastase inhibitor produced by alveolar macrophages. Cell free media from alveolar macrophages treated with sulforaphane showed greater levels of SLPI as well as inhibition of elastase activity (FIGS. 1A-1E). These results indicated that sulforaphane treatment enhanced secretion of the protease inhibitor, secretory leukocyte protease inhibitor (SLPI), a potent neutrophil elastase inhibitor in alveolar macrophages of patients with COPD.

### Example 2

### Inoculation with Viral Ligand Poly I:C Caused Greater Emphysema and Inflammation in Lungs of Mice with a Deletion of Nrf2 in Macrophages and Neutrophils

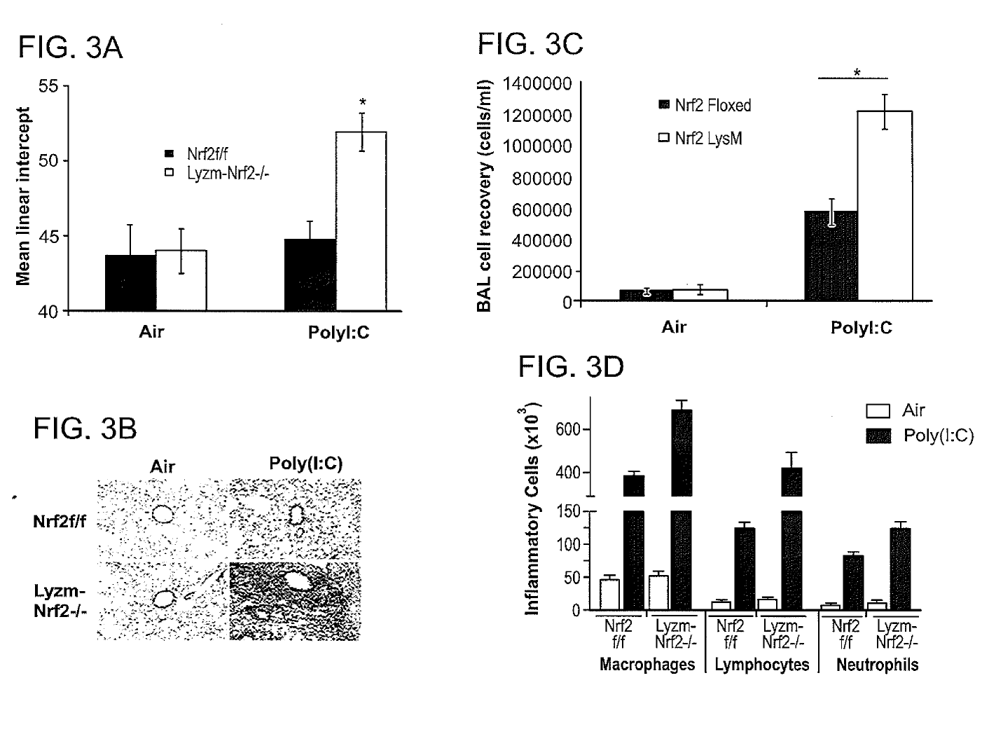

Mice were inoculated with Poly I:C or given control treatments of filtered air. Mean linear intercept was measured as an indication of emphysema (FIG. 3A). Histopathological analysis of the lungs showed greater inflammation in the Lyzm-Nrf2^{-/-} mice (FIG. 3B) and this was confirmed by analysis by bronchoalveolar lavage (BAL) fluid assay (FIG. 3C). Lyzm-Nrf2^{-/-} showed increase levels of macrophages, lymphocytes and neutrophils in response to poly(I:C) (FIG. 3D).

### Example 3

### Activation of Nrf2 by Sulforaphane Enhanced Bacterial Recognition, Phagocytosis and Clearance by Alveolar Macrophages from Patients with COPD

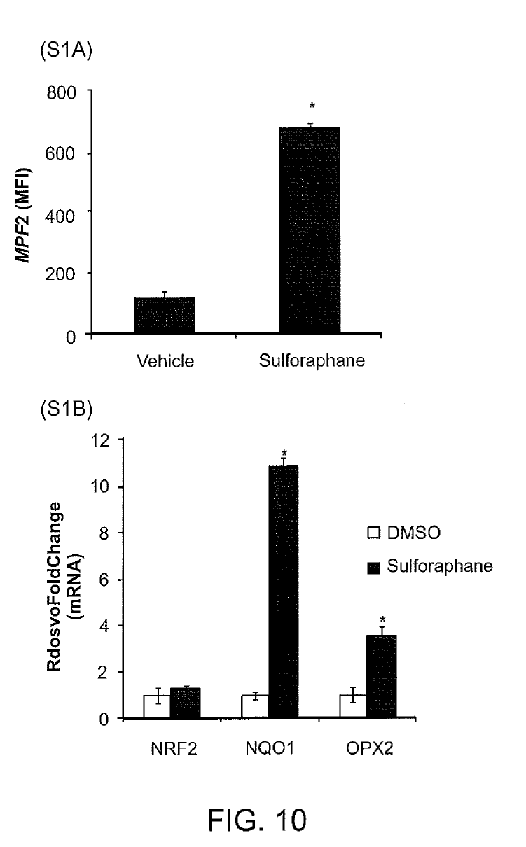

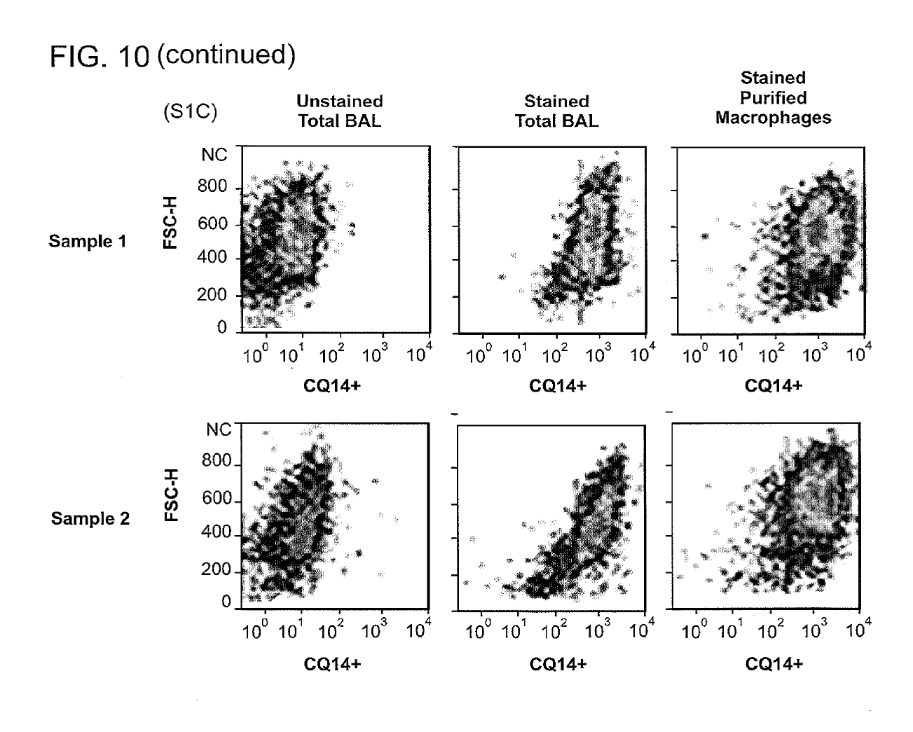

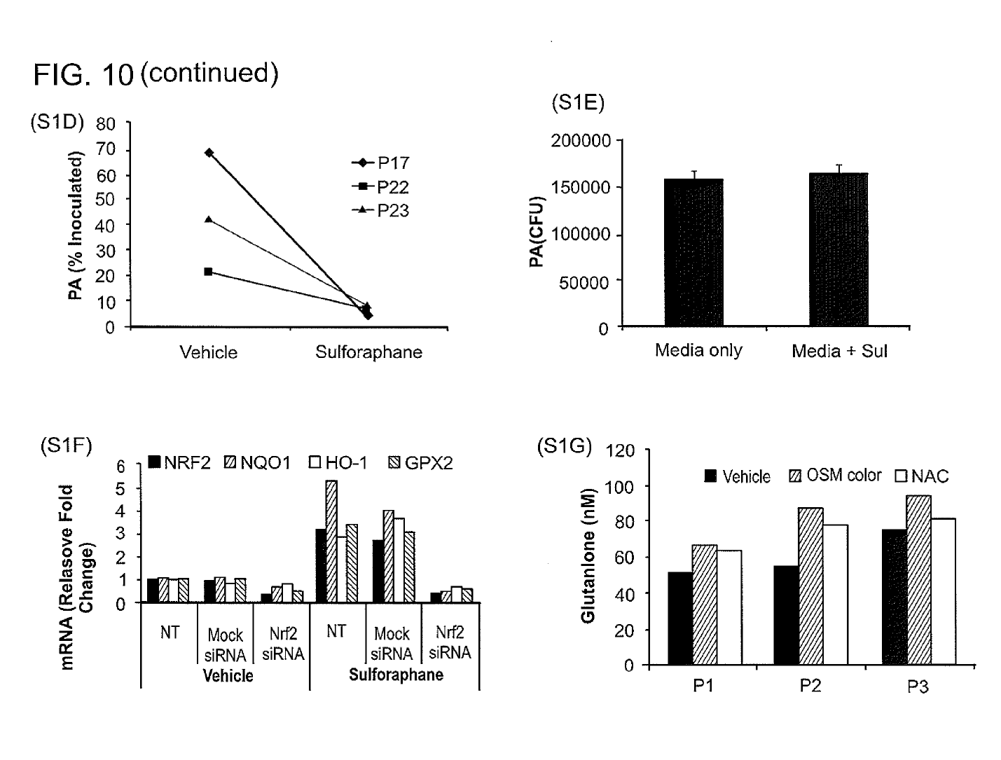

Sulforaphane was found to enhance Nrf2 activity in alveolar macrophages from patients with COPD. Sulforaphane treatment resulted in elevated levels of Nrf2 protein as measured by flow cytometry (FIG. 10A) and subsequent upregulation of Nrf2-regulated targets genes (NQO1 & GPX2) (FIG. 10B).

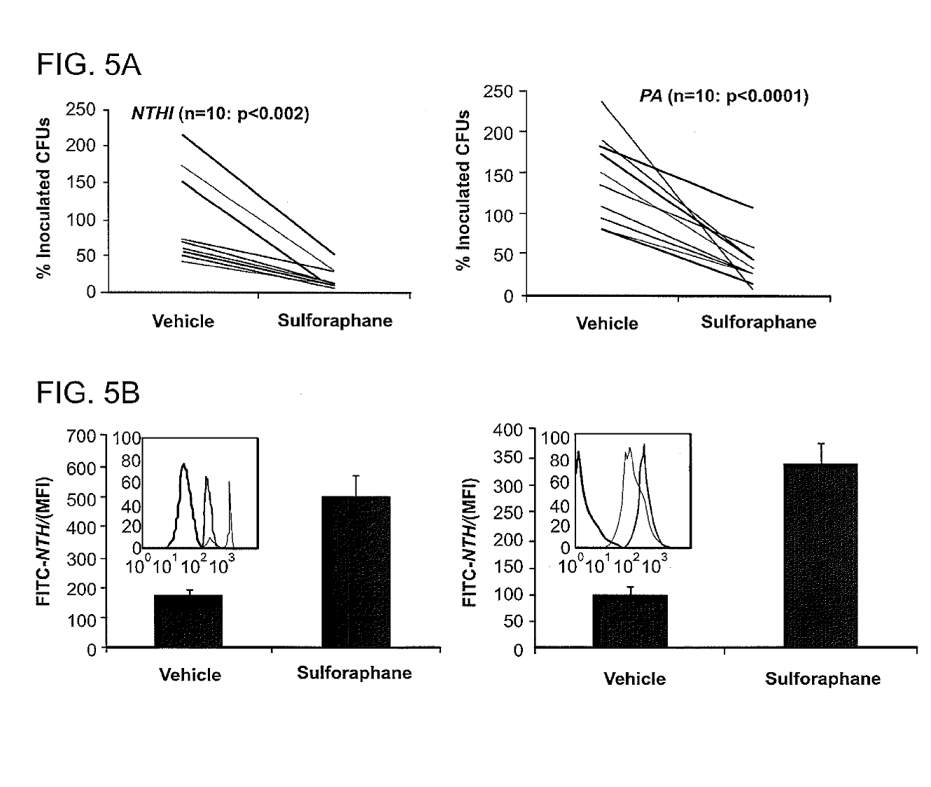

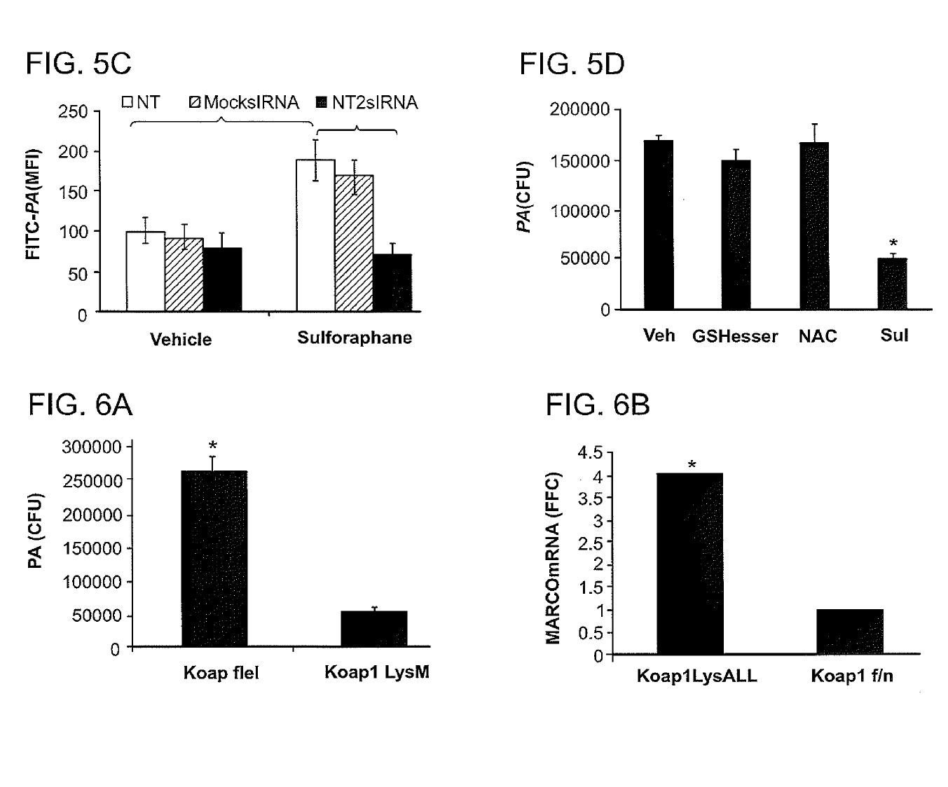

To determine whether sulforaphane enhances bacterial phagocytosis, alveolar macrophages were collected by bronchoalveolar lavage from patients with COPD. The macrophages were treated with sulforaphane treatment and sixteen hours later the macrophages were exposed to isolates of either Pseudomonas aeruginosa or nontypeable Haemophilus influenzae. The purity of isolated macrophages was routinely >95% as measured by a proprietary Romanowski stain, termed Diff-quick staining, and confirmed by flow cytometry for a few initial clinical samples (FIG. 10C). Significant differences in the baseline clearance of Pseudomonas aeruginosa or nontypeable Haemophilus influenzae by alveolar macrophages from individual patients with COPD were observed. However, sulforaphane treatment markedly enhanced clearance (40-95%) of Pseudomonas aeruginosa or nontypeable Haemophilus influenzae by alveolar macrophages obtained from all patients with COPD compared to vehicle, as evident from the decrease in colony forming units (CFU) obtained from culture medium, regardless of smoking status (FIG. 5A). Of note, it was also observed that sulforaphane treatment significantly enhanced bacterial clearance by alveolar macrophages from non-COPD subjects (FIG. 10D). Cell-free medium with sulforaphane alone showed no bactericidal activity towards Pseudomonas aeruginosa or nontypeable Haemophilus influenzae indicating that sulforaphane does not directly cause bacterial killing (FIG. 10E). Next, whether the decrease in bacterial colonization in the culture medium of alveolar macrophages after sulforaphane treatment was due to increased bacterial phagocytosis was investigated. To assess phagocytic ability, alveolar macrophages were incubated with attenuated FITC-labeled Pseudomonas aeruginosa or nontypeable Haemophilus influenzae, and phagocytosed bacteria were analyzed by flow cytometry. Sulforaphane significantly enhanced phagocytosis of Pseudomonas aeruginosa or nontypeable Haemophilus influenzae by alveolar macrophages compared to vehicle (FIG. 5B).

To determine if the effect of sulforaphane on bacterial phagocytosis was mediated by Nrf2, alveolar macrophages were transfected with Nrf2-siRNA and or control siRNA. Nrf2 siRNA transfection caused ˜70-80% knockdown of Nrf2 gene and repressed induction of Nrf2-regulated antioxidant genes following sulforaphane treatment when compared to control siRNA (FIG. 10F). Sulforaphane treatment enhanced bacterial phagocytosis of alveolar macrophages transfected with control ssRNA; however, failed to do so in Nrf2-siRNA transfected macrophages (FIG. 5C). To examine whether the improvement in bacterial clearance by alveolar macrophages after sulforaphane treatment was dependent on Nrf2-regulated antioxidant function, alveolar macrophages were treated with N-acetyl cysteine (NAC) or GSH-ethyl ester (GSH-e). NAC or GSH-e increased intracellular glutathione levels (FIG. 10G) but failed to improve bacterial clearance (FIG. 5D) by alveolar macrophages. Taken together, the data indicate that sulforaphane improves bacterial phagocytosis and clearance by COPD alveolar macrophages by activating Nrf2 which is independent of upregulation of antioxidants.

### Example 4

### Nrf2 Mediates Transcriptional Regulation of the Scavenger Receptor MARCO

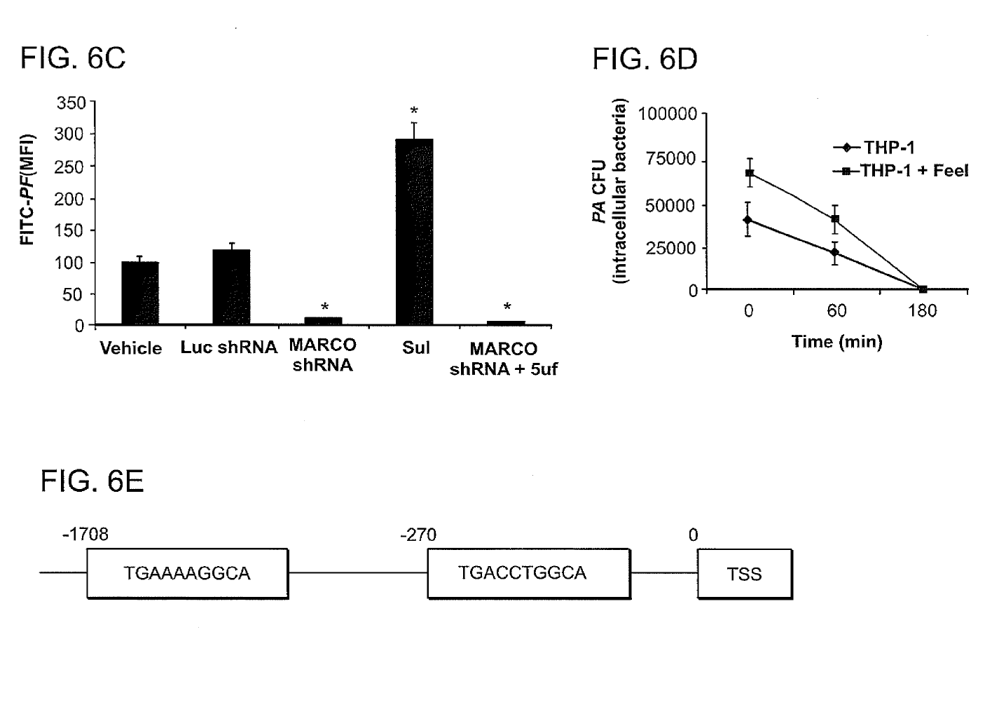

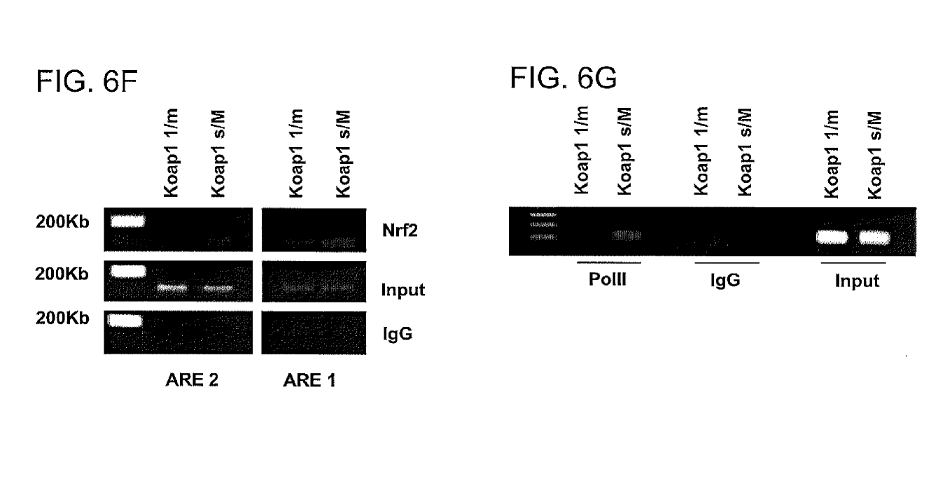

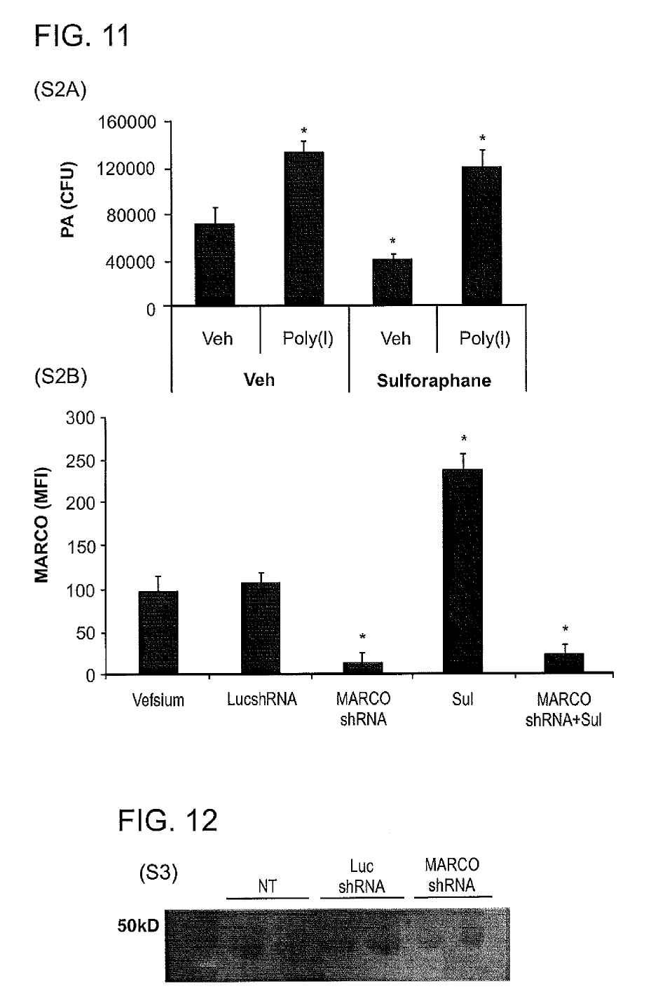

The above data collectively indicate that the activation of Nrf2 improves bacterial phagocytosis and clearance by COPD macrophages. The dependence of Nrf2 on bacterial phagocytosis was investigated. Alveolar macrophages with high endogenous Nrf2 activity, isolated from conditional knockout mice with deletion of the Nrf2 inhibitor Keap1 specifically in myeloid cells (Lyzm-Keap1^{-/-} mice) showed increased bacterial phagocytosis and clearance when compared to Keap1 flox/flox (Keap1^{f/f}) mice (FIG. 6A). This observations confirmed Nrf2-dependent modulation of bacterial phagocytosis and clearance. To determine the mechanisms by which Nrf2 regulates phagocytosis, microarray analysis was employed. Microarray analysis and quantitative (q) RT-PCR validation revealed higher basal gene expression of MARCO in BM-DM of Lyzm-Keap1^{-/-} mice compared to Keap1^{f/f} mice (FIG. 6B). To address whether sulforaphane-induced expression of MARCO correlates with bacterial clearance, differentiated human THP-1 macrophages were used. Bacterial clearance by sulforaphane treated THP-1 macrophages after exposure with polyinosinic acid (Poly(I)) was analyzed. Poly(I) is a non-selective class A scavenger receptor blocker that inhibits MARCO mediated bacterial uptake and clearance (G. M. DeLoid, T. H. Sulahian, A. Imrich, L. Kobzik, PLoS One 4, e6209 (2009)). Poly(I) significantly impaired sulforaphane ability to improve bacterial clearance by THP-1 macrophages (FIG. 11A). Next, phagocytosis of FITC-Pseudomonas aeruginosa was examined in THP-1 macrophages with knockdown of MARCO using lentivirus-encoded shRNA (MARCO shRNA).

Endogenous levels of MARCO (FIG. 11B) and phagocytic activity (FITC-PA) were both suppressed in THP-1 macrophages infected with MARCO shRNA when compared to control luciferase (Luc)-shRNA (FIG. 6C). Sulforaphane treatment significantly elevated phagocytosis of FITC-Pseudomonas aeruginosa in THP-1 macrophages transfected with Luc-shRNA, but not in MARCO-shRNA (FIG. 6C). Together, these findings indicate that Nrf2 regulates bacterial phagocytosis by increasing MARCO in macrophages. In addition, down-regulation of MARCO by genetic manipulation is associated with a decrease in bacterial phagocytosis.

Since reactive oxygen species formation is important for bactericidal activity in macrophages, whether up-regulation of Nrf2-dependent antioxidants by sulforaphane interferes with the bacterial killing after the bacteria has been internalized by the THP-1 macrophages was determined. The colony forming units obtained from macrophages as a function of time is reflective of intracellular killing ability. The efficiency of intracellular Pseudomonas aeruginosa killing was found to be similar between vehicle and sulforaphane treatment, although there was a moderate increase in the kinetics of intracellular killing after sulforaphane treatment. These results rule out any detrimental effect from increased Nrf2 regulated antioxidants after sulforaphane treatment in the phagocytosis process (FIG. 6D).

Next, to examine whether the MARCO gene is a transcriptional target of Nrf2, the mouse genomic sequence in the 5′ UTR of MARCO was analyzed. Genomics software analysis revealed the presence of two AREs: ARE1 and ARE2, which were located −270 bp and −1708 bp upstream of the transcription start site (TSS), respectively, (FIG. 6E). CHIP assay showed greater Nrf2 binding to ARE1 and ARE2 as well as binding of RNA pol II in the promoter of MARCO gene in macrophages isolated from Lyzm-Keap1^{-/-} mice compared to Keap1^{f/f} mice indicating active gene transcription in the former group (FIGS. 6F & 6G). Taken together, these results suggest that Nrf2 mediates transcriptional regulation of MARCO.

### Example 5

### Sulforaphane Increases Bacterial Phagocytosis by Increasing MARCO in COPD Alveolar Macrophages

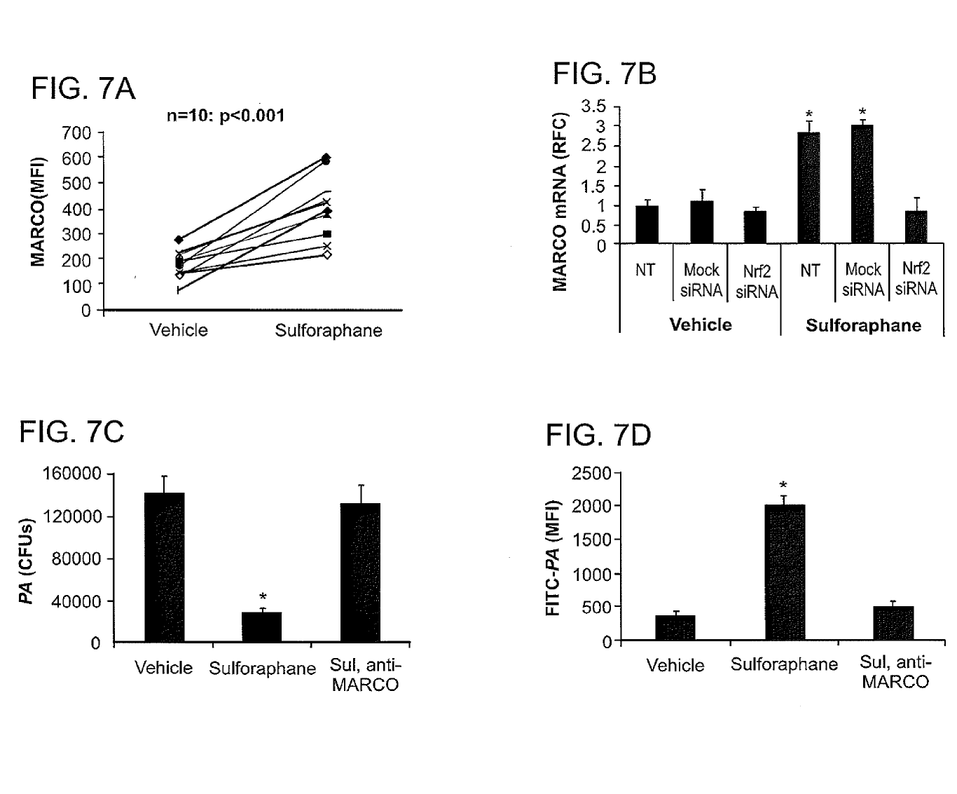

Next, whether sulforaphane increased the expression of MARCO in alveolar macrophages from patients with COPD was investigated. Compared to vehicle treatment, sulforaphane treatment significantly elevated surface expression of MARCO protein, measured by flow cytometry, in alveolar macrophages from patients with COPD (2- to 5-fold, p<0.001) compared to vehicle (FIG. 7A). However, sulforaphane failed to upregulate the expression of MARCO mRNA in alveolar macrophages transfected with Nrf2-siRNA when compared to control ssRNA, indicating Nrf2-dependent regulation of MARCO (FIG. 7B). To examine the contribution of MARCO in mediating bacterial phagocytosis by alveolar macrophages following sulforaphane treatment, an antibody-depletion approach was employed. The specificity of anti-MARCO antibody was validated by immunoblot analysis (FIG. 12). Incubation of alveolar macrophages post-sulforaphane treatment with anti-MARCO antibody abrogated bacterial clearance (FIG. 7C) and phagocytosis of attenuated FITC-labeled Pseudomonas aeruginosa (FIG. 7D). Taken together, sulforaphane restored bacterial phagocytosis and improved clearance in alveolar macrophages by Nrf2-dependent upregulation of MARCO. To assess the effect of sulforaphane on the phenotype of macrophages, CD80/86, MHC-11, CD14 and CD206 were analyzed by FACS. Moderate suppression of receptors CD80/86 and CD206 was noted, but no significant difference in the expression of other markers was seen.

### Example 6

### Cigarette Smoke Exposure Impaired Alveolar Macrophage Phagocytosis and Enhanced Bacterial Colonization in the Lungs

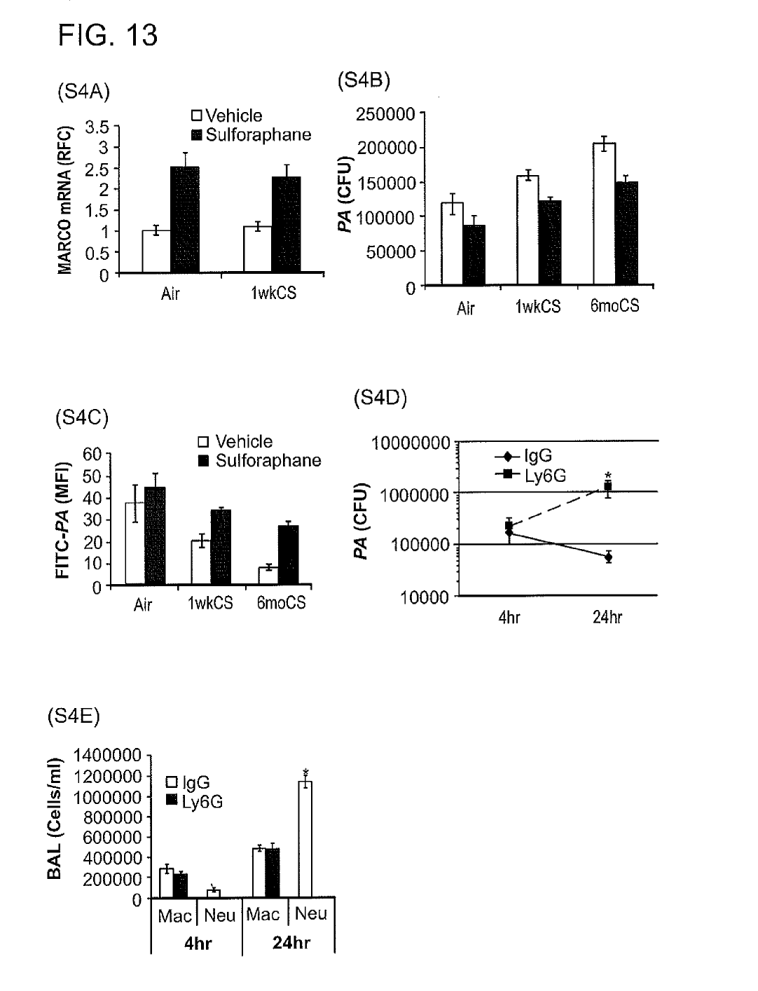

Cigarette smoke exposure for 6 months causes emphysema in mouse models (T. Rangasamy et al., J Clin Invest 114, 1248 (November, 2004); T. E. Sussan et al., Proc Natl Acad Sci USA, (Dec. 22, 2008)). To address, in vivo role of Nrf2 on pulmonary bacterial clearance, mouse models exposed to cigarette smoke were used. Six months (long-term) as well as one week (short term) cigarette smoke exposure mouse models were used. Alveolar macrophages from mice exposed to cigarette smoke for 6 months showed significant impairment in bacterial (Pseudomonas aeruginosa) uptake and clearance (FIG. 11A-B) compared to macrophages isolated from mice exposed to filtered-air. Of note, even short-term 1 week of exposure to cigarette smoke induced significant defect in alveolar macrophages phagocytic ability (FIGS. 7A and 11B). Further, bacterial incubation increased MARCO gene expression in alveolar macrophages from filtered-air but not in CS-exposed mice (FIG. 13A). However, sulforaphane treatment increased MARCO expression in alveolar macrophages (FIG. 13A) from mice exposed-CS and increased bacterial phagocytic ability and clearance (FIGS. 13B and 13C).

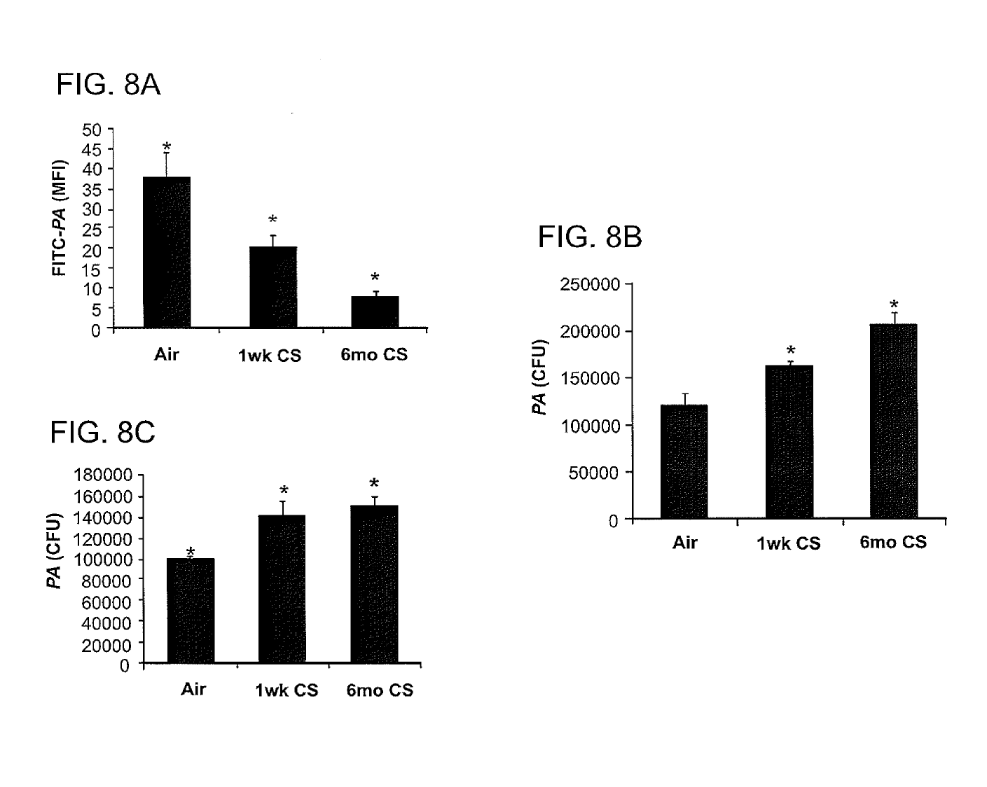

To assess the in vivo role of alveolar macrophages in bacterial clearance following acute respiratory infection, the role of macrophage—versus neutrophil-mediated bacterial clearance was investigated. Neutrophil depletion by Ly6G—specific antibody administration showed no effect on bacterial clearance at 4 h; however, significantly increased bacterial colonization in lungs at 24 hours of Pseudomonas aeruginosa infection (FIG. 13D). After PA infection, predominantly macrophages were found until 4 hours, however later neutrophils infiltrate and become the predominant cell type by 24 hours in brochoalveolar lavage fluid (FIG. 13E). Based on these findings, to assess the role of alveolar macrophages in pulmonary bacterial clearance, bacterial colonization at 4 hours after bacterial infection for future studies were analyzed. Mice exposed to 1 week or 6 months of CS exposure showed greater pulmonary bacterial colonization compared to filter air (FIG. 8C).

### Example 7

### Sulforaphane Reduced Bacterial Colonization and Inflammation in the Lungs of Mice after Cigarette Smoke Exposure

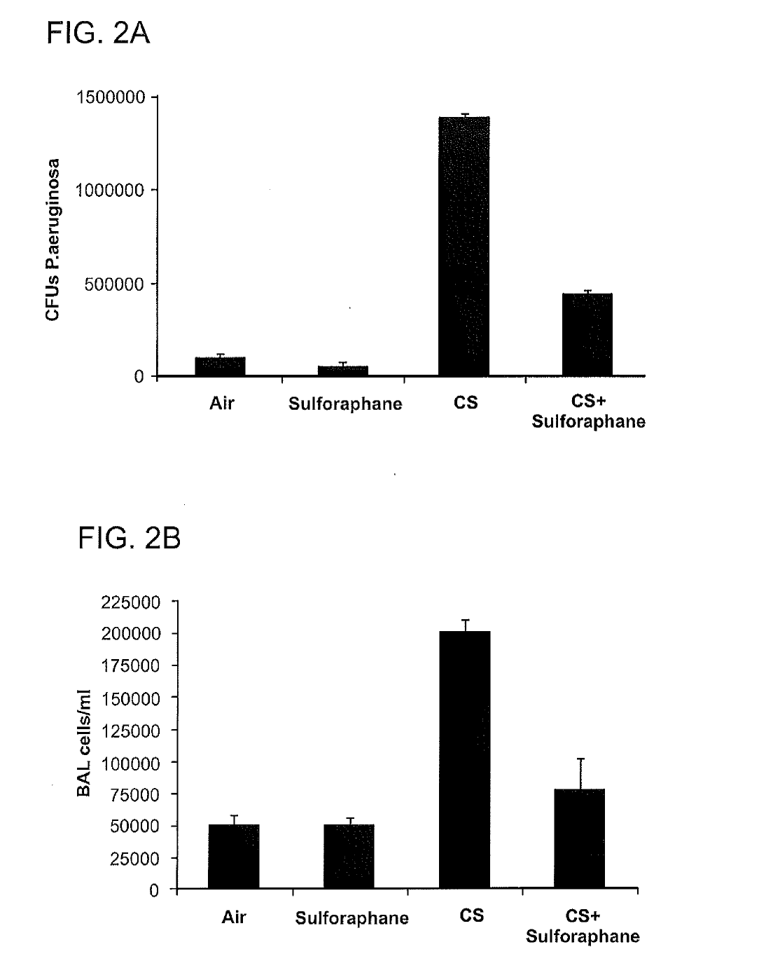

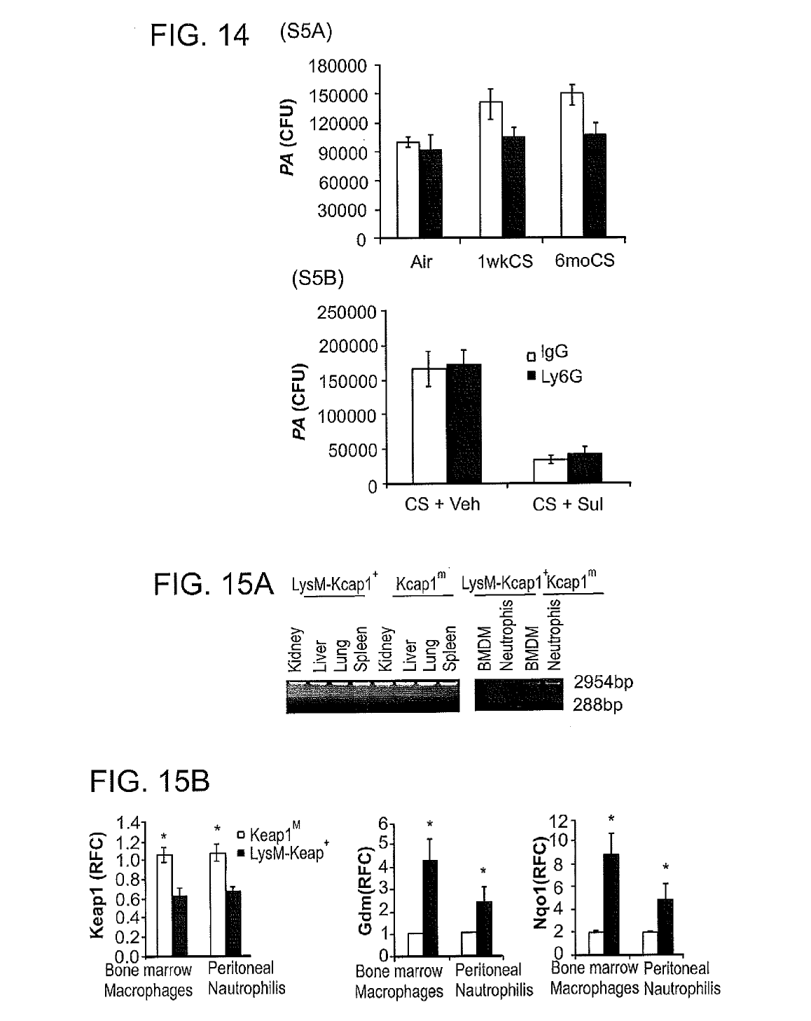

The efficacy of sulforaphane to improve bacterial phagocytosis and clearance in the lungs of mice exposed to cigarette smoke was determined. After 6 months or 1 week of CS or air exposure, mice were treated with sulforaphane and or vehicle (0.5 mg/mouse/day) for three consecutive days using an Aeroneb® nebulizer (Aerogen, Inc., Galway, Ireland). Mice were challenged intranasally with Pseudomonas aeruginosa 24 hours after the last dose of sulforaphane, and bacterial burden in BALF was analyzed 4 hours later. Sulforaphane treatment significantly reduced bacterial burden in lungs of mice exposed to 1 week (FIG. 2A) or 6 months (FIG. 14A) compared to vehicle. A significant impairment in lung bacterial clearance even 3 days after cigarette smoke exposure was observed. A similar decrease in bacterial burden was observed after sulforaphane treatment in neutrophil-depleted cigarette smoke-exposed mice (FIG. 14B). Concurrent with bacterial burden, sulforaphane treatment significantly reduced inflammation in the lungs of mice (FIG. 2B). These results demonstrate the efficacy of sulforaphane in vivo to inhibit bacterial colonization by improving bacterial phagocytosis and inflammation in an Nrf2-dependent manner.

### Example 8

### Dietary Consumption of Sulforaphane-Enriched Broccoli Sprout Extract (BSE) Increased MARCO Expression

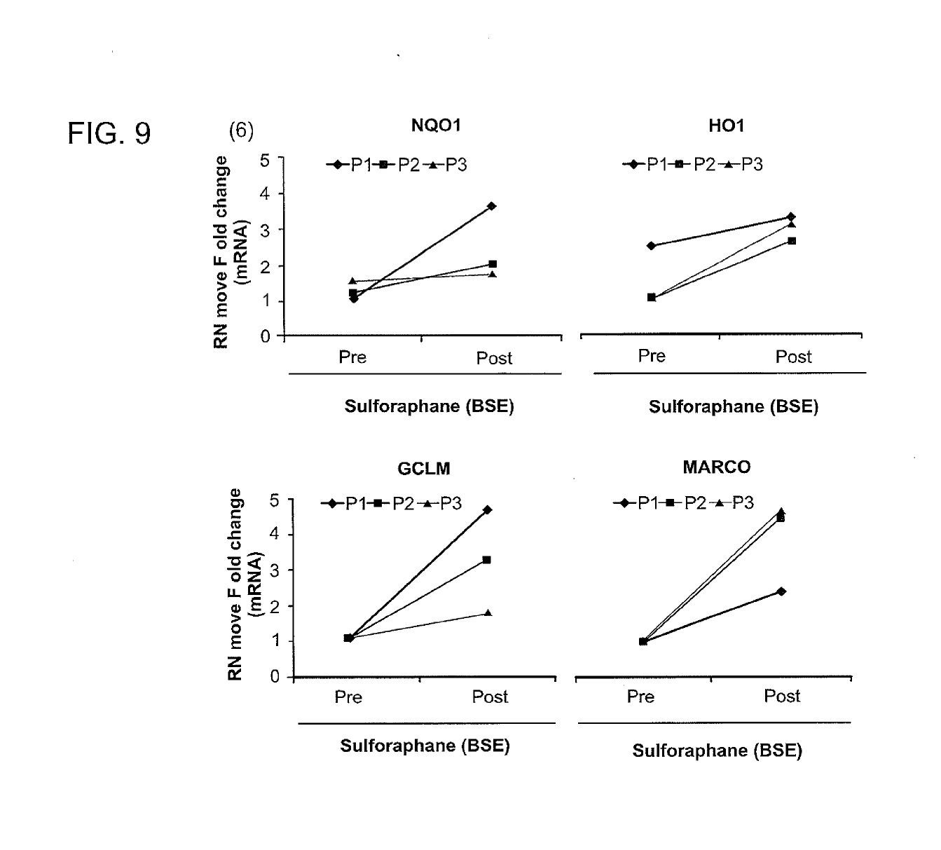

Sulforaphane-enriched broccoli sprout extract has been evaluated in human subjects for anti-cancer properties (T. W. Kensler et al., Cancer Epidemiol Biomarkers Prey 14, 2605 (November, 2005); J. W. Fahey, Y. Zhang, P. Talalay, Proc Natl Acad Sci USA 94, 10367 (Sep. 16, 1997)) and shown to increase Nrf2-regulated antioxidants in upper airways (M. A. Riedl, A. Saxon, D. Diaz-Sanchez, Clin Immunol 130, 244 (March, 2009)). Whether sulforaphane increases the expression of MARCO in human subjects was determined. Peripheral blood monocytes (PBMC) were isolated from three healthy subjects before and after they consumed broccoli sprout extract for two weeks. Sulforaphane significantly increased the expression of MARCO (2-5 fold) and other Nrf2-regulated antioxidant (NQO1, GCLM and HO-1) genes in PBMC of healthy subjects (FIG. 9). These results indicate that sulforaphane treatment is likely to increase MARCO in humans, just as it does in mice.

### Example 9

### Sulforaphane Improved Corticosteroid Responsiveness in Alveolar Macrophages

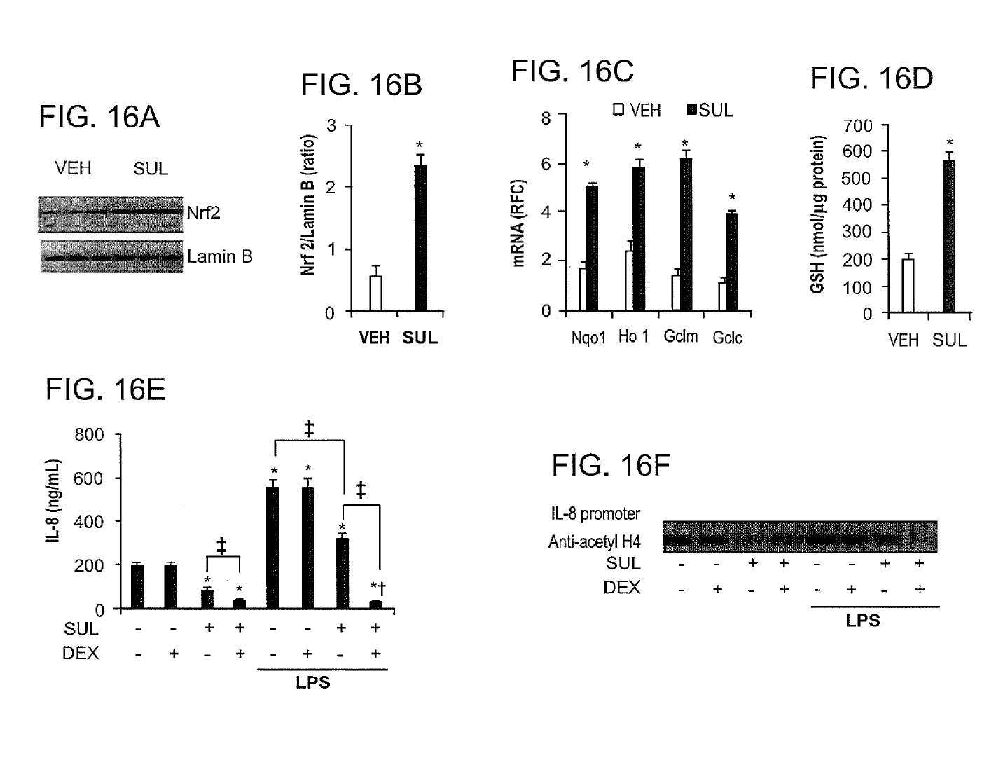

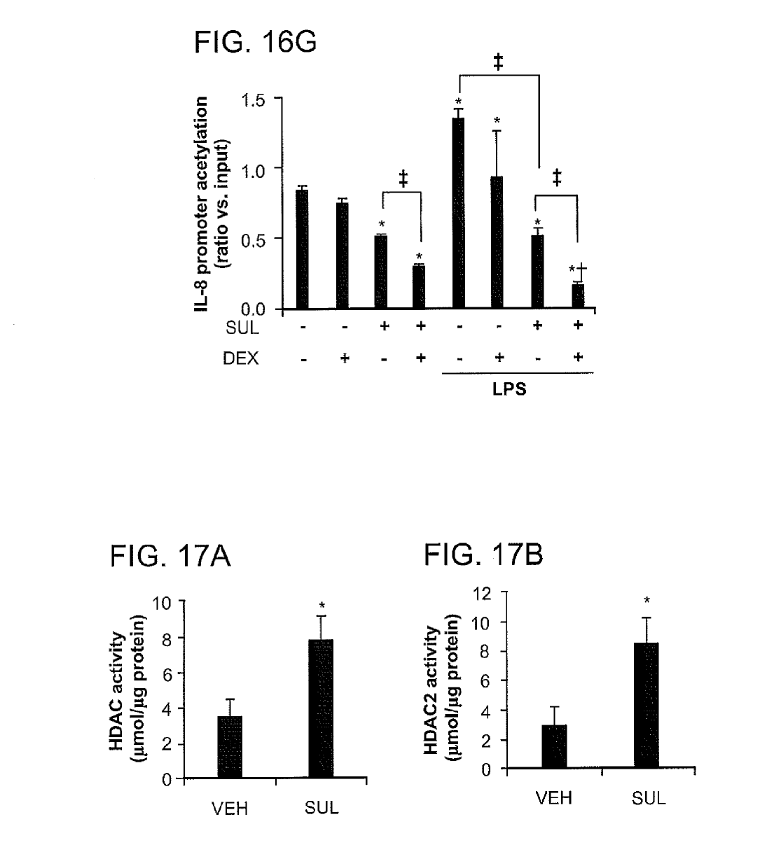

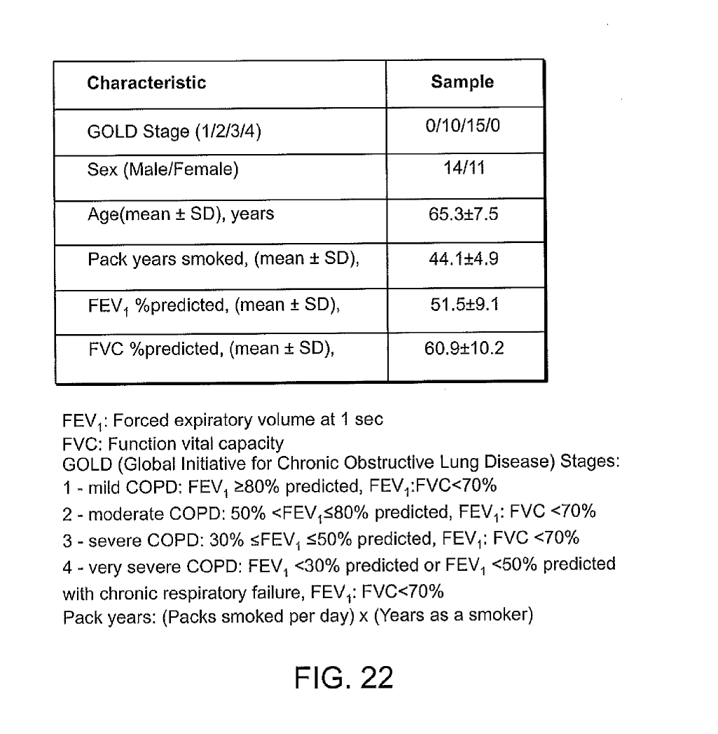

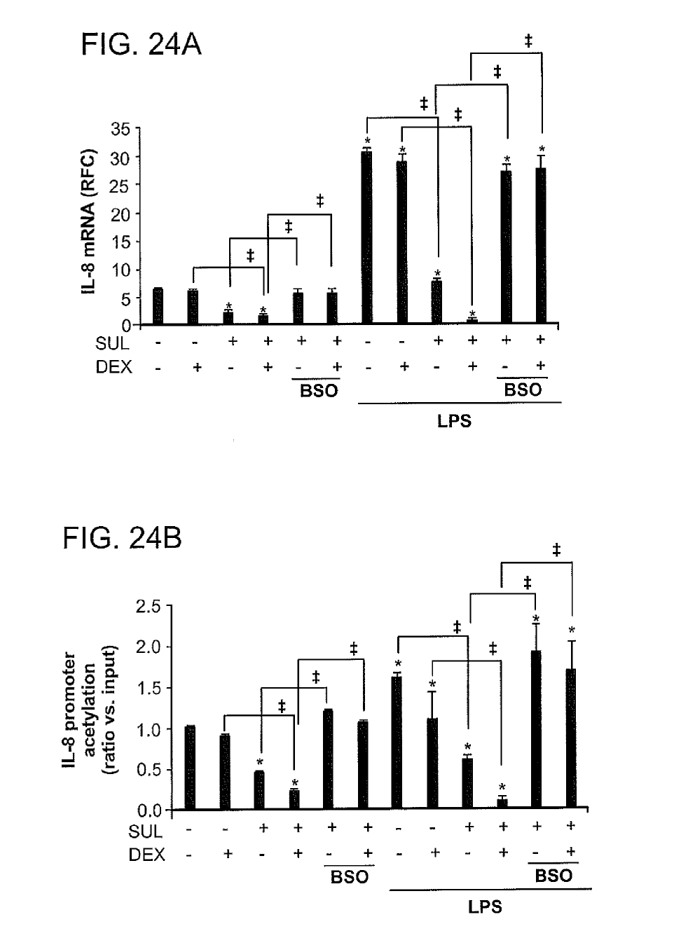

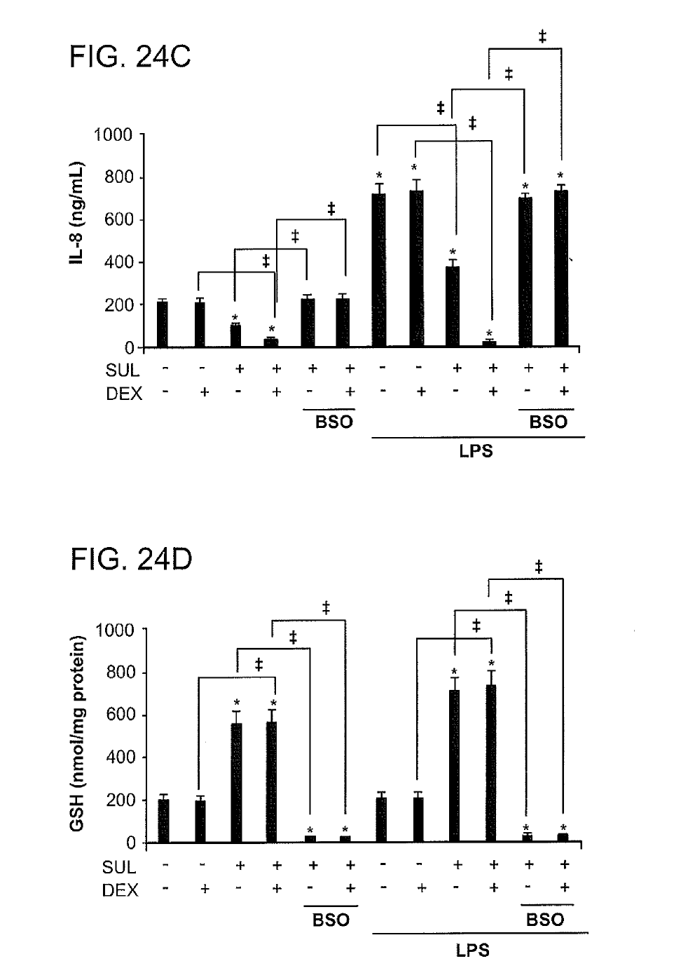

Detailed characteristics of patients used for isolation of alveolar macrophages and peripheral lung tissues are provided in FIG. 22. Treatment with sulforaphane significantly elevated Nrf2 nuclear protein levels, Nrf2-regulated antioxidant genes (NQO1, HO-1), GSH biosynthesizing enzymes (GCLC, GCLM), and GSH levels in alveolar macrophages (FIGS. 16A-D). The ability of dexamethasone (DEX), a synthetic corticosteroid, to suppress lipopolysaccharide (LPS)-induced cytokine expression after sulforaphane treatment was analyzed. Consistent with results from previous studies (Ito, K., et al., J Exp Med 203, 7-13 (2006)), dexamethasone alone failed to suppress LPS-induced IL-8 secretion (FIG. 16E). Sulforaphane treatment significantly restored DEX's inhibitory effect on basal and LPS-induced IL-8 secretion (5 fold reduction; P<0.01; N=25 patients) with a concomitant reduction in the levels of histone acetylation on the IL-8 promoter and expression of the IL-8 gene (FIG. 16E-16G; FIG. 24A). Up-regulation of glutathione synthesis is critical for the ability of sulforphane to restore corticosteroid sensitivity because sulforaphane failed to improve DEX sensitivity in alveolar macrophages as evident from failure to reduce histone acetylation on the IL-8 gene promoter or inhibit IL-8 expression in the presence of buthionine sulfoximine (BSO), a specific inhibitor of glutathione synthesis (FIG. 24A-24D). Sulforaphane alone significantly reduced IL-8 levels basally and after LPS treatment.

### Example 10

### Sulforaphane Improves Corticosteroid Sensitivity by Increasing HDAC2 Activity by Targeting Nrf2

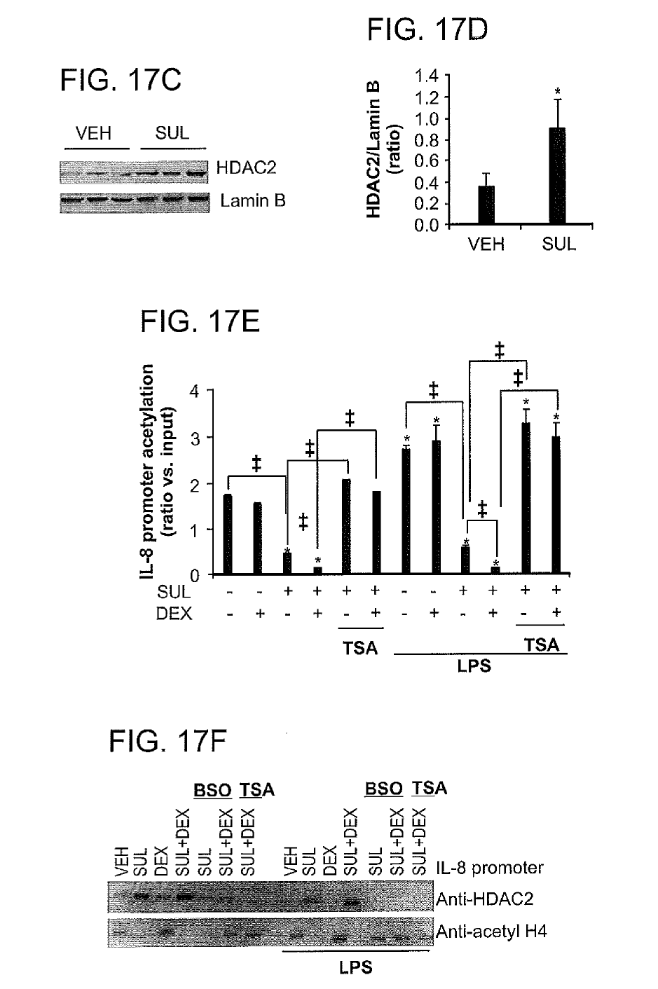

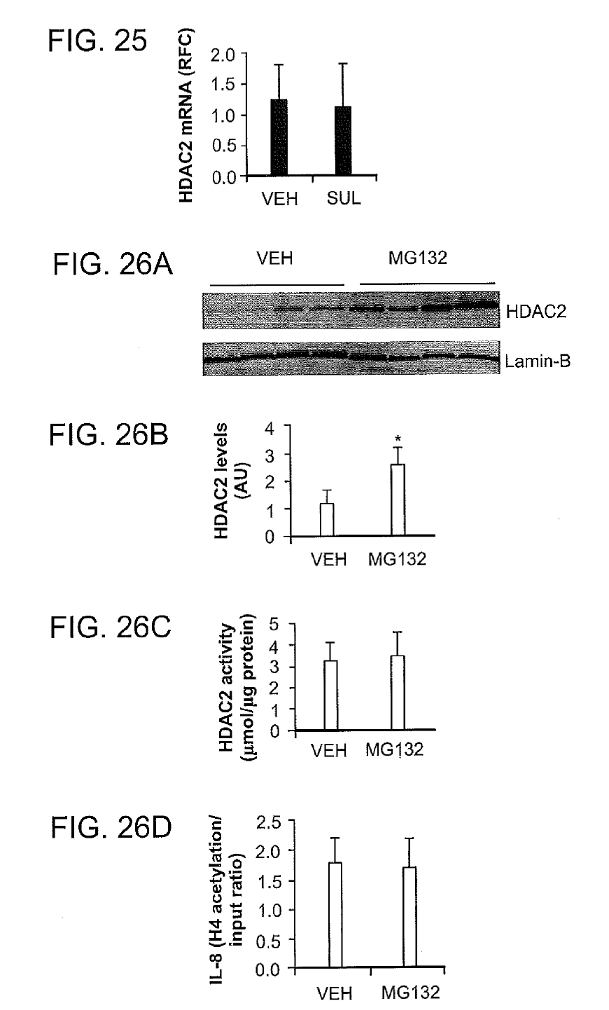

Whether sulforaphane modulated histone deacetylase (HDAC2) activity was determined. Sulforaphane significantly elevated the enzymatic activity of total HDAC (FIG. 17A) and HDAC2 (approximately 2 fold; P<0.01 versus vehicle, FIG. 17B) and up-regulated HDAC2 protein levels in alveolar macrophages (FIGS. 17C and 17D) without altering the levels of HDAC2 mRNA (FIG. 25).

Acetylation of histone H4 in the IL-8 promoter correlates with expression of IL-8 gene in lungs of patients with COPD (Ito, K., et al., N Engl J Med 352, 1967-1976 (2005)). Next, the levels of acetylated histone H4 associated with the IL-8 promoter in the presence of the HDAC inhibitor trichostatin (TSA) was determined. Cotreatment with TSA ablated sulforaphane repressed basal and LPS-induced histone acetylation in the IL-8 promoter region with and without DEX. This indicates that sulforaphane restores corticosteroid sensitivity in alveolar macrophages by increasing HDAC activity (FIG. 17F). Sulforaphane also significantly increased the levels of HDAC2 binding in the IL-8 promoter that was further enhanced after DEX treatment at baseline and after LPS treatment. Concomitantly, a decrease in histone acetylation in IL-8 promoter was found (FIG. 17F). In the presence of buthionine sulfoximine, sulforaphane failed to increase HDAC2 binding in the IL-8 promoter indicating the dependence on Nrf2-dependent GSH synthesis (FIG. 17F).

### Example 11

### Sulforaphane Decreased Posttranslational Modification of HDAC2 in Alveolar Macrophages

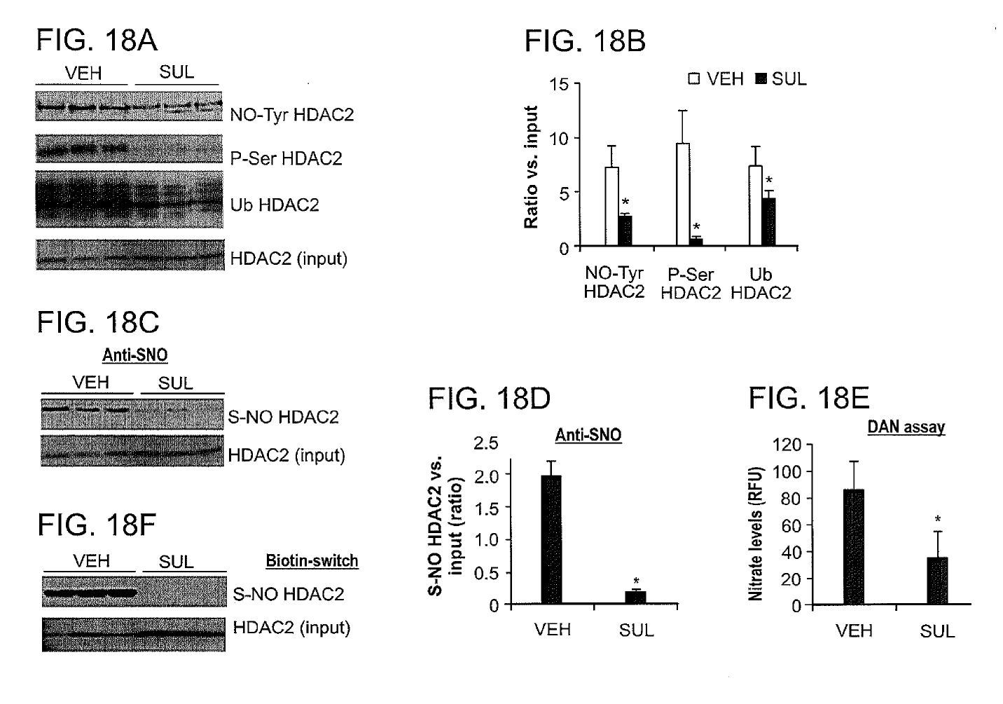

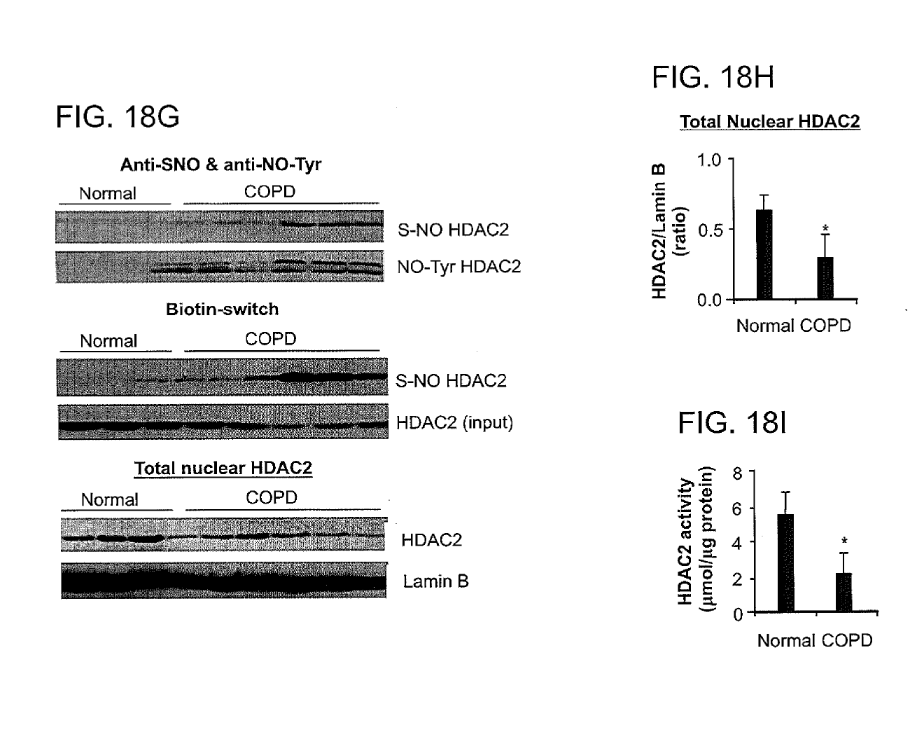

HDAC2 undergoes posttranslational modifications (Adenuga, D., et al., Am J Respir Cell Mol Biol 40, 464-473 (2009); Osoata, G. O., et al., Chest (2009)) after exposure to cigarette smoke, H₂O₂, and NO donor that promotes proteasome-dependent degradation. Although, an increase in total HDAC2 protein was found (FIGS. 26A and 26B), there was no increase in HDAC2 enzymatic activity or HDAC2 binding to the promoter of the IL-8 gene in the presence of the proteasomal inhibitor MG132 in alveolar macrophages (FIGS. 26C and 26D) suggesting that post-translational modification impairs HDAC2 function. It was hypothesized that sulforaphane improved HDAC2 activity by reducing certain posttranslational modifications capable of restoring HDAC's activity. Significant reductions in the levels of tyrosine nitration, serine phosphorylation, and ubiquitination in nuclear immunopurified HDAC2 from alveolar macrophages treated with sulforaphane compared with vehicle were found (FIGS. 18A and 18B). These results suggest that sulforaphane increased HDAC2 activity, in part, by increasing HDAC2 protein stability by decreasing posttranslational modification.

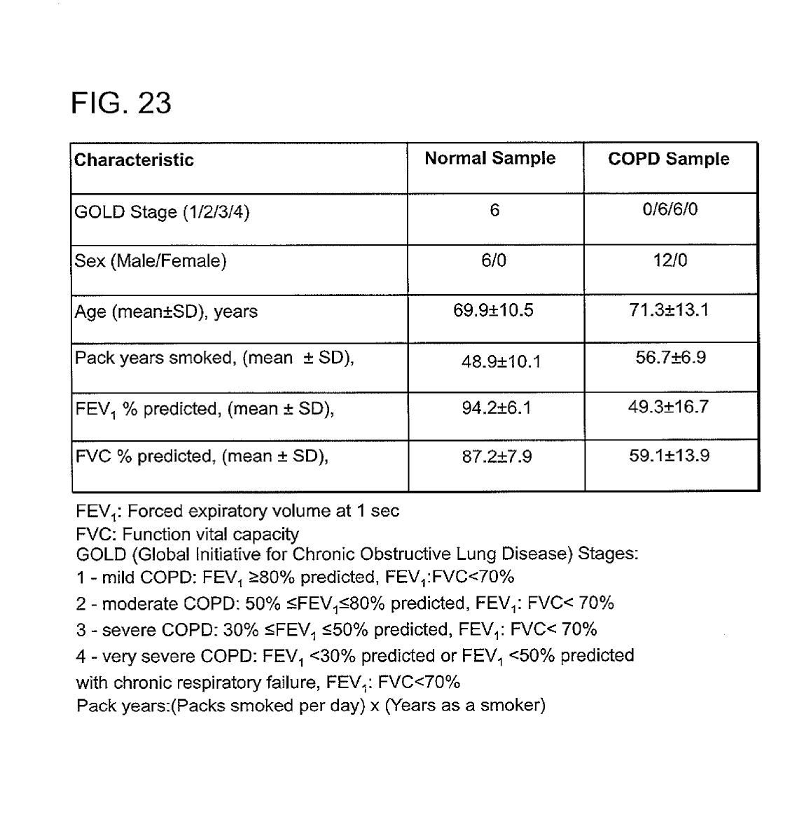

Using anti-S-nitrosylation (SNO) antibody, S-nitrosylation of HDAC2 in alveolar macrophages was significantly reduced after sulforaphane treatment was found (FIGS. 18C and 18D). Further, the 2,3-diaminonaphthalene (DAN) and biotin-switch assays (Jaffrey, S. R. & Snyder, S. H., Sci STKE 2001, p11 (2001); Uehara, T., et al., Nature 441, 513-517 (2006)) were used. The 2,3-diaminonaphthalene and biotin-switch assays also revealed that S-nitrosylation of HDAC2 in alveolar macrophages was reduced after sulforaphane treatment (FIGS. 18E and 18F). Next, to investigate whether S-nitrosylation of HDAC2 correlates with disease severity, peripheral lung tissue isolated from non-COPD smokers and COPD smokers were used (FIG. 23). A significant increase in S-nitrosylation (FIG. 18G) and tyrosine nitration (FIG. 18G) of HDAC2 concurrent with a decrease in HDAC2 protein (FIGS. 18G and 18H) and activity (FIG. 181) in peripheral lung tissues with COPD was found.

### Example 12

### Sulforaphane Restored Corticosteroid Sensitivity by Increasing HDAC2 Activity Via Denitrosylation in Alveolar Macrophages in an Nrf2-Dependent Manner

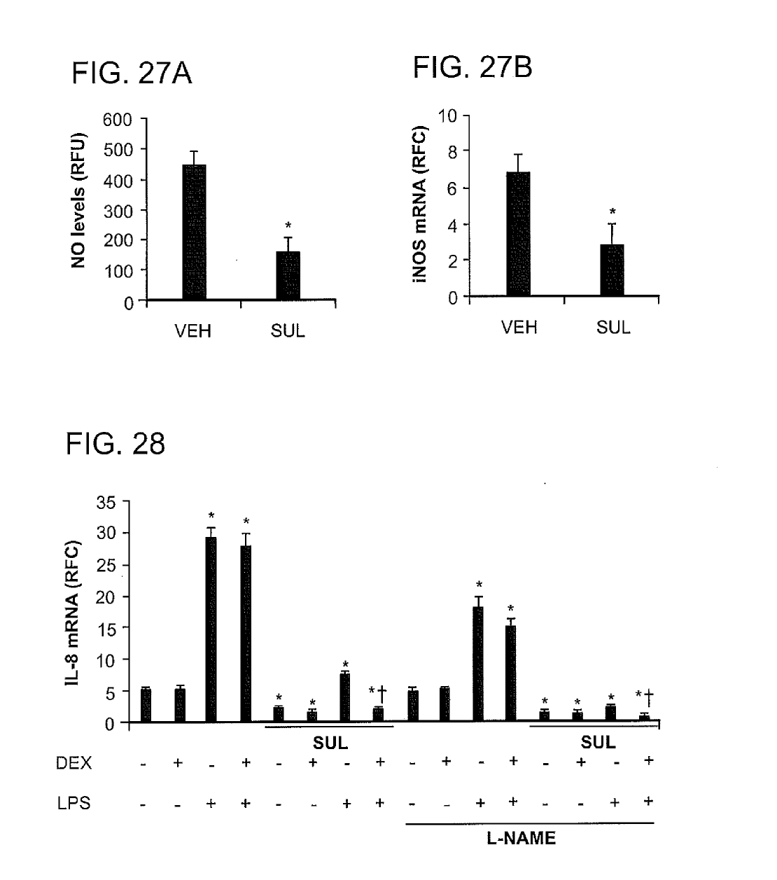

Whether sulforaphane inhibited NO levels in alveolar macrophages was investigated. Alveolar macrophages treated with sulforaphane showed significantly lower levels of NO and iNOS expression than vehicle (FIGS. 27A and 27B).

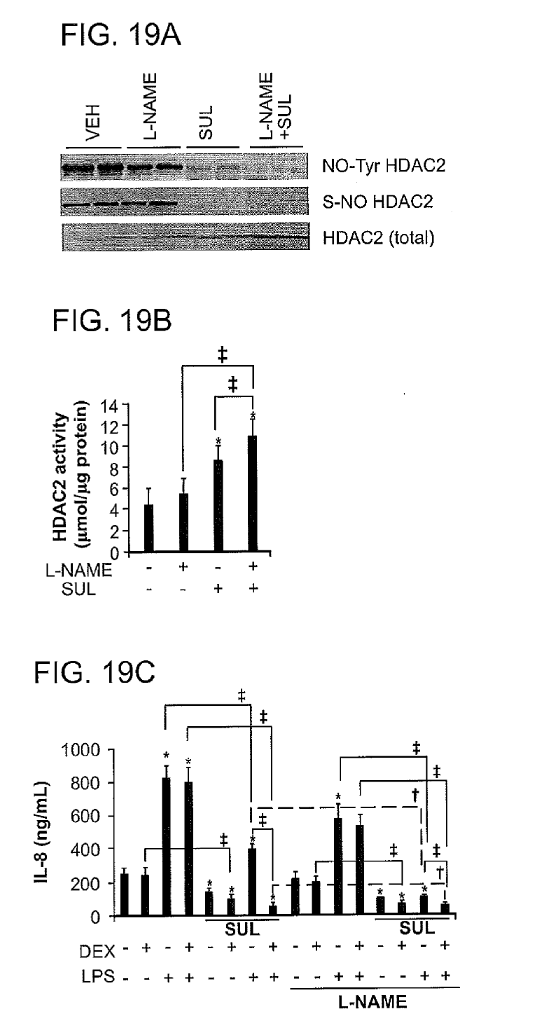

Whether the mechanism of protection by sulforaphane was by means of NO inhibition or a different mechanism was determined. Alveolar macrophages were co-incubated with the NOS inhibitor L-NAME with or without sulforaphane. L-NAME alone significantly decreased levels of nitrotyrosine-HDAC2 but not SNO-HDAC2 (FIG. 19A) and failed to increase HDAC2 enzymatic activity (FIG. 19B) and improve DEX's repressive effect on basal and LPS-induced IL-8 expression (protein, FIG. 19C; mRNA, FIG. 28). However, co-incubation of L-NAME and sulforaphane markedly reduced nitrotyrosine-HDAC2 and SNO-HDAC2 and improved HDAC2 activity concurrent with DEX responsiveness in alveolar macrophages (FIG. 19A-19C) compared with L-NAME or sulforaphane alone. L-NAME or sulforaphane suppressed NO generation, but the reduction was greater after L-NAME treatment. These results indicate that inhibition of NO alone is not sufficient to restore HDAC2 activity and DEX responsiveness in alveolar macrophages. Sulforaphane improved HDAC2 activity and DEX responsiveness in alveolar macrophages by means of a mechanism independent of NO inhibition.

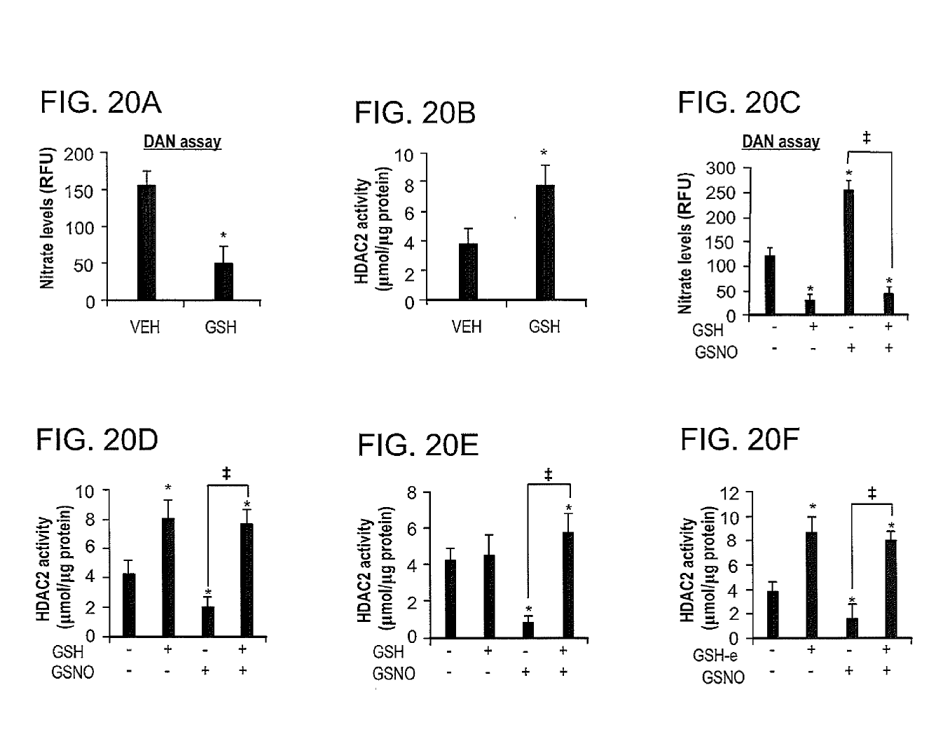

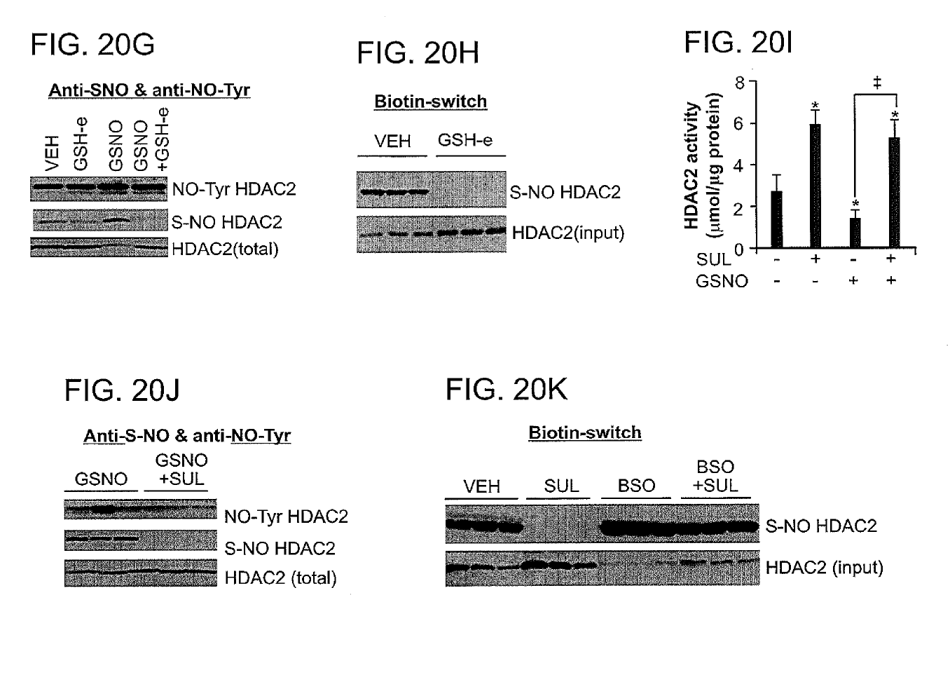

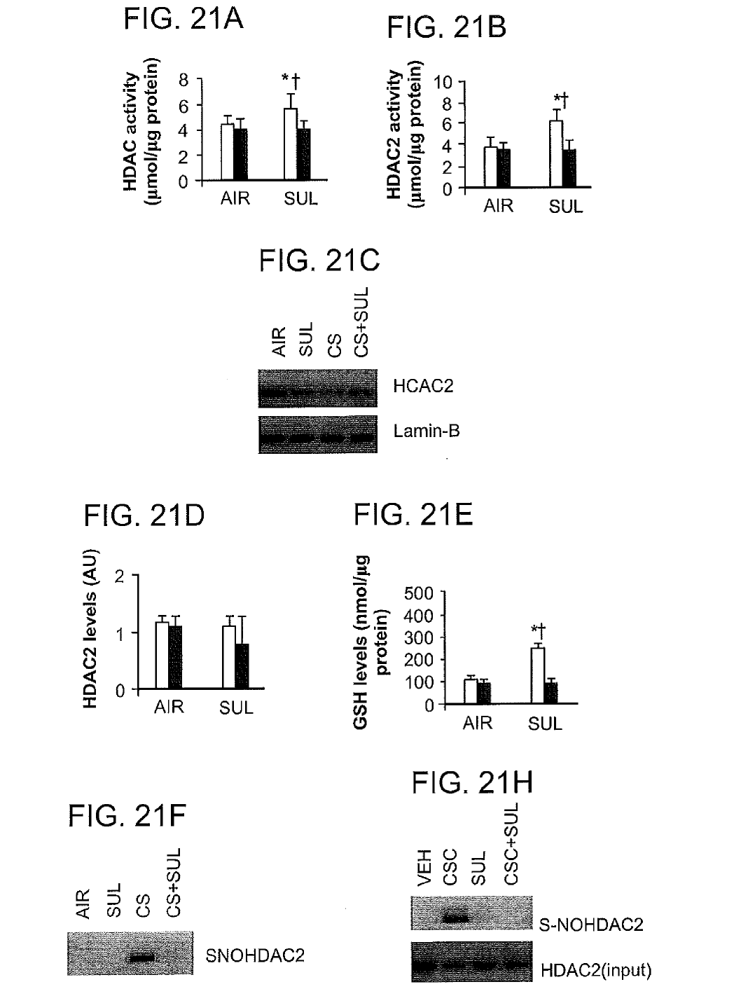

It is unclear from these findings whether sulforaphane restored HDAC2 activity by reducing nitrosative, nitrosylation, or both modifications of HDAC2 protein. Recent evidence indicates that glutathione (GSH) mediates denitrosylation of proteins via a transnitrosylation reaction (Benhar, M. et al., Nat Rev Mol Cell Biol 10, 721-732 (2009)). Because sulforaphane significantly elevated GSH levels and failed to increase HDAC2 activity in the presence of BSO, it was hypothesized that sulforaphane mediates denitrosylation of HDAC2, thereby augmenting HDAC2 activity in alveolar macrophages. Whether GSH mediates denitrosylation of HDAC2 and denitrosylation improves HDAC2 enzymatic activity was investigated. In a series of cell-free in vitro experiments, it was found that (i) denitrosylation by GSH as monitored by using the DAN assay was associated with a concurrent increase in HDAC2 activity (FIGS. 20A and 20B); (ii) to S-nitrosoglutathione (GSNO) (an NO donor) elevated S-nitrosylation and decreased enzymatic activity, which was reversed by GSH treatment (FIGS. 20C and 20D); and (iii) recombinant HDAC2 (rHDAC2) with GSNO significantly inhibited rHDAC2 enzymatic activity that was restored by GSH (FIG. 20E). These results indicate that GSH increases the activity of HDAC2 by denitrosylation in cell-free system.

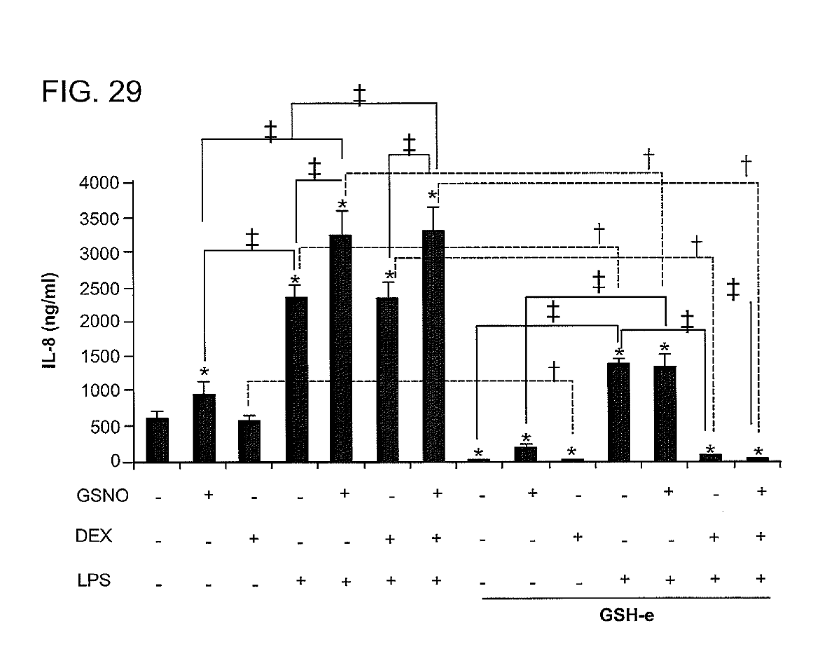

Next, the denitrosylation of HDAC2 after GSH-e or sulforaphane treatment in alveolar macrophages exposed to GSNO was measured. Exposure of alveolar macrophages to GSNO decreased HDAC2 enzymatic activity. However, GSH-e after GSNO treatment significantly elevated HDAC2 enzymatic activity in AMs, and that activity level was equivalent to the level observed with vehicle-treated alveolar macrophages exposed to GSH-e (FIG. 20F). Immunoblot analysis showed significant reduction in the levels of SNO-HDAC2 but not NO-tyr HDAC2 in GSNO-exposed alveolar macrophages after GSH-e treatment (FIGS. 20G and 20H). Similar to GSH-e, sulforaphane exposure post-GSNO treatment significantly increased HDAC2 activity to the level observed with vehicle-treated alveolar macrophages exposed to sulforaphane (FIG. 201). However, unlike GSH-e, sulforaphane decreased the levels of both NO-Tyr HDAC2 and SNO-HDAC2 in GSNO-exposed alveolar macrophages (FIG. 20J). Sulforaphane's ability to reduce the levels of SNOHDAC2 protein was ablated in alveolar macrophages in the presence of buthionine sulfoximine (FIG. 20K). It was observed that N-acetylcysteine (NAC) failed to ameliorate SNO-HDAC2 levels in GSNO-treated AMs. DEX responsiveness in GSNO-treated alveolar macrophages was analyzed. GSNO treatment moderately augmented LPS-induced IL-8 secretion in alveolar macrophages (FIG. 29). GSNO exposure did not further increase DEX resistance in alveolar macrophages. GSH-e treatment significantly increased the repressive effect of DEX on basal and LPS-induced IL-8 secretion in alveolar macrophages. Taken together, these results indicate that sulforaphane via denitrosylation in a GSH-dependent manner restores HDAC2 activity in alveolar macrophages of patients with COPD.

### Example 13

### Denitrosylation of HDAC2 Increases Deacetylation of Glucocorticoid Receptor

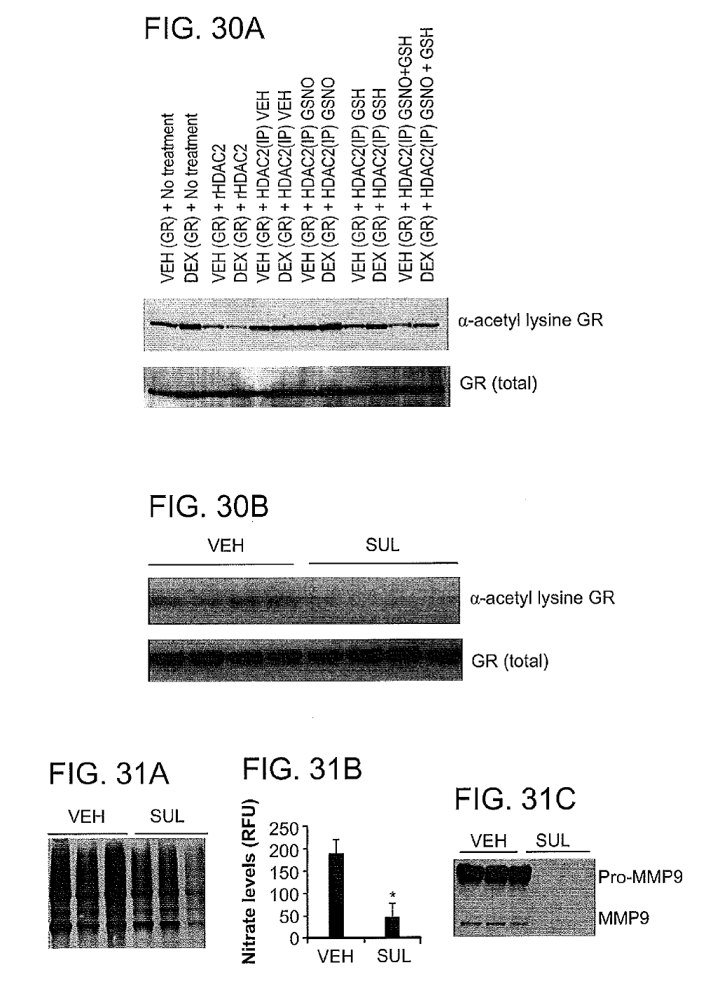

Glucocorticoid receptor is a substrate of HDAC2, and deacetylation of glucocorticoid receptor is required for binding with NF-κB for its anti-inflammatory action. Nuclear glucocorticoid receptor from alveolar macrophages treated with and without DEX was immunopurified and analyzed the ability of immunopurified-HDAC2 from alveolar macrophages to deacetylate glucocorticoid receptor. Alveolar macrophages treated with DEX showed higher levels of acetylated nuclear glucocorticoid receptor than did alveolar macrophages treated with vehicle. HDAC2 or GSNO-exposed HDAC2 failed to mediate deacetylation of glucocorticoid receptor; however, GSH exposure significantly enhanced the deacetylation reaction (FIG. 30A), indicating a restoration of HDAC2 function. In agreement, a significant decrease in the levels of acetylated-glucocorticoid receptor was found in alveolar macrophages treated with sulforaphane compared to vehicle control (FIG. 30B).

### Example 14

### Sulforaphane Decreases Global S-Nitrosylation in AMs

Whether sulforaphane treatment reduced global SNO proteins in alveolar macrophages was tested. Results of immunoblot analysis with anti-SNO antibody showed a significant reduction in SNO proteins in lysates of alveolar macrophages treated with sulforaphane than did alveolar macrophages treated with vehicle; these results were also verified by using the DAN assay (FIGS. 31A and 31B). Using a biotin-switch assay, the S-nitrosylation of the specific protein MMP9 was assessed. S-nitrosylation activates MMP9 (Gu, Z., et al., Science 297, 1186-1190 (2002)), which is thought to be involved in COPD pathogenesis. Sulforaphane treatment significantly reduced the levels of S-nitrosylated MMP9 and secretory levels of active MMP9 (FIG. 31C). These data demonstrate the ability of sulforaphane to mediate denitrosylation of other proteins and suggests that it might have beneficial effects in COPD other than restoration of steroid sensitivity.

### Example 15

### Sulforaphane Restores HDAC2 Activity in Cigarette Smoke-Exposed Lungs of Mice

Whether sulforaphane increase HDAC2 activity in lungs of cigarette smoke exposed mice was determined. It was observed that a significant increase in total HDAC (FIG. 21A) and HDAC2 enzymatic activity (FIG. 21B) and protein (FIGS. 21C and 21D) in the lungs of cigarette smoke-exposed mice after sulforaphane treatment. Sulforaphane treatment increased GSH levels (FIG. 21E) and reduced the levels of SNO-HDAC2 in CS-exposed mice lungs (FIG. 21F). Of note, a significant reduction in the levels of SNO-HDAC2 in CSC-exposed PMs after sulforaphane treatment was also observed (FIG. 21H). Taken together, these results indicate that sulforaphane restores HDAC2 enzymatic activity via GSH-dependent denitrosylation.

The following methods and materials were used in Examples 1 through 8.

### Recruitment of Subjects and Patient Characteristics:

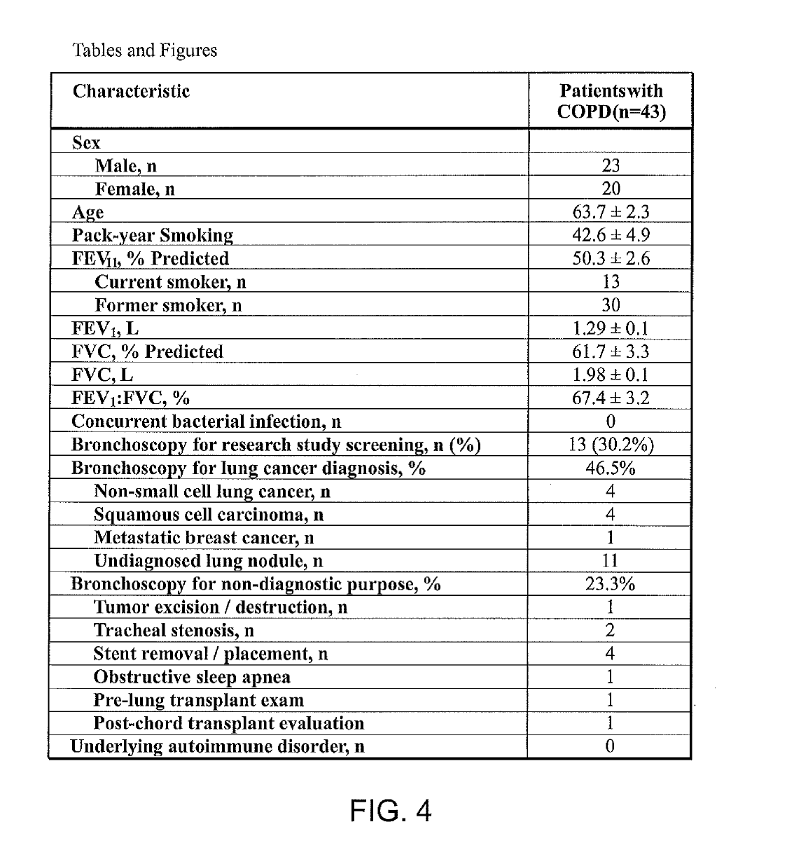

This study was approved by the institutional review board of Johns Hopkins University. Patients with COPD were recruited from clinical populations at Johns Hopkins Hospital and Johns Hopkins Bayview Medical Center. Patients were asked to volunteer for a bronchoscopy for collection of alveolar macrophages by bronchoalveolar lavage (BAL). BAL samples were also collected from patients who underwent bronchoscopy procedures for clinical indications. Informed consent was obtained from all patients. Inclusion criteria included 1) Adult patients with COPD: patients who were already scheduled to undergo bronchoscopy for a clinical indication. These patients had an FEV₁/FVC<0.70, and a FEV₁ 40-80% predicted; and a >10 pack-year smoking history (current and former smokers were included). Exclusion criteria included 1) pregnancy, 2) hemodynamic instability, 3) baseline hypoxemia (SpO2<90% on up to 2 L/min via nasal cannula), and 4) bronchoscopy being performed ‘urgently’ or ‘emergently’ for therapeutic purposes. Demographic data are presented in FIG. 4.

The Broccoli Sprout Extract (BSE) study was approved by the institutional review board of Johns Hopkins University to test whether sulforaphane protects from airway hyper-reactivity in asthma. Volunteers, both healthy and individuals with asthma were recruited from the general population. Subjects were screened using a questionnaire, allergy skin testing, and routine methacholine (Mch) inhalation challenge. Venous blood was drawn from subjects prior to and immediately after they consumed BSE containing 100 μMol of sulforaphane for 14 days. PBMCs isolated from healthy subjects were used.

Forty-three patients with moderate COPD were recruited. Detailed patient characteristics are provided in FIG. 4. Study participants had no concurrent lung infections or prior diagnosis for any underlying autoimmune disorders.

### Cell Culture:

Human alveolar macrophages were purified from individual BAL samples by centrifugation and seeded onto 6-well tissue culture plates. After incubation for 2 h, non-adherent cells were removed and the adherent cells were incubated with R, S-sulforaphane (5 μM; LKT Laboratories, Inc., St. Paul, Minn.) and or DMSO in RPMI 1640 culture medium with 10% FBS and 1% penicillin-streptomycin (Invitrogen, Carlsbad, Calif.). After 16-20 h, adherent cells were scraped, suspended in RPMI 1640 culture media and plated onto 96-well tissue culture plates for bacterial phagocytosis and clearance assays. Cells obtained were >95% macrophages as determined by the morphologic appearance of histologically stained preparations (Diff-Quick). Percent viability of alveolar macrophages by trypan blue exclusion was >90%. For MARCO antibody-depletion, alveolar macrophages were incubated with human anti-MARCO antibody (1 μg/ml; (Hycult, Uden, The Netherlands) for 4 hours prior to bacterial challenge.

Murine alveolar macrophages and bone marrow (BM)-derived macrophages were isolated from Keap1^{f/f} (D. J. Blake et al., Am J Respir Cell Mol Biol, (Jun. 11, 2009)), Lysm-Keap1^{-/-} mice (specific deletion of Keap1 in myeloid cells) was created by crossing with mice bearing Cre recombinase under the control of lysozyme M promoter as described in the supplemental material (FIG. 15). BMDM were isolated by culturing bone marrow cells in the presence of 10 ng/ml granulocyte-macrophage colony-stimulating factor (PeproTech) for 7 days. Murine macrophages were cultured in RPMI 1640 medium supplemented with 10% FBS and 1% penicillin-streptomycin. The human THP-1 cell line (American Type Culture Collection, Manassas, Va.) was grown in RPMI 1640 medium supplemented with 10% non-heat-inactivated fetal bovine serum and β-mercaptoethanol per manufacturer's instructions. Macrophage differentiation occurred through supplementation with 20 ng/ml Phorbol 12-myristate 13-acetate (Sigma Aldrich, St. Louis, Mo.) for 48 h.

### Bacterial Strains:

Sputum isolates of nontypeable H. influenzae and P. aeruginosa obtained from patients with COPD at the Department of Veterans Affairs, Western New York Healthcare System were used (S. Sethi, N. Evans, B. J. Grant, T. F. Murphy, N Engl J Med 347, 465 (Aug. 15, 2002); C. S. Berenson, M. A. Garlipp, L. J. Grove, J. Maloney, S. Sethi, J Infect Dis 194, 1375 (Nov. 15, 2006); T. F. Murphy et al., Am J Respir Crit Care Med 177, 853 (Apr. 15, 2008); T. F. Murphy, A. L. Brauer, A. T. Schiffmacher, S. Sethi, Am J Respir Crit Care Med 170, 266 (Aug. 1, 2004)). For bacterial growth, H. influenzae was cultured on chocolate agar or grown in brain heart infusion broth supplemented with 20 μg/mlNAD and 10 μg/ml hemin at 35° C. in 5% CO2. P. aeruginosa was cultured on LB agar plates or in LB broth at 37° C. For FITC-labeling of bacteria, heat inactivated PA and NTHI (10⁹ CFU/ml) were resuspended in 1 ml of labeling buffer (0.1 M NaHCO3, pH 9.2) and incubated with fluorescein isothiocyanate (FITC) (Sigma) under constant stiffing in the dark at room temperature for 1 h. Finally, bacteria were washed three times with PBS and dialyzed overnight against PBS. FITC-labeled bacteria were resuspended with PBS at a concentration of 10⁹/ml.

### Clearance of Bacteria In Vitro:

After treatment, macrophages (10⁵ cells) were incubated with bacteria (PA or NTHI) for 4 h. Subsequently, 100 μL of cell free culture medium was aseptically plated and cultured on tryptose blood and or chocolate agar plates at 37° C. The number of bacterial colonies was counted after 24 h.

### Phagocytosis Assay:

Bacterial phagocytosis was quantified using a flow cytometry analysis. Macrophages were incubated with FITC-labeled bacteria (PA or NTHI) at a bacteria/alveolar macrophage ratio of 50:1 for 4 h at 37° C. with continuous gentle rotation. At the end of the incubation, cells were washed three times with cold PBS and resuspended in PBS containing 0.2% (wt/vol) trypan blue to quench fluorescence caused by the binding of bacteria to the surface of the cells and 1% (vol/vol) paraformaldehyde to fix the cells. Flow cytometry was conducted using a fluorescence activated cell sorter (FACSCalibur, BD Biosciences, San Diego, Calif.) and associated software (CellQuest, BD Biosciences). Phagocytic activity was expressed as the mean fluorescence intensity (MFI) obtained using applicable software (FlowJo, Tree Star, Inc., Ashland, Oreg.). For bacterial binding assay, macrophages were treated with cytochalasin D prior to incubation with FITC-PA and analyzed by FACS for the acquisition of fluorescence as an indication of macrophage association with bacteria.

### Intracellular Killing:

To determine intracellular killing, macrophages were treated with vehicle (DMSO) or sulforaphane (10 μM) for 24 hours. Cells were then washed and the medium was replaced with serum-free medium. After the medium replacement, approximately 10⁵ CFUs of PA were added to each well. After 1 h, the medium was removed and cells were washed, and the gentamicin-containing medium was added to kill bound, unphagocytosed bacteria. Cells were lysed at the indicated time points, and lysates were plated to determine viable intracellular bacteria.

Animals and Treatments:

C57BL/6J mice were housed under controlled conditions for temperature and humidity, using a 12-hour light/dark cycle. At 8 weeks of age, mice were exposed to CS for (2.5 h/day for 5 days/week) for 1 week or 6 months using a machine that produces cigarette smoke (TE-10, Teague Enterprises) and reference cigarettes (2R4F) with a total suspended particle concentration of 250 mg/m³ as previously described (T. E. Sussan et al., Proc Natl Acad Sci USA, (Dec. 22, 2008)). Following exposure to CS, mice were inoculated with PA or NTHI at a dose of 10³ CFU in 50 μl of PBS intranasally. For sulforaphane administration (0.5 mg/mouse of R, S-sulforaphane in PBS), a nebulizer (Aerogen, Inc., Galway, Ireland) designed specifically for small animals was used.

### Bronchoalveolar Lavage in Mice:

BAL fluid analysis and cell counts with differential staining were carried out as described previously (T. Rangasamy et al., J Clin Invest 114, 1248 (November, 2004)). For analyzing bacterial colonization in lungs, 100 μl of BAL fluid were diluted serially in sterile 0.9% NaCl and aseptically plated and cultured on appropriate (blood [PA] or chocolate [NTHI]) agar plates. After 24 h, the number of CFU was counted.

### Antibodies and Flow Cytometry:

Antibodies against MARCO were purchased from Santa Cruz Biotechnology (mouse) and Hycult (human), and Nrf2 antibody was purchased from Santa Cruz (mouse and human). Flow cytometry analysis was performed using a Beckton Dickinson FACS-Calibur Flow cytometer (BD, Franklin Lakes, N.J.). Analysis of flow cytometry data was performed using applicable software (FLOWJO, Tree Star, Inc., Ashland, Oreg.).

siRNA Transfection in Human Alveolar Macrophages: COPD alveolar macrophages plated at 80% confluence in a 96-well plate were transfected with Nrf2 siRNA (Dharmacon) and or control ssRNA using a transfection reagent optimized for macrophage transfections (JetPEI, Polyplus Transfection-SA, Illkirch, France) according to manufacturer's protocol. Knockdown of the Nrf2 gene and Nrf2 target genes were quantified by real-time reverse transcriptase-polymerase chain reaction (RT-PCR) 24 h after transfection.

### Microarray and Quantitative Real-Time RT-PCR:

Total RNA was extracted and purified from macrophages through cell lysis and binding to a silica membrane (RNeasy Mini Kit Qiagen Inc., Valencia, Calif.) according to the manufacturer's recommended protocol. Quality assessment was determined using a microfluidic chip (RNA Nano LabChip analyzed on an Agilent Bioanalyzer 2100). Processing of templates for GeneChip analysis and data analysis was in accordance with methods described previously (D. J. Blake et al., Am J Respir Cell Mol Biol, (Jun. 11, 2009)). Quantitative real-time RT-PCR analyses were conducted using assay on demand probe sets (Applied Biosystems), and reactions were analyzed using a real time fluorescent PCR system (ABI 7000 Taqman system, Applied Biosystems). GAPDH was used for normalization.

### Identification of AREs in the Promoter of MARCO:

To identify the presence and location of AREs in the MARCO (Accession No. 17167) promoter, the 2-kb upstream region from the translation start site was downloaded from the NCBI database. AREs in this region were identified as previously described (C. J. Harvey et al., Free Radic Biol Med, (Nov. 5, 2008); A. Singh et al., Free Radic Biol Med, (Nov. 1, 2008)).

### Plasmids and Mutagenesis:

The 5′ flanking region of the mouse MARCO promoter region was PCR amplified from genomic DNA isolated from murine blood, cloned into pCR2.1 (Invitrogen), and subsequently cloned into the pGL3 Basic vector (Promega, Madison, Wis., USA) (C. J. Harvey et al., Free Radic Biol Med, (Nov. 5, 2008); A. Singh et al., Free Radic Biol Med, (Nov. 1, 2008)). Two deletion constructs (−1708 to +76 and −1005 to +76) were generated. The primers used for amplification were as follows: AAAACCACTGAGGCA (ARE1-2 forward), ATGGAACCCAGAG (ARE1 forward), and GATTTCCATGTGGGTGGAAC (reverse primer for all constructs).

Individual AREs identified in the mouse MARCO promoter region were PCR amplified from the ARE1-2 constructs and ligated into pCR2.1 (Invitrogen). A Kpn1-Xho1 fragment from this construct was cloned into the pTAL luciferase reporter vector (BD Biosciences, San Jose, Calif., USA). The forward primers used for amplification were as follows: ARE1 TCCCCCACTTCTGATGATGT, ARE 2 AAAACCACTGAGGCATCGAC. The reverse primers used for amplification were as follows: ARE1 GTTCCACCCACATGGAAATC, ARE2 CACAAACCTCTGGGTTCCAT. Mutated (mu) ARE sequences were generated by using a site-directed mutagenesis kit from Stratagene (La Jolla, Calif., USA) according to manufacturer's instructions. Primers containing the mu-ARE sequences (mu-ARE2, CTTAATGCACAAACCAAAAGGCATTCAG and mu-ARE1, ATATGTATCCTGCCACCTGGCACCAT) were used. The mutation in each promoter was verified by sequencing. The NQO1 ARE was used as a positive control (R. Tirumalai, T. Rajesh Kumar, K. H. Mai, S. Biswal, Toxicol Lett 132, 27 (Jun. 7, 2002)).

DNA Transfection and Luciferase Activity: Cells were transfected at 80% confluence using a cationic lipid (Lipofectamine 2000, Invitrogen), and luciferase activity was assessed as previously described (C. J. Harvey et al., Free Radic Biol Med, (Nov. 5, 2008); A. Singh et al., Free Radic Biol Med, (Nov. 1, 2008)). A ratio of luciferase activity to Renilla luciferase activity was determined to normalize for transfection efficiency.

### Chromatin Immunoprecipitation Assay:

BMDM macrophages were isolated and derived from Keap1 LysM and Keap1^{fl/fl} mice, and the chromatin immunoprecipitation (CHIP) assay was performed using a commercially available kit (Upstate Biotechnology, Lake Placid, N.Y., USA) as previously described (C. J. Harvey et al., Free Radic Biol Med, (Nov. 5, 2008)). The MARCO primer sequences were as follows: (1) ARE1 forward, TGCTATTAACAAAGATCTCT; reverse, CCAGGCACCCATATCTCAGT; (2) ARE2 forward, TCCTCACAGATATGGAGCTC; reverse, TCTGCTTGCTGCCTAGAGGT.

ChIP assay was performed using either anti-Nrf2 or anti-RNA Pol2 antibodies (Santa Cruz Biotechnology, Santa Cruz, Calif.).

MARCO shRNA:

THP-1 cells were infected with lentivirus particles containing MARCO shRNA (Open Biosystems, Huntsville, Ala.). Puromycin supplemented medium ensured positive selection of lentivirus plasmid-expres sing cells, and knockdown of MARCO mRNA and protein was confirmed by both real-time PCR and western blot.

### Statistical Analysis

Results are presented as the means±SE. Statistical comparisons were performed by paired Student tests. A value of p<0.05 was considered statistically significant.

The following methods and materials were used in Examples 9 through 15.

### Patient Characteristics

This study was approved by the institutional review board of Johns Hopkins University. Patients with COPD were recruited from clinical populations at Johns Hopkins Hospital and Johns Hopkins Bayview Medical Center. Frozen peripheral lung tissue samples were obtained from the NHLBI Lung Tissue Research Consortium. Clinical information and patient characteristics are summarized in FIGS. 22 and 23.

### Mouse Studies

Mice (C57BL/6 strain, 8-10 weeks old) were used for the studies. Exposure to CS (1 month) was carried out as described previously (Malhotra, D., et al., Am J Respir Crit Care Med 178, 592-604 (2008)). Residential peritoneal macrophages (PMs) isolation was carried out as described previously (Singh, A., et al., PLoS Med 3, e420 (2006)). Alveolar macrophages from mouse were isolated from bronchoalveolar lavage (BAL) fluid (Crimi, E., et al., Free Radic Biol Med 40, 398-406 (2006)).

### Treatment

Macrophages (human and mouse) were adherence purified and were >95% as determined by the morphologic appearance of histologically stained preparations (Diff-Quick). Percent viability of macrophages by trypan blue exclusion was >90%. For inducing corticosteroid resistance, PMs were exposed to cigarette smoke condensate (CSC, 100 μg/ml; Murty Pharmaceuticals, Lexington, Ky.) for 16 h. Exposure to S-nitrosoglutathione (GSNO, 10 μM) was limited to 4 h. Cultured macrophages were treated with sulforaphane (5 μM for 16 h; LKT Laboratories, Inc., St. Paul, Minn.), glutathione-reduced ethyl ester (GSH-e, 1 mM for 4 h, Sigma Aldrich, St. Louis, Mo.), L-nitroarginine methyl ester (L-NAME) (1 mM for 4 h; Sigma) and or respective vehicle (DMSO or PBS) in RPMI 1640 culture medium with 10% FBS and 1% penicillin-streptomycin (Invitrogen, Carlsbad, Calif.) for 16 h. For assessing corticosteroid responsiveness, macrophages were incubated with dexamethasone (1 μM, Sigma) for 1 h followed by LPS (100 ng/ml, Sigma) for additional 4 h. Culture medium (serum free) was used for analyzing cytokines (human IL-8 and mouse IL-6 or MCP-1) using ELISA kits (R&D systems, Minneapolis, Minn.) and cell lysate was used for gene expression, enzymatic activity, GSH, NO, or immunoblot analysis.

### HDAC Enzymatic Activity, GSH, and NO Analysis:

HDAC activity was measured using a fluorescent derivative of ε-acetyl-lysine from Enzo life sciences (Farmingdale, N.Y.) according to the manufacturer's recommendations. HDAC2 was immunoprecipitated for analysis of enzymatic activity. GSH and NO levels were determined by using monochlorobimane and DAF-FM diacetate dye (Invitrogen, Carlsbad, Calif.), respectively.

### S-Nitrosylation and Other Assays:

S-nitrosylation of proteins was analyzed by anti-SNO antibody, biotin switch assay, and or 2,3-diaminonapthalene (DAN) assay. DAN assay was performed using the nitrate/nitrite fluorometric assay kit from Cayman chemicals (Ann Arbor, Mich.). Immuno-precipitation, immunoblot, ChIP, and gene expression analysis were carried out as described previously²³′⁵⁶. Detail methodology is described in supplementary data.

### Cytokine Assays

For human IL-8 and mouse IL-6 and MCP-1 proteins, Quantikine ELISA kits from R&D systems (Minneapolis, Minn.) were used according to the manufacturer's instructions.

### Immunoprecipitation of HDAC2

Macrophage cell or lung tissue extracts were prepared by using 100 μL of immunoprecipitation buffer (50 mM Tris-HCl, pH 8.0, 150 mM NaCl, 1.0% Triton X-100, 0.5% NP-40, 0.1% SDS, 0.5% deoxycholate, and complete protease inhibitor cocktail) from Roche (Morristown, N.J.). The lysis mixture was incubated on ice for 15 minutes and microcentrifuged for 10 minutes at 4° C. Extracts were precleared with 20 μL protein A/G sepharose beads (a 50:50 mix) from Thermo scientific (Rockford, Ill.) and 2 μg of normal IgG. After microcentrifugation, 20 μL of protein A/G sepharose conjugated with 5 μg of antibody was used to precipitate HDAC2 (Santa Cruz Biotechnology, Santa Cruz, Calif.) overnight at 4° C. with rotation. The immune complexes were pelleted with gentle centrifugation, washed twice with 1 mL of immunoprecipitation buffer, and divided equally for the final wash for the activity assay and immunoblotting. For the HDAC assay, immunoprecipitates were resuspended in the activity assay buffer (50 mM TrisHCl, pH 8.0; 10% glycerol; 1 mM dithiothreitol; 0.1 mM EDTA, complete protease inhibitor cocktail). For HDAC2-specific western blotting, immune complexes were washed three times with the immunoprecipitation buffer and finally resuspended in Laemmli buffer for the SDS-PAGE.

### DAN Assay

S-nitrosylated proteins were detected by using the nitrate/nitrite fluorometric assay kit from Cayman chemicals (Ann Arbor, Mich.) according to the manufacturer's instructions. For HDAC2-specific nitrosylation detection, cells were lysed in buffer (50 mM Tris-HCl, pH 8.0, 150 mM NaCl, 1% Nonidet P-40, 0.5% deoxycholic acid, 0.1% SDS). These lysates were then subjected to immunoprecipitation by using an anti-HDAC2 antibody as described above. The immunoprecipitates were washed twice with lysis buffer and twice with PBS. The pellets were resuspended in 500 μL of PBS. After the addition of 100 μM HgCl2 and 100 μM DAN, the samples were incubated in the dark at 25° C. for 30 minutes, and 1 M NaOH was added. The generated fluorescent triazole from the reaction of the DAN with the NO released from the HDAC2 protein was measured by using an excitation wavelength of 375 nm and an emission wavelength of 450 nm. As a negative control, the antibody alone in lysis buffer was immunoprecipitated. The resulting background fluorescence intensity was subtracted for each experiment.

### Biotin-Switch Assay

This assay was performed in the dark. Cells were lysed in HEN buffer (250 mM HEPES, 1 mM EDTA, and 100 mM neocuproine) and adjusted to contain 0.4% CHAPS. Samples were homogenized, and free cysteines were blocked for 1 hour at 50° C. in three volumes of blocking buffer (HEN buffer plus 2.5% SDS, HENS) containing methyl methanethiosulfonate (200 mM).

Proteins were acetone precipitated at −20° C. and resuspended in 100 μL HENS solution. After adding fresh ascorbic acid (20 mM) and 1 mM biotin-HPDP (Thermo scientific, Rockford, Ill.), proteins were incubated at room temperature for 1 hour. Biotinylated proteins were resuspended in 100 μL HENS buffer containing 200 μL of neutralization buffer (20 mM HEPES, 100 mM NaCl, 1 mM EDTA, 0.5% Triton X-100) and precipitated with 50 μL of streptavidin—agarose beads at room temperature for 1 hour. The beads were washed five times at 4° C. by using neutralization buffer containing 600 nM NaCl. Biotinylated proteins were eluted by using 50 μL elution buffer (20 mM HEPES, 100 mM NaCl, 1 mM EDTA, 100 mM β-mercaptoethanol) and heated at 95° C. for 5 minutes in reducing SDS-PAGE loading buffer followed by immunoblotting with specific antibodies.

### Nuclear Extract Preparation

Nuclear extracts were prepared by cellular lysis and differential centrifugation (NE-PER kit from Promega, Madison, Wis.) according to the manufacturer's instructions for preparing the nuclear extracts.

### Immunoblot Assays

All immunoblots were performed by using protocols as described previously (Barnes, P. J., N Engl J Med 343, 269-280 (2000)). Rabbit anti-HDAC2 antibody from Santa Cruz Biotechnology (Santa Cruz, Calif.) was used at 1:500 dilutions. Rabbit anti-5-nitroso-cysteine antibody from Alpha diagnostics was used at 1:500 dilutions. Rabbit anti-nitro-tyrosine antibody from Cell Signaling Technology, was used at 1:250 dilution. Rabbit anti-ubiquitin antibody from Santa Cruz Biotechnology (Santa Cruz, Calif.) was used at 1:1000 dilutions. Rabbit anti-GR antibody from Santa Cruz Biotechnology (Santa Cruz, Calif.) was used at a dilution of 1:1000. Rabbit anti-MMP9 antibody from Sigma Aldrich was used at 1:500 dilutions. Rabbit anti-acetyl-lysine antibody from Abcam was used at 1:750 dilutions. Rabbit anti-lamin-B1 from Santa Cruz Biotechnology (Santa Cruz, Calif.) was used at 1:1000 dilutions as a loading control. The Image J program was used for densitometry analyses.

Quantitative Real-Time PCR (qRT-PCR) Assays

Total RNA was extracted from the cells by lysis and specific binding to a silica membrance (Qiagen RNeasy kit, Qiagen, Valencia, Calif.), and qRT-PCR was performed by using primers and probe sets for human IL-8, NQO1, GCLM, and iNOS (Assay-on-Demand, Applied Biosystems, Foster City, Calif.). A real-time fluorescent PCR system (ABI 7000 Taqman system, Applied Biosystems) was used to perform these assays. β-actin was used as the normalization control.

### Chromatin Immunoprecipitation (ChIP)—PCR Assays

Chromatin immunoprecipitation was performed as described previously (Hogg, J. C. & Timens, W., Annu Rev Pathol (2008)). Briefly, cells were fixed in ˜1% formaldehyde in cell growth media for 10 minutes followed by glycine fixation. Cells were then sonicated in ChIP-lysis buffer containing a protease inhibitor cocktail after incubation on ice for 30 minutes. During sonication, the samples were spun at maximum speed for 12 minutes in a cold microcentrifuge. The supernatants were then addressed as input samples. A part of the input sample was precleared by using a 50% slurry of protein A/G beads prepared in immunoprecipitation (IP) buffer for 2 hours in the cold, following which the beads were centrifuged out and the supernatant was used for IP. Then the precleared samples were incubated with 2 μg of panacetylated histone H4 antibody (ChIP assay kit; Upstate Biotechnology) or anti-HDAC2 antibody from Santa Cruz Biotechnology. As a control, anti-rabbit IgG antibody was added for 1 hour in the cold. The protein A/G bead slurry was added to each reaction and was rotated overnight. The next day, the beads were washed extensively, and the DNA was reverse crosslinked, eluted in elution buffer, and stored at −80° C. until used for sequencing or PCR validation assays. The eluted DNA was checked for 100-300 bp fragment enrichments by using 8% polyacrylamide gel electrophoresis (data not shown). This immunoprecipitated DNA was used for PCR amplification by using the specific gene (IL-8 for humans and MCP-1 for mice) promoter PCR assays. PCR primers were used for the IL-8 promoter (forward: 5′-TTCCTTCCGGTGGTTTCTTC-3′ and reverse: 5′-GGGCCATCAGTTGCAAATC-3′) and the MCP-1 promoter (forward: 5′-CAAGCCAGAGCTCAGACTAGGCCT-3′ and reverse: 5′-CACAGGAGGCAGCGCAAATGTGA-3′).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of restoring corticosteroid responsiveness in a subject in need thereof, the method comprising contacting a cell of the subject with an agent that increases Nrf2 biological activity in the cell, thereby restoring corticosteroid responsiveness in the subject.

2. The method of claim 1, wherein the subject has chronic obstructive pulmonary disease, asthma, severe asthma, acute graft-versus host disease, autoimmune inner ear disease, inflammatory bowel disease, or rheumatoid arthritis.

3. A method of restoring corticosteroid responsiveness in a subject with chronic obstructive pulmonary disease (COPD) comprising administering an effective amount of an agent that stimulates Nrf2 activity to the subject, thereby restoring corticosteroid responsiveness in the subject.

4. The method of claim 1, wherein the method reverses corticosteroid resistance.

5. The method of claim 1, wherein the agent is sulforaphane or another agent listed in Table 1.

6. The method of claim 1, wherein the method further comprises administering a corticosteroid.

7. The method of claim 6, wherein the corticosteroid is selected from the group consisting of dexamethasone, flunisolide, fluticasone propionate, triamcinolone acetonide, beclomethasone dipropionate, budesonide, prednisone, prednisolone, and methylprednisolone.

8. A method of treating or preventing a respiratory infection in a subject, the method comprising contacting a cell of the subject with an agent that increases Nrf2 biological activity in the cell, thereby treating or preventing the respiratory infection.

9. The method of claim 8, wherein the subject has an acute respiratory infection, chronic bronchitis, cystic fibrosis or an immunodeficiency syndrome that reduces or otherwise compromises the efficacy of the subject's immune system.

10. The method of claim 8, wherein the subject is a smoker, has emphysema, or has COPD.

11. The method of claim 8, wherein the infection is associated with Pseudomonas aeruginosa, nontypeable Haemophilus influenzae, Moraxella catarrhalis, streptococcus pneumonia, staphylococcus aureus, Rhinovirus, coronovirus, influenza A and B, parainfluenza, Adenovirus, or Respiratory syncytial virus.

12. The method of claim 8, wherein the cell is an alveolar macrophage.

13. A method for increasing macrophage bactericidal activity, the method comprising contacting a macrophage with an agent that increases Nrf2 activity, thereby increasing macrophage bactericidal activity.

14. A method of increasing bacterial phagocytosis by a macrophage comprising contacting the macrophage with an agent that increases Nrf2 activity, thereby increasing bacterial phagocytosis by the macrophage.

15. The method of claim 10, wherein the macrophage is an alveolar macrophage.

16. A method of treating or preventing bacterial colonization in a tissue or organ of a subject, the method comprising contacting the tissue or organ of the subject with an agent that increases Nrf2 biological activity, thereby treating or preventing bacterial colonization and inflammation.

17. The method of claim 1, wherein the agent is sulforaphane or another agent listed in Table 1.

18. The method of claim 8, wherein the method further comprises administering an antibiotic.

19. The method of claim 1, wherein the method increases Nrf2 transcription or translation.

20. The method of claim 19, wherein the tissue is a mucous membrane and the organ is the lung.

21. The method of claim 1, wherein the agent increases a Nrf2 biological activity selected from the group consisting of binding to an antioxidant-response element (ARE), nuclear accumulation, or the transcriptional induction of target genes.

22. The method of claim 17, wherein the target gene is Marco.

23-29. (canceled)

30. A method for treating an infection in a subject having or at risk of developing chronic obstructive pulmonary disease (COPD), the method comprising administering an effective amount of a Keap1 inhibitor to a subject in need thereof, thereby treating chronic obstructive pulmonary disease.

31-37. (canceled)

38. A method for reversing corticosteroid resistance in a subject having COPD, the method comprising administering an effective amount of an agent that increases Nrf2 biological activity and a corticosteroid to a subject in need thereof, thereby treating or preventing corticosteroid resistance in the subject.

39. (canceled)

40. A method for treating or preventing a pulmonary infection in a subject having or at risk of developing COPD, the method comprising administering an effective amount of an agent that increases Nrf2 biological activity and a corticosteroid to a subject in need thereof, thereby treating or preventing a pulmonary infection in the subject.

41. (canceled)

42. A pharmaceutical composition for the treatment or prevention of a pulmonary inflammatory condition comprising a therapeutically effective amount of an agent that increases a Nrf2 biological activity or Nrf2 expression and an effective amount of a corticosteroid in a pharmaceutically acceptable excipient or
A pharmaceutical composition for the treatment or prevention of corticosteroid resistance comprising a therapeutically effective amount of an agent that increases a Nrf2 biological activity or Nrf2 expression and an effective amount of a corticosteroid in a pharmaceutically acceptable excipient, or
A pharmaceutical composition for treating or preventing a respiratory infection, the composition comprising an effective amount of an agent that increases a Nrf2 biological activity or Nrf2 expression and an effective amount of an antibiotic.

43-52. (canceled)

53. A device for dispersing an effective amount of an agent that increases a Nrf2 biological activity or Nrf2 expression and an effective amount of a corticosteroid into particles and delivering a dose of said particles to lung tissue of a subject.

54. (canceled)

55. A kit for reversing corticosteroid resistance, the kit comprising an effective amount of an agent that increases a Nrf2 biological activity or Nrf2 expression and an effective amount of a corticosteroid and instructions for the use of said kit in the method of claim 1.

56. A kit for treating a respiratory infection, the kit comprising an effective amount of an agent that increases a Nrf2 biological activity or Nrf2 expression and an effective amount of a corticosteroid in a pharmaceutically acceptable excipient, and instructions for the use of said kit in the method of claim 8.
